(19) United States
(12) Patent Application Publication
(10) Pub. No.: US 2002/0065309 A1
(43) Pub. Date: May 30, 2002

(54) Novel Bicyclic and tricyclic pyrrolidine derivatives as GnRH antagonists

(76) Inventors: Ge Peng, Mountain View, CA (US); Mark A. Gallop, Los Altos, CA (US); Tania Chernov-Rogan, Los Altos, CA (US); Stephen D. Yanofsky, Palo Alto, CA (US); Jeffrey Claude Pelletier, Lafayette Hill, PA (US); Daniel Michael Green, Ambler, PA (US)

Correspondence Address: REED & ASSOCIATES, 800 MENLO AVENUE, SUITE 210, MENLO PARK, CA, 94025, US

(21) Appl. No.: 09/860,810

(22) Filed: May 18, 2001

Related U.S. Application Data
(63) Continuation-in-part of application No. 09/633,025, filed on Aug. 4, 2000.
(60) Provisional application No. 60/147,233, filed on Aug. 4, 1999.

(51) Int. Cl. A61K 31/4188; C07D 487/14

(52) U.S. Cl. 514/387; 548/302.4

(57) ABSTRACT

Bicyclic and tricyclic pyrrolidine derivatives are disclosed that are useful as antagonists of the GnRH receptor. Methods for using the novel compounds to treat GnRH-related disorders are also provided, as are pharmaceutical compositions and novel synthetic methods.

### CROSS-REFERENCE TO RELATED APPLICATIONS

This is a continuation-in-part of U.S. patent application Ser. No. 09/633,025 filed Aug. 4, 2000, the disclosure of which is incorporated by reference herein.

### TECHNICAL FIELD

The present invention relates generally to pharmaceuticals and use thereof, and more particularly relates to novel pharmaceutical agents in the form of bicyclic and tricyclic pyrrolidine derivatives. The invention additionally relates to methods of using the novel compounds as GnRH antagonists, to pharmaceutical compositions containing a compound of the invention as the active agent, and to methods for synthesizing the novel compounds provided herein.

### BACKGROUND

Gonadotropin-releasing hormone (GnRH), also referred to as luteinizing hormone-releasing hormone (LHRH), is a decapeptide that is produced in the hypothalamus and when released therefrom acts on the pituitary gland to stimulate the biosynthesis and secretion of various hormones, including luteinizing hormone (LH) and follicle stimulating hormone (FSH). The LH released from the pituitary gland is primarily responsible for the regulation of gonadal steroid production in both sexes, whereas FSH regulates spermatogenesis in males and follicular development in females. Since GnRH was first discovered in 1971, a number of its analogs have been synthesized in the hopes of exploiting their agonistic or antagonistic activity. In particular, GnRH agonists and antagonists have proven effective in the treatment of certain conditions that require inhibition of LH and/or FSH release. For example, GnRH-based therapies have proven to be effective in the treatment of endometriosis, uterine fibroids, polycystic ovarian disease, precocious puberty and several gonadal steroid-dependent neoplasia, most notably cancers of the prostate, breast and ovary. GnRH agonists and antagonists have also been used in assisted reproduction techniques and have been investigated as potential contraceptive agents in both men and women. They have also shown possible utility in the treatment of pituitary gonadotrophe adenomas, sleep disorders such as sleep apnea, irritable bowel syndrome, premenstrual syndrome, benign prostatic hyperplasia (BPH), hirsutism, lupus, including systemic lupus erythematosis (SLE), and as an adjunct to growth hormone therapy in growth hormone deficient children.

Current GnRH antagonists are for the most part GnRH-like decapeptides. In addition, cyclic hexapeptide derivatives having GnRH receptor antagonizing activity have been prepared (Japanese Patent Publication (Kokai) No. 61-191698, 1986), as have certain bicyclic peptide derivatives (Bienstock et al. (1993) J. Med. Chem. 36:3265-3273) and straight-chain peptides (U.S. Pat. Nos. 5,140,009 and 5,171,835). However, since these compounds are all peptides, many problems remain, the most critical of which is poor oral bioavailability. The peptide analogs of GnRH cannot, for this reason, be administered orally to provide the desired therapeutic effect.

Certain non-peptide GnRH antagonists have been proposed, and some offer the possible advantage of oral administration. For example, PCT Publication No. WO 97/14682 describes the use of certain quinoline derivatives as having GnRH receptor antagonizing activity. In addition, PCT Publication No. WO 97/21435 describes the use of substituted indole compounds as GnRH antagonists; such compounds include, for example, the following structure:

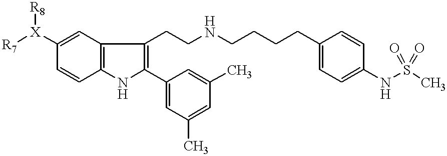

wherein X—R₇R₈ may be, for example, COOCH₂CH₃, CO—N(CH₂CH₂OH), CO—NHCH₂CH₃, or CO—NH-cyclopropyl. Other non-peptide GnRH antagonists are described in European Application No. 0 219 292, in De et al. (1989) J. Med. Chem. 32:2036-2038, and in WO 95/29900, WO 95/28405 and European Application No. 0 679 642.

However, to the best of applicants' knowledge, there are no non-peptide compounds that have sufficient high GnRH receptor antagonizing activity to be used effectively as therapeutic agents. Such compounds would obviously be extraordinarily useful for treating a variety of disorders and diseases, particularly sex-hormone related conditions.

### SUMMARY OF THE INVENTION

The invention is directed to novel compounds useful as antagonists of the GnRH receptor. The compounds are bicyclic or tricyclic pyrrolidine derivatives; the former compounds contain the molecular fragment

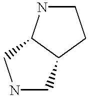

while the latter compounds contain the molecular fragment

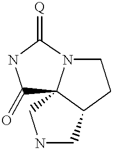

wherein Q is O or S. The tricyclic pyrrolidine derivatives are preferred, and generally comprise compounds having the structural formula

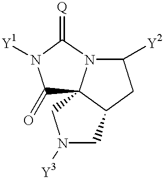

wherein Y¹, Y² and Y³ are independently optionally substituted hydrocarbyl of 1 to 24 carbon atoms as illustrated in Formula (I) below.

The preferred bicyclic and tricyclic pyrrolidine derivatives of the invention have the structural formula (I)

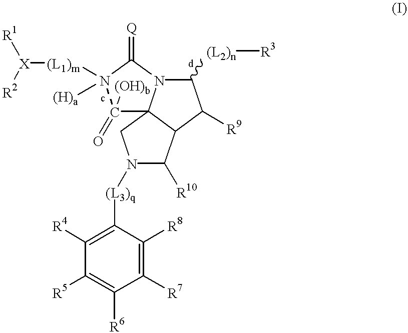

wherein:

L₁, L₂ and L₃ are independently linking groups;

m, n and q are independently 0 or 1;

c is an optional single bond, wherein, when c is present as a single bond, a and b are both 0, while when c is absent, a and b are both 1;

d represents a single bond that is either α or β;

Q is O or S;

X is N or CH;

R¹ and R² are either optionally substituted hydrocarbyl, in which case they may be the same or different, or R¹ and R² are linked together to form a five- or six-membered alicyclic or aromatic ring optionally containing 1 to 3 heteroatoms selected from the group consisting of N, O and S;

R³ is a cyclic structure containing 1 to 3 rings that may be fused or linked, substituted or unsubstituted, wherein 1 or more of the rings may be aromatic and/or heterocyclic;

R⁴, R⁵, R⁶, R⁷ and R⁸ are independently selected from the group consisting of hydrogen, halogen, hydroxyl, alkyl, alkenyl, alkynyl, alkoxy, lower alkyl-substituted alkoxy, amino, lower alkyl-substituted amino, halosubstituted lower alkyl-substituted amino, amido, lower alkyl-substituted amido, halosubstituted lower alkyl-substituted amido, sulfonato, lower alkyl-substituted sulfonato, halosubstituted lower alkyl-substituted sufonato, nitro, nitrile and carboxyl, and, further, when two of R⁴, R⁵, R⁶, R⁷ and R⁸ are ortho to each other, they may together form a five- or six-membered cyclic structure containing 0 to 2 heteroatoms; and

R⁹ and R¹⁰ are independently selected from the group consisting of hydrogen, halogen, hydroxyl, alkyl, alkenyl, alkynyl, alkoxy, amino, lower alkyl-substituted amino, nitro, nitrile and carboxyl,

or are pharmaceutically acceptable salts thereof.

In another embodiment, the invention encompasses pharmaceutical compositions containing a novel compound as provided herein, in combination with a pharmaceutically acceptable carrier. Preferably, such compositions are oral dosage forms and thus contain a carrier suitable for oral drug administration.

In a further embodiment, the invention is directed to a method for antagonizing GnRH in a mammalian individual afflicted with a GnRH-related disorder, comprising administering to the individual a therapeutically effective amount of a bicyclic or tricyclic pyrrolidine derivative as provided herein. That is, since the compounds of the invention are GnRH antagonists, they may be used for treating any of a variety of conditions, diseases and disorders for which GnRH antagonists are useful. Generally, the compounds are used to treat sex hormone related conditions, including sex hormone related cancers, e.g., prostate cancer, uterine cancer, breast cancer, or pituitary gonadotrophe adenomas. Other sex hormone related disorders the present compounds may be used to treat include endometriosis, polycystic ovarian disease, uterine fibroids and precocious puberty. The novel compounds are also useful as contraceptive agents, i.e., for preventing pregnancy in fertile mammalian females. Additionally, the compounds of the invention may be used to treat a variety of other conditions or disorders for which GnRH antagonists are generally recognized to be effective therapeutic agents, including, but not limited to, treatment of sleep apnea, irritable bowel syndrome, benign prostatic hyperplasia and systemic lupus erythematosis.

In a further embodiment of the invention, methods are provided for synthesizing the compounds of the invention, both unsupported and on a solid support. The methods are relatively simple, straightforward, avoid the use of extreme reaction conditions and toxic solvents, and provide the desired products in relatively high yield.

### BRIEF DESCRIPTION OF THE DRAWINGS

FIGS. 1A through 1C schematically illustrate a preferred method for synthesizing a GnRH antagonist of the invention, as described in Example 1.

FIGS. 2A and 2B schematically illustrate a preferred method for synthesizing a GnRH antagonist of the invention, as described in Example 10.

### DETAILED DESCRIPTION OF THE INVENTION

Definitions and Nomenclature:

Before the present compounds, compositions and methods are disclosed and described, it is to be understood that this invention is not limited to specific molecular structures, pharmaceutical compositions, methods of synthesis, or the like, as such may vary. It is also to be understood that the terminology used herein is for the purpose of describing particular embodiments only and is not intended to be limiting.

It must be noted that, as used in the specification and the appended claims, the singular forms “a,” “an” and “the” include plural referents unless the context clearly dictates otherwise. Thus, for example, reference to “a novel compound” in a composition means that more than one of the novel compounds can be present in the composition, reference to “a pharmaceutically acceptable carrier” includes combinations of such carriers, and the like. Similarly, reference to “a substituent” as in a compound substituted with “a substituent” includes the possibility of substitution with more than one substituent, wherein the substituents may be the same or different.

In this specification and in the claims that follow, reference will be made to a number of terms which shall be defined to have the following meanings:

The term “alkyl” as used herein refers to a branched or unbranched saturated hydrocarbon group of 1 to 24 carbon atoms, such as methyl, ethyl, n-propyl, isopropyl, n-butyl, isobutyl, t-butyl, octyl, decyl, tetradecyl, hexadecyl, eicosyl, tetracosyl and the like, as well as cycloalkyl groups such as cyclopentyl, cyclohexyl and the like. The term “lower alkyl” intends an alkyl group of one to six carbon atoms, preferably one to four carbon atoms. The term “cycloalkyl” as used herein refers to a cyclic hydrocarbon of from 3 to 8 carbon atoms, such as cyclopropyl, cyclobutyl, cyclopentyl, cyclohexyl, cycloheptyl and cyclooctyl.

The term “alkenyl” as used herein refers to a branched or unbranched hydrocarbon group of 2 to 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Preferred alkenyl groups herein contain 2 to 12 carbon atoms. The term “lower alkenyl” intends an alkenyl group of two to six carbon atoms, preferably two to four carbon atoms. The term “cycloalkenyl” intends a cyclic alkenyl group of three to eight, preferably five or six, carbon atoms.

The term “alkynyl” as used herein refers to a branched or unbranched hydrocarbon group of 2 to 24 carbon atoms containing at least one triple bond, such as ethynyl, n-propynyl, isopropynyl, n-butynyl, isobutynyl, octynyl, decynyl, and the like. Preferred alkynyl groups herein contain 2 to 12 carbon atoms. The term “lower alkynyl” intends an alkynyl group of two to six carbon atoms, preferably two to four carbon atoms.

The term “alkylene” as used herein refers to a difunctional branched or unbranched saturated hydrocarbon group of 1 to 24 carbon atoms, such as methylene, ethylene, n-propylene, n-butylene, n-hexylene, decylene, tetradecylene, hexadecylene, and the like. The term “lower alkylene” refers to an alkylene group of one to six carbon atoms, preferably one to four carbon atoms.

The term “alkenylene” as used herein refers to a difunctional branched or unbranched hydrocarbon group of 2 to 24 carbon atoms containing at least one double bond, such as ethenylene, n-propenylene, n-butenylene, n-hexenylene, and the like. The term “lower alkenylene” refers to an alkylene group of two to six carbon atoms, preferably two to four carbon atoms.

The term “alkoxy” as used herein intends an alkyl group bound through a single, terminal ether linkage; that is, an “alkoxy” group may be defined as —O-alkyl where alkyl is as defined above. A “lower alkoxy” group intends an alkoxy group containing one to six, more preferably one to four, carbon atoms.

The term “aryl” as used herein, and unless otherwise specified, refers to an aromatic species containing 1 to 3 aromatic rings, either fused or linked, and either unsubstituted or substituted with 1 or more substituents typically selected from the group consisting of lower alkyl, lower alkoxy, halogen, and the like. Preferred aryl substituents contain 1 aromatic ring or 2 fused or linked aromatic rings. The term “arylene” refers to a difunctional aromatic species containing 1 to 3 aromatic rings substituted with 1 or more substituents as above. Preferred arylene substituents contain 1 aromatic ring (e.g., phenylene) or 2 fused or linked aromatic rings (e.g., biphenylylene). The term “aryloxy” as used herein intends an aryl group bound through a single ether linkage; that is, an “aryloxy” group may be defined as —O-aryl where aryl is as defined above.

The term “heterocyclic” refers to a five- or six-membered monocyclic structure or to an eight- to eleven-membered bicyclic heterocycle. The “heterocyclic” substituents herein may or may not be aromatic, i.e., they may be either heteroaryl or heterocycloalkyl. Each heterocycle consists of carbon atoms and from one to three, typically one or two, heteroatoms selected from the group consisting of nitrogen, oxygen and sulfur, typically nitrogen and/or oxygen.

The term “halo” or “halogen” is used in its conventional sense to refer to a chloro, bromo, fluoro or iodo substituent. The terms “haloalkyl,” “haloalkenyl,” “haloamido,” or “haloalkynyl” (or “halogenated alkyl,” “halogenated alkenyl,” “halogenated amido,” or “halogenated alkynyl”) refers to an alkyl, alkenyl or alkynyl group, respectively, in which at least one of the hydrogen atoms in the group has been replaced with a halogen atom.

The term “hydrocarbyl” is used in its conventional sense to refer to a hydrocarbon group containing carbon and hydrogen, and may be aliphatic, alicyclic or aromatic, or may contain a combination of aliphatic, alicyclic and/or aromatic moieties. Aliphatic and alicyclic hydrocarbyl may be saturated or they may contain one or more unsaturated bonds, typically double bonds. The hydrocarbyl substituents herein generally contain 1 to 24 carbon atoms, more typically 1 to 12 carbon atoms, and may be substituted with various substituents and functional groups, or may be modified so as to contain ether and/or thioether linkages. The term “hydrocarbylene” refers to a difunctional hydrocarbyl group, i.e., a hydrocarbyl group that is bound to two distinct molecular moieties.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present. Similarly, the phrase an “optionally present” bond as indicated by a dotted line—in the chemical formulae herein means that a bond may or may not be present.

The term “pyrrolidinyl” refers to a saturated five-membered heterocyclic ring compound containing one ring nitrogen atom and optionally, but not preferably, containing vinyl unsaturation between carbons 3 and 4 of the ring. A “bicyclic pyrrolidinyl” compound as provided herein is a bicyclic compound in which the two cyclic moieties may be fused or linked, and in which one or both of the cyclic moieties are pyrrolidinyl. Similarly, a “tricyclic pyrrolidinyl” compound as provided herein is a tricyclic compound in which the cyclic moieties therein are either fused or linked, and in which one, two or three of the cyclic moieties are pyrrolidinyl.

By the terms “effective amount” or “therapeutically effective amount” of an agent as provided herein are meant a nontoxic but sufficient amount of the agent to provide the desired therapeutic effect. As will be pointed out below, the exact amount required will vary from subject to subject, depending on the species, age, and general condition of the subject, the severity of the condition being treated, and the particular GnRH antagonist and mode of administration, and the like. Thus, it is not possible to specify an exact “effective amount.” However, an appropriate “effective” amount in any individual case may be determined by one of ordinary skill in the art using only routine experimentation.

By “pharmaceutically acceptable carrier” is meant a material which is not biologically or otherwise undesirable, i.e., the material may be administered to an individual along with the selected active agent without causing any undesirable biological effects or interacting in a deleterious manner with any of the other components of the pharmaceutical composition in which it is contained. Similarly, a “pharmaceutically acceptable” salt of a novel compound as provided herein is a salt or ester which is not biologically or otherwise undesirable.

The terms “treating” and “treatment” as used herein refer to reduction in severity and/or frequency of symptoms, elimination of symptoms and/or underlying cause, prevention of the occurrence of symptoms and/or their underlying cause, and improvement or remediaton of damage. Thus, for example, the present method of “treating” a disorder that is responsive to a GnRH antagonist, as the term “treating” is used herein, encompasses both prevention of the disorder in a predisposed individual and treatment of the disorder in a clinically symptomatic individual. Thus, “treatment” of breast cancer as the term is used herein is intended to refer to both prevention and treatment of the disease.

In the molecular structures herein, the use of bold and dashed lines to denote particular conformation of groups follows the IUPAC convention. The symbols “β” and “β” indicate the specific stereochemical configuration of a substituent at an asymmetric carbon atom in a chemical structure as drawn. Thus “α,” denoted by a broken line, indicates that the group in question is below the general plane of the molecule as drawn, and “β” denoted by a bold line, indicates that the group at the position in question is above the general plane of the molecule as drawn.

The Novel Compounds:

The invention provides novel compounds useful as GnRH antagonists, the compounds having the structure of formula (I)

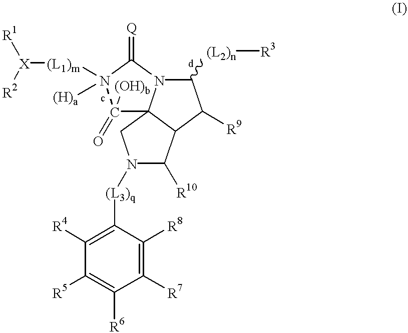

wherein L₁, L₂, L₃, m, n, q, a, b, c, d, Q, X and R¹ through R¹⁰ are as defined above. It will be appreciated by those skilled in the art that structure (I) encompasses two different diastereomers at the central ring structure, as follows:

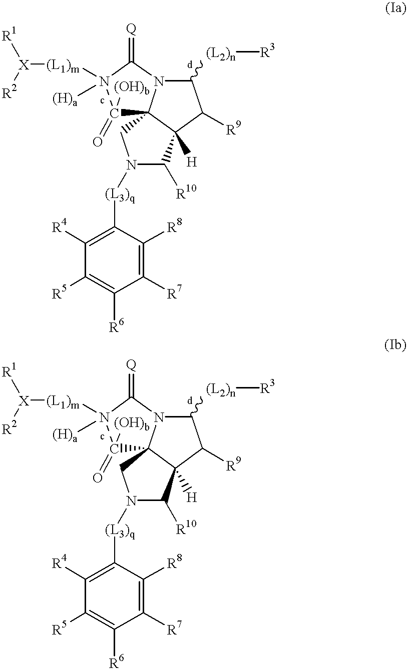

It is applicants' intent to include both diastereomers within the scope of the present invention.

The various substituents are defined as follows:

L₁ is a linking group that may or may not be present, as m may be either 0 or 1. When L₁ is present, i.e., when m is 1, it is generally hydrocarbylene, typically of 1 to 24 carbon atoms, either unsubstituted or substituted with one or more non-hydrogen, non-carbon atoms and one or more functional groups. Preferably, L₁ is alkylene, and most preferably is lower alkylene.

L₂ is also a linking group that may or may not be present, as n may be either 0 or 1. When L₂ is present, i.e., when n is 1, it is generally hydrocarbylene such as alkylene or alkenylene, typically of 1 to 24 carbon atoms, either unsubstituted or substituted with one or more non-hydrogen, non-carbon atoms and one or more functional groups. Preferably, when L₂ is present, it is alkylene, and most preferably is lower alkylene. However, in a preferred embodiment, n is 0 and L₂ is therefore absent.

L₃ is a linking group that may or may not be present, as q may be either 0 or 1. When L₁ is present, i.e., when m is 1, it is generally hydrocarbylene, typically of 1 to 24 carbon atoms, either unsubstituted or substituted with one or more non-hydrogen, non-carbon atoms and one or more functional groups. Preferably, L₁ is alkylene, more preferably lower alkylene, and most preferably methylene.

In tricyclic pyrrolidine derivatives, “c” represents a single bond, and therefore a and b are both 0. In bicyclic pyrrolidine derivatives, “c” is absent, and a and b are both 1.

The bond at “d” may be either α or β, but is preferably β.

Q is O or S; preferably, Q is O.

X is N or CH; preferably, X is N.

R¹ and R² are either optionally substituted hydrocarbyl, in which case they may be the same or different, or R¹ and R² are linked together to form a five- or six-membered alicyclic or aromatic ring optionally containing 1 to 3 heteroatoms selected from the group consisting of N, O and S. Preferably, R¹ and R² are linked to form a ring. When X is N, preferred rings include, but are not limited to, the following: morpholino (i.e., R¹ and R² together form —CH₂—CH₂—O—CH₂—CH₂—); piperazinyl (i.e., R¹ and R² together form —CH₂—CH₂—NH—CH₂—CH₂—); piperazinyl substituted on the ring nitrogen atom with lower alkyl, phenyl, benzyl, or —CO-alkyl (i.e., R¹ and R² together form —CH₂—CH₂—NR—CH₂—CH₂—); piperidinyl (i.e., R¹ and R¹ together form —(CH₂)₅—); pyrrolidinyl (i.e., R¹ and R² together form —(CH₂)₄—); pyridyl (i.e., R¹ and R² together form —CH═CH—CH═CH—CH═); pyrrolyl (i.e., R¹ and R² together form —CH═CH—CH═CH—); and the like. When X is CH, preferred rings, include, but are not limited to, the following: 4-piperidinyl (i.e., R¹ and R² together form —CH₂—CH₂—NH—CH₂—CH₂—); 3-pyrrolidinyl (i.e., RI and R² together form —CH₂—NH—CH₂—CH₂—); 4-pyridyl (i.e., R¹ and R² together form —CH═CH—N═CH—CH═); 2-pyridyl (i.e., R¹ and R² together form ═N—CH═CH—CH═CH—); pyranyl (i.e., R¹ and R² together form —CH═CH—O—CH₂—CH═); and the like. When R¹ and R² represent individual hydrocarbyl substituents, i.e., are not linked to form a ring as just described, they are typically alkyl groups, preferably lower alkyl, either unsubstituted or substituted with alkyl, alkenyl, alkoxy, cyclooxyalkyl, amino, nitro, halogen, hydroxyl or carboxyl groups.

R³ is a cyclic structure containing 1 to 3 rings that may be fused or linked, wherein 1 or more of the rings may be aromatic and/or heterocyclic. Preferred R³ moieties are phenyl and naphthalenyl, substituted with 0 to 2 substituents selected from the group consisting of hydroxyl, lower alkoxy, amino, and di(lower alkyl)amino.

R⁴, R⁵, R⁶, R⁷ and R⁸ are independently selected from the group consisting of hydrogen, halogen, hydroxyl, alkyl, alkenyl, alkynyl, alkoxy, amino, lower alkyl-substituted amino, halosubstituted lower alkyl-substituted amino, amido, lower alkyl-substituted amido, halosubstituted lower alkyl-substituted amido, sulfonato, lower alkyl-substituted sulfonato, halosubstituted lower alkyl-substituted sufonato, nitro, nitrile and carboxyl, and, further, when two of R⁴, R⁵, R⁶, R⁷ and R⁸ are ortho to each other, they may together form a five- or six-membered cyclic structure containing 0 to 2 heteroatoms. In a preferred embodiment, two of R⁴, R⁵, R⁶, R⁷ and R⁸ are hydrogen, and the remainder are independently selected from the group consisting of hydrogen, methoxy, carboxyl, nitro and bromo. In an alternative preferred embodiment, R⁴, R⁵ and R⁸ are hydrogen, and R⁶ and R⁷ are linked together and represent —O—CH₂—CH₂—O—.

R⁹ and R¹⁰ are independently selected from the group consisting of hydrogen, halogen, hydroxyl, alkyl, alkenyl, alkynyl, alkoxy, amino, lower alkyl-substituted amino, nitro, nitrile and carboxyl.

Preferred compounds of the invention are tricyclic pyrrolidine derivatives having the structural formula (II)

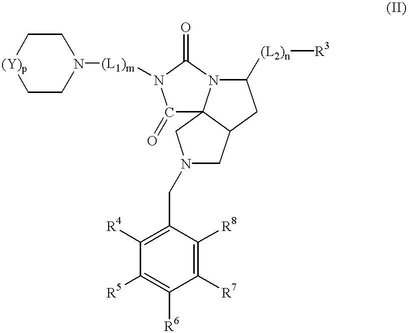

wherein:

L₁ and L₂ are independently lower alkylene linking groups;

m and n are independently 0 or 1;

R³ is phenyl or naphthalenyl, substituted with a single lower alkoxy or di(lower alkyl)amino moiety;

Y is O, S, CH₂ or NR¹¹ wherein R¹¹ is hydrogen, phenyl, benzyl or —(CO)R¹² in which R¹² is lower alkyl, or phenyl, and p is 0 or 1; and

R⁴, R⁵, R⁶, R⁷ and R⁸ are independently selected from the group consisting of hydrogen, halogen, hydroxyl, alkoxy, amido, sulfonado, nitro, and carboxyl, and when two of R⁴, R⁵, R⁶, R⁷ and R⁸ are ortho to each other, they may together form a five- or six-membered cyclic structure containing 0 to 2 heteroatoms, with preferred R⁴, R⁵, R⁶, R⁷ and R⁸ substituents as defined above with respect to formula (I) compounds;

and pharmaceutically acceptable salts thereof.

Specific and preferred compounds of the invention are as follows:

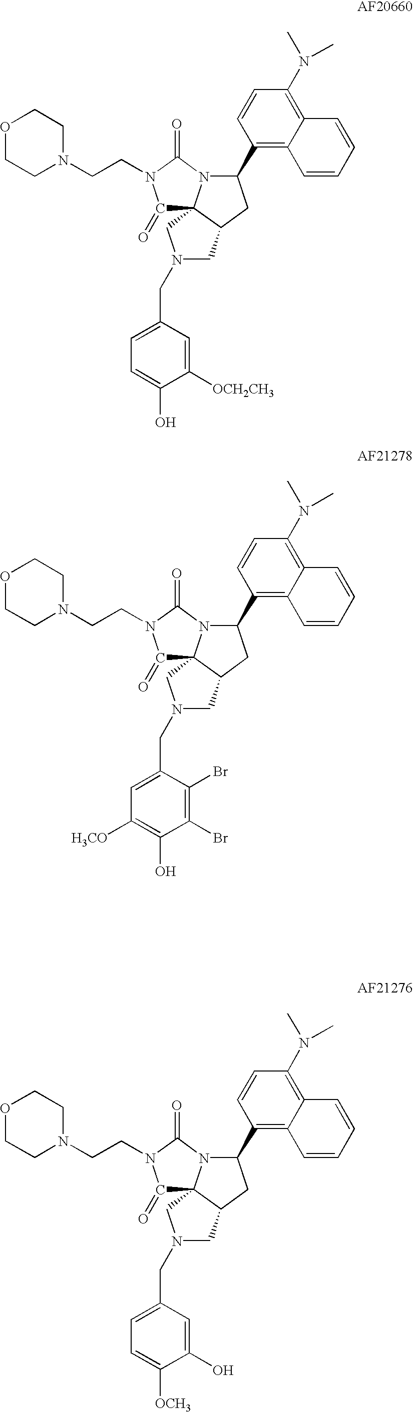

The compounds of the invention may, as noted earlier herein, be in the form of a pharmaceutically acceptable salt. Alternatively, the compounds may be functionalized as esters, amides, or other derivatives, or they may be modified by appending one or more appropriate functionalities to enhance selected biological properties. Such modifications are known in the art and include those which increase biological penetration into a given biological system, increase oral bioavailability, increase solubility to allow administration by injection, and the like.

Salts of the compounds can be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992). Acid addition salts are prepared from the free base (e.g., compounds having a neutral amine group) using conventional means, involving reaction with a suitable acid. Typically, the base form of the compound is dissolved in a polar organic solvent such as methanol or ethanol and the acid is added at a temperature of about 0° C. to about 100° C., preferably at ambient temperature. The resulting salt either precipitates or may be brought out of solution by addition of a less polar solvent. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Preferred acid addition salts of the present compounds are the citrate, fumarate, succinate, benzoate and malonate salts.

Preparation of basic salts of acid moieties which may be present (e.g., carboxylic acid groups) are prepared in a similar manner using a pharmaceutically acceptable base such as sodium hydroxide, potassium hydroxide, ammonium hydroxide, calcium hydroxide, magnesium hydroxide, trimethylamine, or the like.

The novel compounds are chiral in nature and can thus be present in the pharmaceutical compositions herein either in isomerically pure form or in a racemic mixture. In some cases, i.e., with regard to certain specific compounds illustrated herein, chirality (i.e., relative stereochemistry) is indicated. In other cases, it is not, and, as alluded to earlier herein, the invention is intended to encompass both the isomerically pure forms of the compounds shown and the racemic or diastereomeric mixtures thereof. For example, compounds of formula (I) are shown as having a bond

[symbol]

linking the moiety —(L₂)ₙ—R³ to the central ring system. It is intended that the moiety —(L₂)ₙ—R³ may be either α or β, or that a combination of such compounds may be present.

Pharmaceutical Compositions and Modes of Administration

The GnRH antagonists of the invention may be conveniently formulated into pharmaceutical compositions composed of one or more of the compounds in association with a pharmaceutically acceptable carrier. See Remington: The Science and Practice of Pharmacy, 19th Ed. (Easton, Pa.: Mack Publishing Co., 1995), which discloses typical carriers and conventional methods of preparing pharmaceutical compositions that may be used as described or modified to prepare pharmaceutical formulations containing the compounds of the invention.

The compounds may be administered orally, parenterally, transdermally, rectally, nasally, buccally, vaginally or via an implanted reservoir in dosage formulations containing conventional non-toxic pharmaceutically acceptable carriers, adjuvants and vehicles. The term “parenteral” as used herein is intended to include subcutaneous, intravenous, and intramuscular injection. The amount of active compound administered will, of course, be dependent on the subject being treated, the subj ect's weight, the manner of administration and the judgment of the prescribing physician. Generally, however, dosage will be in the range of approximately 0.001 mg/kg/day to 100 mg/kg/day, more preferably in the range of about 0.1 mg/kg/day to 10 mg/kg/day.

Depending on the intended mode of administration, the pharmaceutical compositions may be in the form of solid, semi-solid or liquid dosage forms, such as, for example, tablets, suppositories, pills, capsules, powders, liquids, suspensions, or the like, preferably in unit dosage form suitable for single administration of a precise dosage. The compositions will include, as noted above, an effective amount of the selected active agent in combination with a pharmaceutically acceptable carrier and, in addition, may include other pharmaceutical agents, adjuvants, diluents, buffers, etc.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, etc., an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art; for example, see Remington's Pharmaceutical Sciences, referenced above.

For oral administration, the composition will generally take the form of a tablet or capsule, or may be an aqueous or nonaqueous solution, suspension or syrup. Tablets and capsules are preferred oral administration forms. Tablets and capsules for oral use will generally include one or more commonly used carriers such as lactose and corn starch. Lubricating agents, such as magnesium stearate, are also typically added. When liquid suspensions are used, the active agent is combined with emulsifying and suspending agents. If desired, flavoring, coloring and/or sweetening agents may be added as well. Other optional components for incorporation into an oral formulation herein include, but are not limited to, preservatives, suspending agents, thickening agents, and the like.

Parenteral administration, if used, is generally characterized by injection. Injectable formulations can be prepared in conventional forms, either as liquid solutions or suspensions, solid forms suitable for solution or suspension in liquid prior to injection, or as emulsions. Preferably, sterile injectable suspensions are formulated according to techniques known in the art using suitable dispersing or wetting agents and suspending agents. The sterile injectable formulation may also be a sterile injectable solution or a suspension in a nontoxic parenterally acceptable diluent or solvent. Among the acceptable vehicles and solvents that may be employed are water, Ringer's solution and isotonic sodium chloride solution. In addition, sterile, fixed oils are conventionally employed as a solvent or suspending medium. A more recently revised approach for parenteral administration involves use of a slow release or sustained release system, such that a constant level of dosage is maintained. See, e.g., U.S. Pat. No. 3,710,795.

The compounds of the invention may also be administered through the skin or mucosal tissue using conventional transdermal drug delivery systems, wherein the agent is contained within a laminated structure that serves as a drug delivery device to be affixed to the skin. In such a structure, the drug composition is contained in a layer, or “reservoir,” underlying an upper backing layer. The laminated structure may contain a single reservoir, or it may contain multiple reservoirs. In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. Transdermal drug delivery systems may in addition contain a skin permeation enhancer. That is, because the inherent permeability of the skin to some drugs may be too low to allow therapeutic levels of the drug to pass through a reasonably sized area of unbroken skin, it is necessary to coadminister a skin permeation enhancer with such drugs. Suitable enhancers are well know in the art and include, for example, dimethylsulfoxide (DMSO), dimethyl formamide (DMF), N,N-dimethylacetamide (DMA), decylmethylsulfoxide (C₁₀MSO), C₂-C₆ alkanediols, and the 1-substituted azacycloheptan-2-ones, particularly 1-n-dodecylcyclazacycloheptan-2-one (available under the trademark Azone® from Whitby Research Incorporated, Richmond, Va.), alcohols, and the like.

The pharmaceutical compositions of the invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Preferred formulations for vaginal drug delivery are ointments and creams. Ointments are semisolid preparations that are typically based on petrolatum or other petroleum derivatives. Creams containing the selected active agent are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant.

The specific ointment or cream base to be used, as will be appreciated by those skilled in the art, is one that will provide for optimum drug delivery. As with other carriers or vehicles, an ointment base should be inert, stable, nonirritating and nonsensitizing. Also preferred are vaginal suppositories. Suppositories may be formulated using conventional means, e.g., compaction, compression-molding or the like, and will contain carriers suited to vaginal drug delivery, typically a bioerodible material which provides for the desired drug release profile.

Formulations for buccal administration include tablets, lozenges, gels and the like. Alternatively, buccal administration can be effected using a transmucosal delivery system.

The pharmaceutical compositions of the invention may also include one or more additional active agents, i.e., compounds other than those disclosed and claimed herein. For example, the compositions may also include steroids, e.g.: androgenic agents such as testosterone, testosterone esters, androsterone, androstenediol, dehydroepiandrosterone (DHEA; also termed “prasterone”), 4-dihydrotestosterone (DHT; also termed “stanolone”), and 5α-dihydrotestosterone; estrogens such as: estradiol (i.e., 1,3,5-estratiene-3,17β-diol, or “β-estradiol”) and its esters, 17α-estradiol; ethynylestradiol (i.e., 17α-ethynylestradiol) and esters and ethers thereof, estrone and its esters and derivatives, mestranol, and the like; and progestins such as cyproterone, cyproterone acetate, desogestrel, 3-ketodesogestrel, levonorgestrel, megestrol, norethisterone, progesterone, and the like.

Utility:

The compounds of the invention are useful to treat a mammalian individual afflicted with a GnRH-related disorder; generally, the “GnRH-related disorder” is a sex hormone related condition such as a sex hormone dependent cancer. Sex hormone dependent cancers include, for example, prostate cancer, uterine cancer, breast cancer, or pituitary gonadotrophe adenomas. Other sex hormone related conditions that the present compounds may treat are endometriosis, polycystic ovarian disease, uterine fibroids and precocious puberty. The novel compounds are also useful as contraceptive agents, i.e., in a method for preventing pregnancy in fertile mammalian females. The compounds are additionally useful to treat any condition, disease or disorder for which GnRH antagonists are recognized has having a beneficial effect; for example, the compounds of the invention are useful in treating sleep apnea, irritable bowel syndrome, benign prostatic hyperplasia, and systemic lupus erythematosis. For those compounds of the invention that are orally active, oral administration to treat the aforementioned conditions and disorders is preferred over other routes of administration.

Synthetic Methods:

The novel bicyclic and tricyclic pyrrolidine compounds of the invention may be synthesized on a solid support and cleaved therefrom following completion of synthesis or may be synthesized without the use of a solid support. The term “solid support” as used herein refers to a material having a rigid or semi-rigid surface that contains or can be derivatized to contain reactive functionalities that covalently link a compound to the material's surface. Such materials are well known in the art and include, by way of example, silicon dioxide supports containing reactive Si—OH groups, polyacrylamide supports, polystyrene supports, polyethylene glycol supports, and the like. Such supports will preferably take the form of small beads, pellets, disks or other conventional forms, although other forms may be used. Preferred substrates include polystyrene resins.

The preferred synthesis of the compounds of the invention using a solid support is as follows. Initially, a protected diamine is coupled to a solid support through a cleavable linkage; the support-bound diamine may be represented as

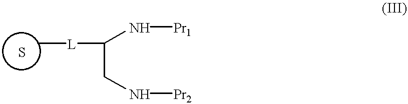

wherein “S” represents the solid support, L is a cleavable linking group such as an ester or amide linkage, and Pr₁ and Pr₂ are orthogonally removable protecting groups that can both be removed without affecting the linker L. Generally, although not necessarily, Pr₁ represents an acid-labile protecting group and Pr₂ is an acid-stable protecting group.

For example, a di-N-protected diaminopropionic acid—wherein one amine group is protected with Pr₁ (e.g., Boc (t-butoxycarbonyl)) and the second amine group is protected with P₂ (e.g., Fmoc (fluorenylmethyl oxycarbonyl))—may be coupled to a solid support having surface hydroxyl groups, through an ester linkage, as follows:

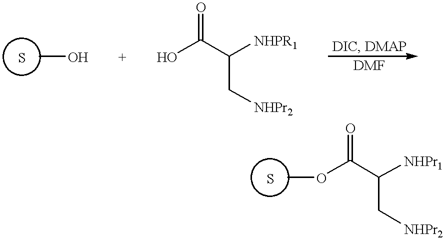

(See Example 1, part (a).) On one of the amino groups, a nitrogen-bound hydrogen atom is then replaced with an allyl moiety using a Mitsunobu reaction or an alternative allylation reaction; the support-bound product so produced may be represented structurally as follows:

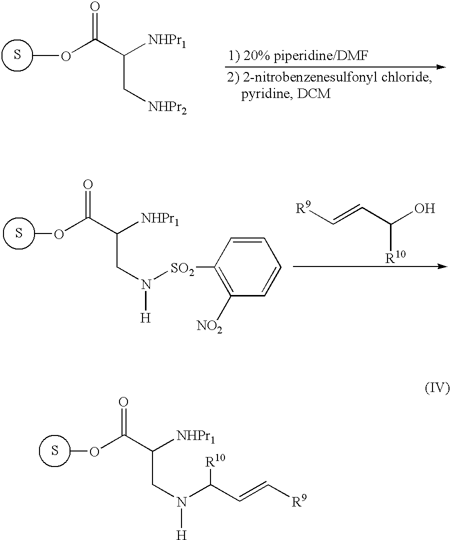

In compound (IV), the nitrophenylsulfonyl moiety is represented as “Pr₃,” a group that is orthogonally removable vis-a-vis Pr₁. In the next step of the synthesis, the protecting group Pr₁ is removed, typically with acid, and the support-bound compound so provided is then treated with an aldehyde R³—(L₂)ₙ—CHO wherein R³, L² and n are as defined elsewhere herein, providing a support-bound imine analog (V)

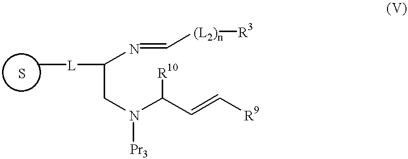

Cyclization is then effected using suitable reagents, giving rise to a supported bicyclic pyrrolidine (VI):

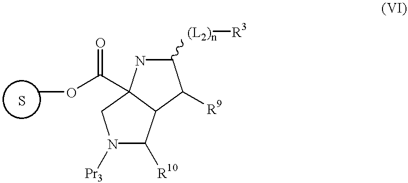

An amino derivative H₂N—(L₁)ₘ—X(R¹R²) (wherein L₁, m, R¹ and R² are as defined earlier) is then coupled through a urea or thiourea linkage (using phosgene or thiophosgene, respectively) to the free nitrogen atom on the bicyclic pyrolidine structure to produce (VII)

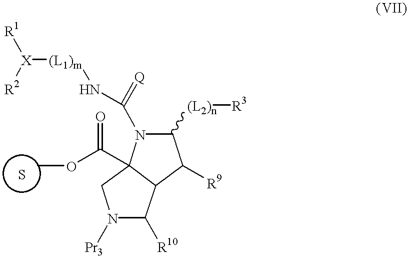

In the final step of the reaction, Pr₃ is removed and an aromatic aldehyde having the structure

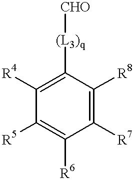

is coupled to that ring nitrogen using a reductive alkylation reaction. In this latter step, the aromatic aldehyde shown may be replaced with an identical compound bearing a bromomethyl sub stituent or an alternative leaving group adjacent to (L₃)_{q} instead of the aldehyde moiety. At this point, a bicyclic pyrrolidine derivative is produced that is still support bound. The cleavable linkage L may be cleaved, releasing the compound. Alternatively, further cyclization may be conducted so as to convert the compound to a tricyclic pyrrolidine derivative; typically, this is done with an RO⁻M⁺ moiety (where M⁺ is a cationic counterion) such as tBuO⁻K⁺, which simultaneously releases the tricyclic compound from the solid support. This is shown schematically as follows:

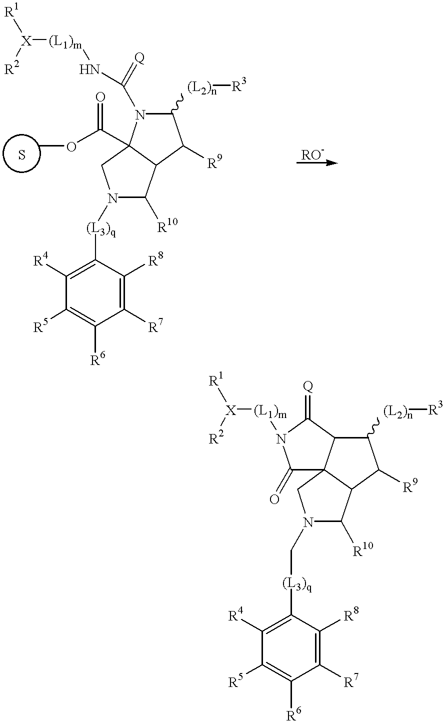

A preferred unsupported synthesis begins with a di-N-protected diamino carboxylic acid, which may be represented as (XI)

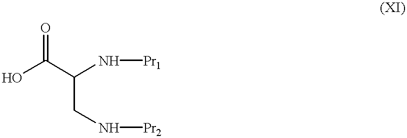

wherein Pr₁ and Pr₂ are orthogonally removable protecting groups. Generally, although not necessarily, Pr₁ represents an acid-labile protecting group and Pr₂ is an acid-stable protecting group as discussed before with respect to the supported synthesis method.

In the first step of the synthesis, the acid moiety is converted to an ester group using conventional esterification procedures, e.g., the carboxylic compound (XI) may be converted to an acetate moiety using dimethylaminopyradine in methanol followed by dichloromethane in HCl, as show below wherein R is lower alkyl, preferably methyl:

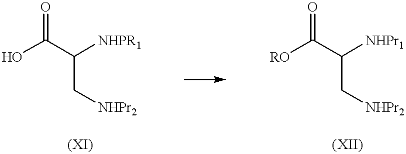

(See Example 10, part (b).) On one of the amino groups, a nitrogen-bound hydrogen atom is then replaced with an allyl moiety using a Mitsunobu reaction or an alternative allylation reaction; the support-bound product so produced may be represented structurally as follows:

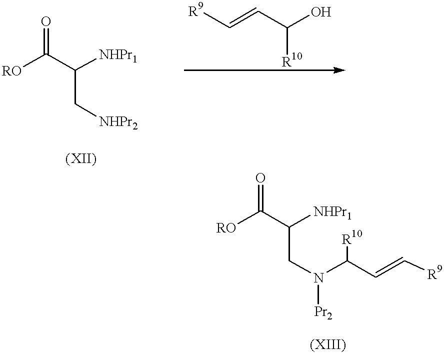

In the next step of the synthesis, the protecting group Pr, is removed, typically with acid, and the compound so provided is treated with an aldehyde, R³—(L₂)ₙ—CHO, wherein R³, L² and n are as defined elsewhere herein, providing an imine analog (XIV)

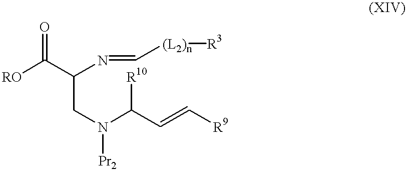

Cyclization is then effected using suitable reagents, giving rise to the bicyclic pyrrolidine (XV):

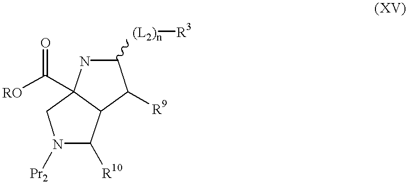

An amino derivative H₂N-(L₁)ₘ—X(R¹R²) (wherein L₁, m, R¹ and R² are as defined earlier) is then coupled through a urea or thiourea linkage (using phosgene or thiophosgene, respectively) to the free nitrogen atom on the bicyclic pyrrolidine structure. In a preferred embodiment, as the amino derivative is coupled to the growing compound, further cyclization is conducted, converting the compound to a tricyclic pyrrolidine having the structure (XVI)

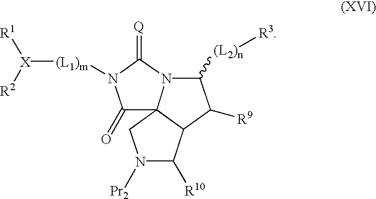

As before, in the final step of the reaction, the remaining protecting group is removed and an aromatic aldehyde having the structure

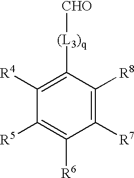

is coupled to that ring nitrogen using a reductive alkylation reaction. As indicated before, R³ through R⁸ may be substituted with various reactive moieties, i.e., hydroxyl, halogen, amino, amido, nitro, nitrile, substituted amino, sulfato, etc. Further modification of these moieties is possible both during and after synthesis of the compound

### Experimental

The following examples are put forth so as to provide those of ordinary skill in the art with a complete disclosure and description of how to prepare and use the compounds disclosed and claimed herein. Efforts have been made to ensure accuracy with respect to numbers (e.g., amounts, temperature, etc.) but some errors and deviations should be accounted for. Unless indicated otherwise, parts are parts by weight, temperature is in ° C. and pressure is at or near atmospheric.

The resin used (Merrifield) was obtained commercially available from Nova Biochem. Solid phase reactions were carried out at room temperature. Unless otherwise indicated, all starting materials and reagents were obtained commercially, e.g., from Aldrich, Sigma and ICN, and used without further purification.

Also, in these examples, unless otherwise stated, the abbreviations employed have their generally accepted meanings, as follows:

Boc = t-butoxycarbonyl
DBU = 1,3-diazabicyclo[5.4.0]undec-7-ene
DCM = dichloromethane
DEAD = diethyl azodicarboxylate
DIAD = diisopropyl diazodicarboxylate
DIC = (diethylamino)isopropyl chloride hydrochloride
DIEA = diethylamine
DMAP = dimethylamino pyridine
DMF = dimethyl formamide
EDC1 = ethylene dichloride
EDIA = diisopropyl ethylamine
eq. = equivalent(s)
Fmoc = fluorenylmethyl oxycarbonyl
g = gram
MeOH = methanol
mL = milliliter
mmol = millimole
NMP = N-methyl pyrrolidone
pip = piperidine
TFA = trifluoroacetic acid
TLC = thin layer chromatography

All patents, patent applications, journal articles and other references mentioned herein are incorporated by reference in their entireties.

### EXAMPLE 1

This example describes synthesis of “AF21276,” a tricyclic pyrrolidine hydantoin having the structural formula

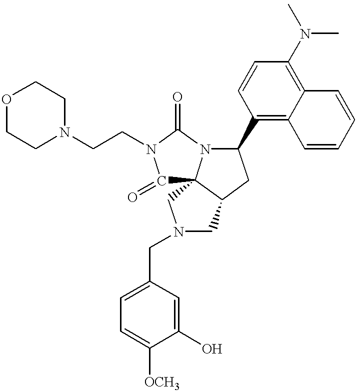

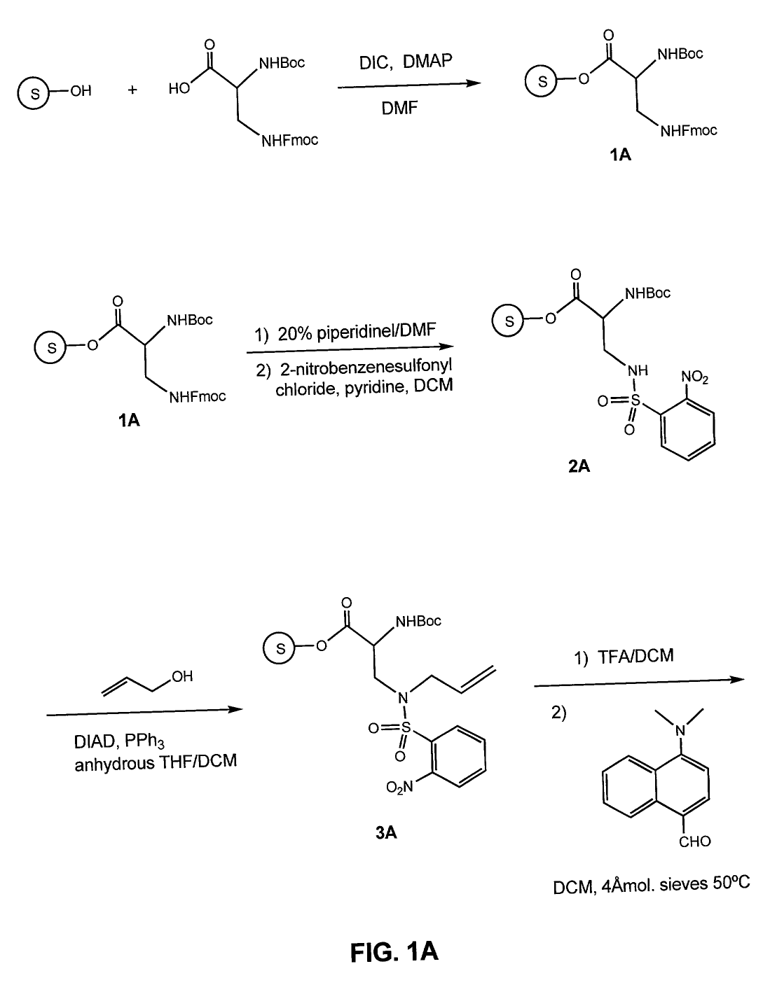

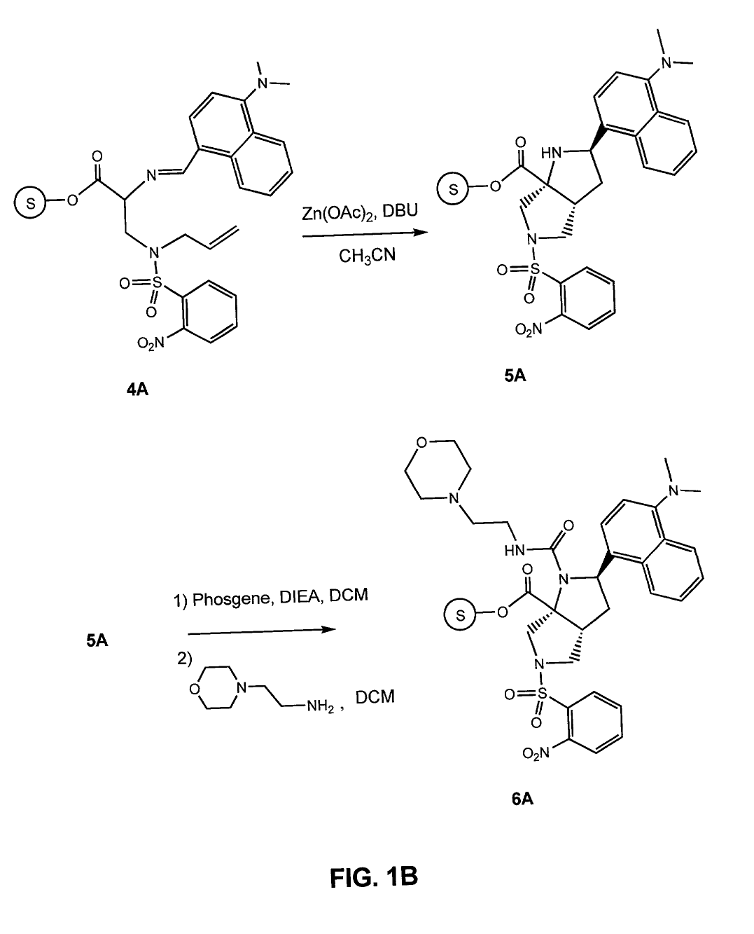

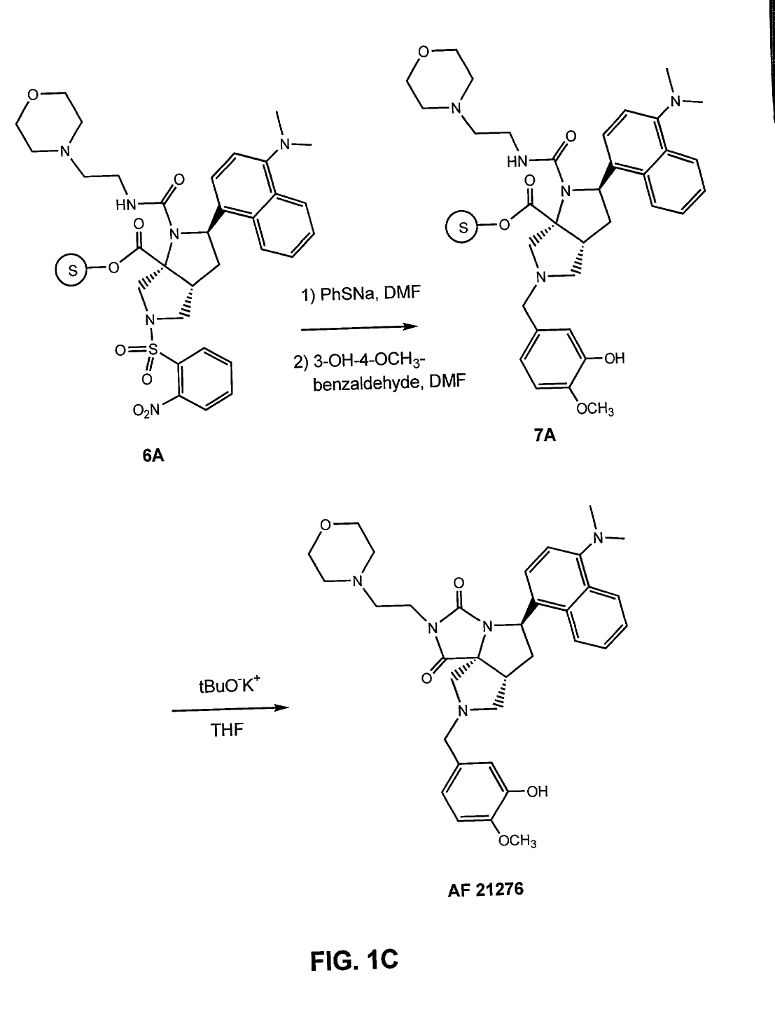

The synthetic method follows that shown schematically in FIGS. 1A, 1B and 1C.

(a) Preparation of Support-bound α-Boc-β-Fmoc-diaminopropionic Acid (Structure 1A in FIG. 1A):

To 1 gram polystyrene alcohol resin (loading 1.0 mmol/g) was added 7 eq α-Boc-β-Fmoc-diaminopropionic acid, and 10 ml DMF was then added to dissolve the amino acid. 7 eq. DIC was added followed by 0.2 eq. DMAP. The resin was shaken at room temperature for 5 h, drained, washed with DMF, MeOH, DCM and ether, and then dried in vacuo. Resin loading was measured via a standard Fmoc-determination (loading ˜0.68 mmol/g).

(b) Formation of the Support-bound N-(2-nitrobenzenesulfonyl) Derivative (Structure 2A in FIG. 1A):

1 g of 1A was shaken for 30 min. with 15 ml 20% piperidine/DMF solution, and the resin was then filtered and washed with DMF, MeOH and DCM. A standard ninhydrin test showed a deep blue color. To the resin beads were then added 13.6 ml DCM followed by 11 eq. pyridine and 10 eq. of 2-nitrobenzenesulfonyl chloride (powder). After shaking at room temperature overnight the resin beads were washed with DMF, MeOH and DCM. A standard ninhydrin test was negative.

(c) Synthesis of the Support-bound N-allyl Derivative (Structure 3A in FIG. 1A) via a Mitsunobu Reaction:

1 g of 2A was placed in a glass vial, to which was added 20 ml of 1:1 (v:v) anhydrous THF/DCM followed by 20 eq. of triphenyl phosphine (in solid form). 20 eq. of allyl alcohol was added. After the two reagents were dissolved the reaction mixture was cooled to 0° C. under argon. 20 eq. of diisopropyl azodicarboxylate in 3 ml of 1:1 (v:v) anhydrous THF/DCM solvent was added dropwise. After addition was complete, the reaction vial was agitated for 3 h at room temperature. The resin beads were then washed with DMF, MeOH and DCM. (All the solvents used in this reaction must be anhydrous. DEAD may, if desired, be substituted for the DIAD in the Mitsunobu reaction; however, it is critical that this reaction take place under strictly maintained anhydrous conditions.

(d) Formation of the Support-bound Imine (Structure 4A in FIG. 1B):

15 ml of 50% TFA in DCM was added to 1 g of 3A, the reaction vessel was agitated for 1 h at room temperature, and the resin beads were then washed with DCM, 0.2 M ammonia/MeOH/DCM solution, then DCM. 15 eq. 4-dimethylamino-1-naphthaldehyde (in solid form) was added, followed by 10 ml DCM and fresh molecular sieves. The reaction mixture was shaken at 50-55° C. in a sand bath overnight. The beads were washed with DCM.

(e) Cyclization to Give Support-bound Bicyclic Pyrrolidine (Structure 5A in FIG. 1B):

To 1 g of 4A was added 10 eq. zinc acetate, followed by 13.6 ml of acetonitrile and 10 eq. DBU. After shaking at room temperature overnight, the beads were washed with DMF, MeOH and DCM.

(f) Attachment of the Morpholino Functionality (Structure 6A in FIG. 1B):

To 1 g of compound 5A, prepared as described in the preceding section, in a glass vial was added 6 ml DCM and 20 eq. DIEA. The reaction mixture was cooled to 0° C. under argon, and 20 eq. of phosgene solution (1.93 M in toluene from Fluka) was added dropwise. After addition was complete, the reaction mixture was agitated for 1 h at room temperature. The beads were then washed with DCM. To the resin was added 13.8 ml of DCM followed by 20 eq. 4-(2-aminoethyl)morpholine. After shaking at room temperature for 1 h the beads were washed with DCM and DMF.

(g) Preparation of the N-(3-hydroxy-4-methoxy-benzaldehyde) Derivative (Structure 7A in FIG. 1C):

To 1 g of 6A, prepared as described in the preceding section, was added 6.8 ml of DMF followed by 6.8 ml of a 1 M solution of PhSNa in DMF. After shaking at room temperature for 1 h, the beads were washed with DMF, MeOH, DCM and DMF. This procedure was repeated for another 1 h. 20 eq. of 3-hydroxy-4-methoxy-benzaldehyde was added, followed by 6.8 ml of DMF and 2.7 ml of AcOH. After shaking at room temperature for 15 min, 20 eq. Na(OAc)₃BH in 6.8 ml NMP solution was added, and the resin was agitated at room temperature for 1 h. The beads were washed with MeOH, DMF, NMP, DCM and ether, then thoroughly dried in vacuo for 24 h.

(h) Preparation and Release of the Product (Compound AF 21276 in FIG. 1C):

To 1 g of 7A was added 5 eq. of tBuOK powder followed by 10 ml anhydrous THF. After shaking at room temperature for 1 h, the solution was filtered and the beads were washed with THF. The combined THF solutions were neutralized using a cation exchange resin (Dowex HCR-W2, H⁺ Form, 16-40 Mesh, about 50 mg). After shaking for 1 h, the resin was filtered off, and the THF evaporated. The reaction product was purified by preparative TLC using 40% EtOAc/hexanes as eluant, and isolated in ˜25% overall yield (based on initial resin loading). MS for Compound AF21276: found, 600 (M+H⁺). ¹H NMR: 8.36 (d, 1H, J=8.8 Hz), 8.30 (d, 1H, J=8.8 Hz), 7.62 (t, 1H, J=6.6 Hz), 7.54 (t, 1H, J=6.6 Hz), 7.27 (d, 1H, J=8.8 Hz), 7.01 (d, 1H, J=8.1 Hz), 7.00 (s, 1H), 6.86 (d, 1H, J=8.1 Hz), 6.81(d, 1H, J=8.1 Hz), 5.77 (dd, 1H, J=4.4 Hz, 12.5 Hz), 3.89 (s, 3H), 3.65(d, 1H, J=12.8 Hz), 3.58 (t, 4H, J=4.4 Hz), 3.53 (d, 1H, J=12.8 Hz), 3.43(t, 2H, J=6.6 Hz), 3.28(d, 1H, J=10.2 Hz), 3.13 (t, 1H, J=8.1 Hz), 2.86-2.96 (m, 2H), 2.91 (s, 6H), 2.78 (t, 1H, J=8.8 Hz), 2.62 (dd, 1 H, J=8.8 Hz, 12.5 Hz), 2.37-2.47 (m, 6H), 2.15 (dd, 1H, J=4.4 Hz, 12.5 Hz).

### EXAMPLE 2

The procedure of Example 1 was repeated, but 3-ethoxy-4-hydroxy-benzaldehyde was substituted for 3-hydroxy-4-methoxy-benzaldehyde in step (g). An active GnRH antagonist was produced having the structural formula

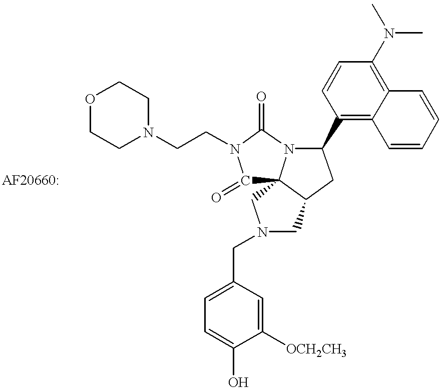

MS for Compound AF20660: found, 614 (M+H⁺). ¹H NMR: 8.28-8.33 (m, 2H), 7.52-7.55 (m, 2H), 7.28 (d, 1H, J=8.0 Hz), 7.01 (d, 1H, J=8.0 Hz), 6.91 (s, 1H), 6.83-6.89 (m, 2H), 5.74 (dd, 1H, J=3.3 Hz, 12.1 Hz), 4.02 (q, 1H, J=7.0 Hz), 3.95 (q, 1H, J=7.0 Hz), 3.66 (d, 1H, J=12.8 Hz), 3.58 (t, 4H, J=4.4 Hz), 3.52 (d, 1H, J=12.8 Hz), 3.43 (t, 2H, J=6.2 Hz), 3.27 (d, 1H, J=10.3 Hz), 3.13 (t, 1H, J=6.6 Hz), 2.94 (t, 2H, J=9.5 Hz), 2.91 (s, 6H), 2.79 (t, 11H, J=9.2 Hz), 2.62 (q, 1H, J=12.1 Hz), 2.36-2.47 (m, 6H), 2.16 (dd, 1H, J=4.0 Hz, 12.8 Hz), 1.31 (t, 3H, J=7.0 Hz).

### EXAMPLE 3

The procedure of Example 1 was repeated, but 2,3-dibromo-4-hydroxy-5-methoxy-benzaldehyde was substituted for 3-hydroxy-4-methoxy-benzaldehyde in step (g). An active GnRH antagonist was produced having the structural formula

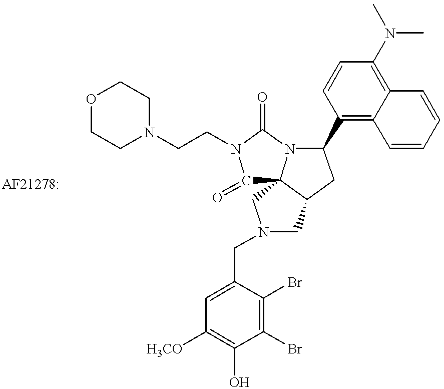

MS for Compound AF21278: found, 758 (M+H⁺). ¹H NMR: 8.27-8.29 (m, 2H), 7.50-7.54 (m, 2H), 7.26-7.28 (m, 1H), 7.05 (s, 1H), 6.99 (d, 1H, J=8.0 Hz), 5.70 (dd, 1H, J=4.4 Hz, 12.8 Hz), 3.76-3.82 (m, 4H), 3.58 (t, 4H, J=4.4 Hz), 3.45 (td, 1H, J=1.8 Hz, 6.6 Hz), 3.39 (t, 1H, J=7.3 Hz), 3.25 (d, 1H, J=10.3 Hz), 3.17 (t, 1H, J=7.3 Hz), 3.08 (d, 1H, J=10.3 Hz), 2.92-3.10 (m, 1H), 2.91 (s, 6H), 2.59-2.67 (m, 1H), 2.36-2.48 (m, 6H), 2.16 (dd, 1H, J=4.4 Hz, 12.5 Hz), 1.99-2.06 (m, 2H).

### EXAMPLE 4

The procedure of Example 1 was repeated, but 2-bromo-4-methoxy-5-hydroxy-benzaldehyde was substituted for 3-hydroxy-4-methoxy-benzaldehyde in step (g). An active GnRH antagonist was produced having the structural formula

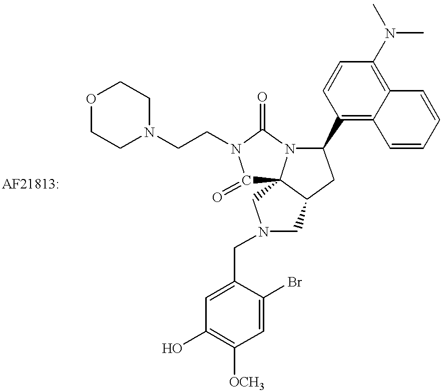

MS for Compound AF21813: found, 679 (M+H⁺). ¹H NMR: 8.33 (d, 1H, J=8.3 Hz), 8.27 (d, 1H, J=8.3 Hz), 7.49-7.59 (m, 2H), 7.24 (d, 1H, J=8.1 Hz), 6.95-7.06 (m, 3H), 5.78 (dd, 1H, J=4.4 Hz, 12.6 Hz), 3.89 (s, 3H), 3.63-3.77 (m, 2H), 3.58 (t, 4H, J=4.4 Hz), 3.43 (t, 2H, J=5.9 Hz), 3.34 (t, 1H, J=8.4 Hz), 3.15 (t, 1H, J=8.1 Hz), 2.94-3.02 (m, 2H), 2.91 (s, 6H), 2.81 (t, 1H, J=8.1 Hz), 2.54-2.65 (m, 1H), 2.34-2.48 (m, 6H), 2.12 (dd, 1H, J=4.4 Hz, 12.6 Hz).

### EXAMPLE 5

The procedure of Example 1 was repeated, except that 4-methoxy-1-naphthaldehyde was substituted for 4-dimethylamino-1-naphthaldehyde in part (d). An active GnRH antagonist was provided having the structure

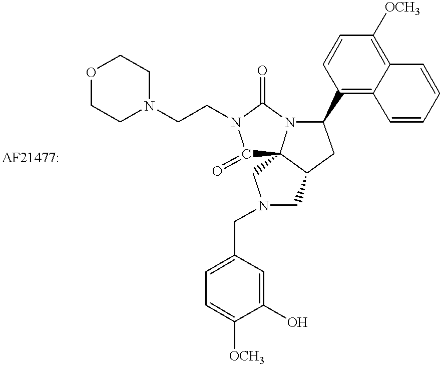

MS for Compound AF21477: found, 587 (M+H⁺). ¹H NMR: 8.31-8.34 (m, 2H), 7.65 (t, 1H, J=8.4 Hz), 7.52 (t, 1H, J=8.4 Hz), 7.27 (d, 1H, J=8.1 Hz), 7.01 (d, 1H, J=1.5 Hz), 6.86 (dd, 1H, J=1.8 Hz, 8.1 Hz), 6.80 (d, 1H, J=8.1 Hz), 6.75 (d, 1H, J=7.7 Hz), 5.77 (dd, 1H, J=4.4 Hz, 12.5 Hz), 4.00 (s, 3H), 3.89 (s, 3H), 3.66 (d, 1H, J=12.8 Hz), 3.57 (t, 4H, J=4.4 Hz), 3.54 (d, 1H, J=12.8 Hz), 3.42 (t, 2H, J=6.6 Hz), 3.27 (d, 1H, J=10.3 Hz), 3.13 (t, 1H, J=8.1 Hz), 2.97 (d, 1H, J=9.5 Hz), 2.90 (d, 1H, J=9.8 Hz), 2.78 (t, 1H, J=8.8 Hz), 2.57-2.66 (m, 1H), 2.36-2.46 (m, 6H), 2.17 (dd, 1H, J=4.4 Hz, 12.1 Hz)

### EXAMPLE 6

The procedure of Example 1 was repeated, except that 4-dimethylaminobenzaldehyde was substituted for 4-dimethylamino-1-naphthaldehyde in part (d). An active GnRH antagonist was provided having the structure

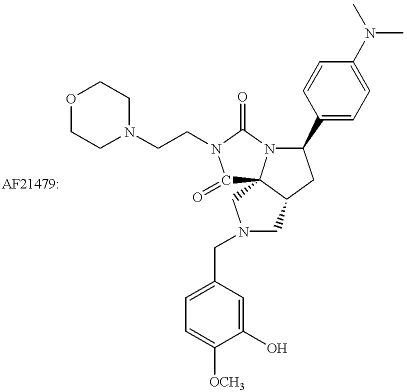

MS for Compound AF21479: found, 550 (M+H⁺). ¹H NMR: 7.31-7.25 (m, 2H), 6.94 (s, 1H), 6.81 (s, 2H), 6.70 (d, 2H, J=8.8 Hz), 5.01 (dd, 1H, J=5.7 Hz, 11.1 Hz), 3.89 (s, 3H), 3.51-3.65 (m, 8H), 3.09 (t, 1H, J=9.9 Hz), 3.00-3.04 (m, 1H), 2.95 (s, 6H), 2.86-2.91 (m, 1H), 2.85 (d, 1H, J=7.8 Hz), 2.65 (t, 1H, J=8.0 Hz), 2.35-2.51 (m, 7H), 2.05-2.13 (m, 1H).

### EXAMPLE 7

The procedure of Example 1 was repeated, except that 4-(2-aminoethyl)piperazine was substituted for 4-(2-aminoethyl)morpholine. An active GnRH antagonist was provided, having the structural formula

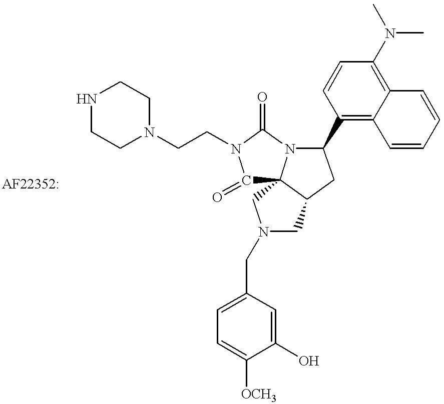

MS for Compound AF22352: found, 599 (M+H⁺). ¹H NMR: 8.31 (t, 2H, J=9.9 Hz), 7.62 (t, 1H, J=7.0 Hz), 7.53 (t, 1H, J=7.3 Hz), 7.27 (d, 1H, J=7.6 Hz), 6.99-7.03 (m, 2H), 6.83 (dd, 2H, J=8.4 Hz, 13.9 Hz), 5.75 (dd, 1H, J=4.4 Hz, 12.1 Hz), 3.89 (s, 3H), 3.74 (s, 1H), 3.69 (d, 1H, J=12.4 Hz), 3.51 (d, 1H, J=12.8 Hz), 3.35-3.48 (m, 2H), 3.22 (d, 1H, J=10.2 Hz), 3.13 (t, 1H, J=9.1 Hz), 2.93 (t, 1H, J=9.1 Hz), 2.91 (s, 6H), 2.81 (t, 1H, J=9.1 Hz), 2.54-2.74 (m, 8H), 2.38-2.49 (m, 4H), 2.17(dd, 1H, J=4.4 Hz, 12.8 Hz).

### EXAMPLE 8

The procedure of Example 1 was repeated, except that 4-(2-aminoethyl)pyridine was substituted for 4-(2-aminoethyl)morpholine. An active GnRH antagonist was provided, having the structural formula

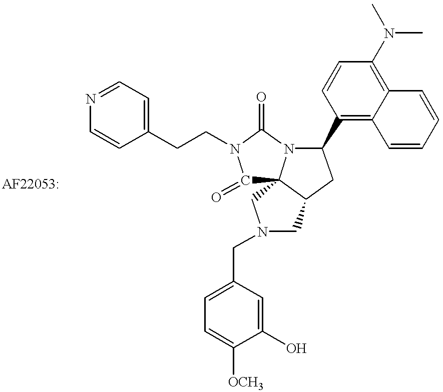

MS for Compound AF22053: found, 592 (M+H⁺). ¹H NMR: 8.47 (dd, 2H, J=1.8 Hz, 4.4 Hz), 8.29 (t, 2H, J=6.7 Hz), 7.62 (t, 1H, J=8.4 Hz), 7.53 (t, 1H, J=8.0 Hz), 7.20 (d, 1H, J=7.7 Hz), 7.00-7.05 (m, 3H), 6.96 (d, 1H, J=1.8 Hz), 6.81-6.85 (m, 2H), 5.72 (dd, 1H, J=4.4 Hz, 12.1 Hz), 3.89 (s, 3H), 3.52-3.64 (m, 4H), 3.17 (d, 1H, J=10.3 Hz), 2.97 (t, 1H, J=7.7 Hz), 2.92 (s, 7H), 2.79-2.83 (m, 3H), 2.73 (t, 1H, J=8.0 Hz), 2.40-2.43 (m, 1H), 2.09 (dd, 1H, J=4.4 Hz, 12.4 Hz).

### EXAMPLE 9

### Optimization of the Stereochemistry of the Cycloaddition Reaction

The procedure of part (d) of Example 1 was arrived at in an attempt to optimize the preparation of the “syn” isomer shown below (i.e., in the “syn” isomer, the dimethylaminonaphthalenyl group is “syn” with respect to the tricyclic center), as that isomer has been found to be the more potent GnRH antagonist.

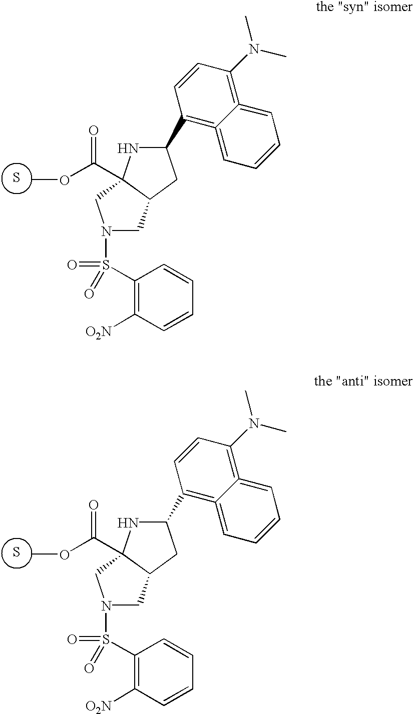

Reaction conditions explored gave different ratios of the “syn” and “anti” isomers as shown in Table 1:

TABLE 1

| Reaction Conditions | “syn”: “anti” ratio |
|---|---|
| AcOH, DCM, 60° C. (Ex. 1) | 1:2.3 |
| AcOH, DMF, 60° C. | 1:0.58 |
| AcOH, DMF, 80° C. | 1:0.66 |
| AcOH, CH₃CN, 60° C. | 1:1.59 |
| AcOH, CH₃CN, 80° C. C. | 1:2.45 |
| 2,4-dinitrophenol, DCM, 60° C. | 1:2.21 |
| AgNO₃, CH₃CN, 60° C. | 1:0.76 |
| Zn(OAc)₂, CH₃CN, 60° C. | 1:0.23 |
| LiBr, THF, 60° C. | 1:0.33 |
| DCM, 60° C. | 1:0 |

It was found that combining an initial DCM/60° C. treatment with subsequent exposure to Zn(OAc)₂/DBU in CH₃CN provides clean conversion of the starting material to the desired product, and introducing this step into the procedure of Example 1 provides AF 21276 as the syn isomer in approximately 25% isolated yield.

### EXAMPLE 10

This example describes the unsupported synthesis of “8” a tricyclic pyrrolidone hydantoin having the structural formula

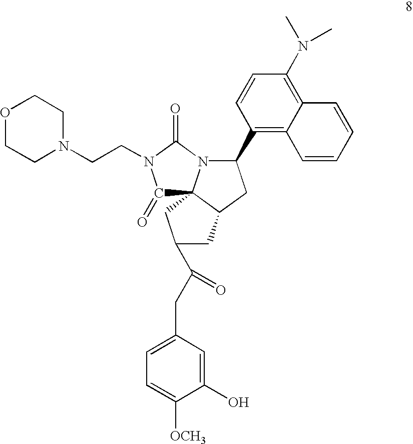

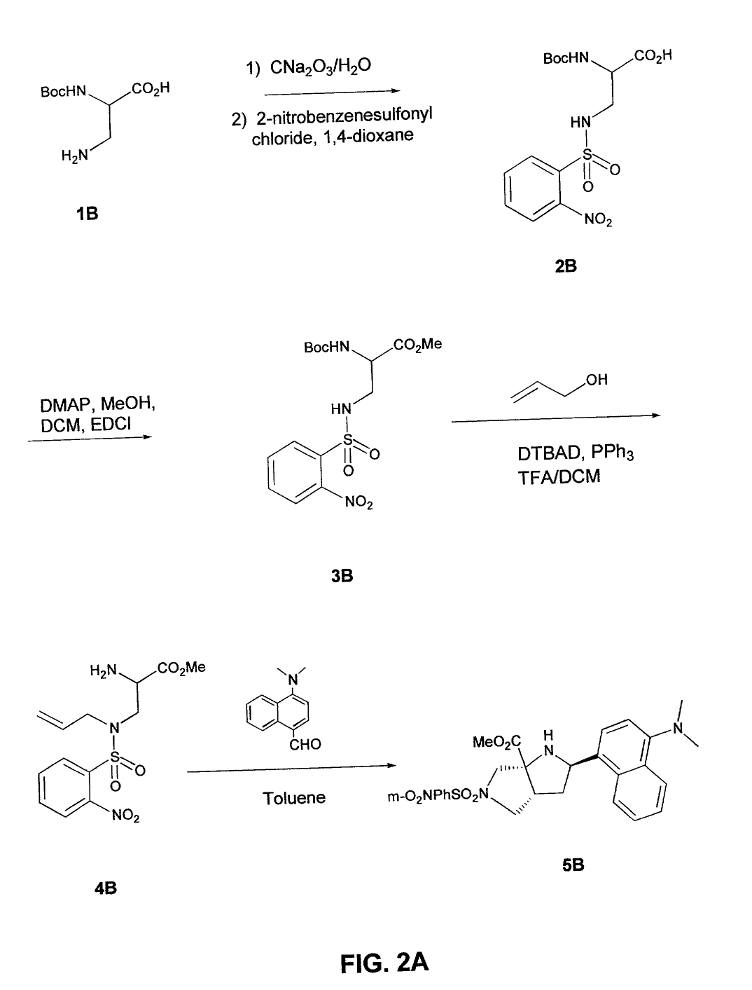

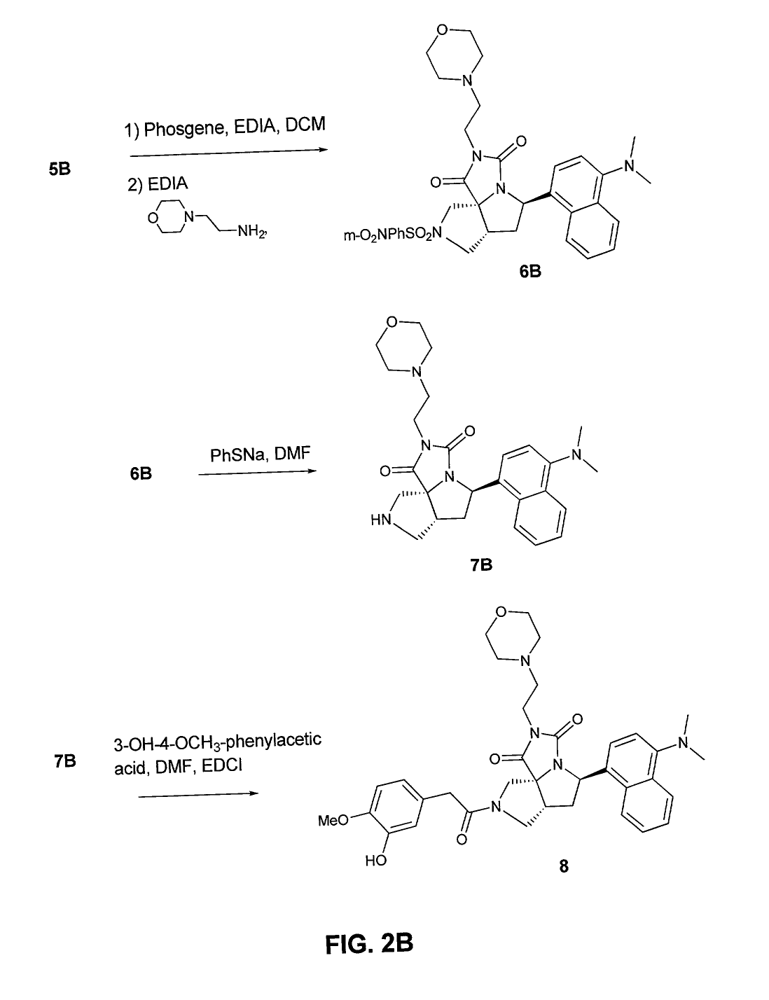

The synthetic method follows that shown schematically in FIGS. 2A and 2B.

(a) Preparation of (2S)-2-[(tert-Butoxycarbonyl)amino]-3-{[(2-nitrophenyl)sulfonyl]amino}propionic Acid (Structure 2B in FIG. 2A):

To a solution of sodium carbonate (5.3 g, 50 mMmol) in water (125 mL) was added N-α-boc-2,3-diaminopropionic acid 1B and the mixture stirred until a homogeneous solution was attained. The solution was cooled in an ice bath and treated with 2-nitrophenylsulfonyl chloride (5.5 g, 25 mmol) as well as 1,4-dioxane (125 mL). The mixture stirred for 3 hours while the temperature slowly rose to 20° C. (a colorless precipitate formed). The reaction was diluted with water (500 mL), washed with ethyl acetate (2×200 mL), neutralized with conc. HCl (pH=1) and extracted with ethyl acetate (2×150 mL). The combined organic extracts were dried (MgSO₄) and evaporated to leave 2B as a beige foamy gum (10 g, 100%). ¹H-NMR (CDCl₃); δ 8.13 (m, 1H), 7.87 (m, 1H), 7.75(m, 2H), 6.00 (bt, 1H, J=4.0 Hz), 5.50 (bd, 1H, J=4.3 Hz), 4.38 (m, 1H), 3.57 (m, 2H), 1.45 (s, 9H): LC/MS indicated 97% purity, M−H=387. Used without further purification.

(b) Synthesis of Methyl (2S)-2-[(tert-butoxycarbonyl)amino]-3-{[(2-nitrophenyl)sulfonyl]amino}propanoate (Structure 3B in FIG. 2A):

A solution of the acid 2B (10 g, 25 mMol), DMAP (0.31 g, 2.5 mMol) and methanol (1.6 g, 50 mMol, 2.0 mL) in dichloromethane (250 mL) was cooled in an ice bath and treated with EDCI (4.8 g, 25 mMol) all at once. After stirring for 45 mins. the mixture was washed with 1N HCl, water, saturated aqueous sodium bicarbonate and water. The solution was dried and evaporated to leave 3 as a viscous oil (9.0 g, 89%). ¹H-NMR (CDCl₃); δ 8.13 (m, 1H), 7.88 (m, 1H), 7.76 (m, 2H), 5.81 (bt, 1H, J=4.0 Hz), 5.33 (bd, 1H, J=4.4 Hz), 4.38 (m, 1H), 3.80 (s, 3H), 3.52 (m, 2H), 1.45 (s, 9H): LC/MS indicated 96% purity, M-H 402. Used without further purification.

(c) Formation of the Allyl Amine Derivative, Methyl 3-{allyl[(2-nitrophenyl)sulfonyl]amino}-2-aminopropanoate (Structure 4B if FIG. 2A):

To a mixture of the sulfonamide 3B (5.0 g, 12 mMol), allyl alcohol (0.86 g, 15 mMol, 1.0 mL) and polymer supported triphenylphosphine (10 g, 3.0 mMol/g, 30 mMol) in dichloromethane (150 mL) at ice temperature was added di-t-butylazodicarboxylate (4.1 g, 18 mMol) and the mixture stirred slowly for 1.5 h. The ice bath was removed and trifluoroacetic acid (75 mL) was added. Stirring continued for 1 h and the mixture was filtered (celite), washed (DCM, 2×100 mL) and the filtrate was evaporated to dryness. The residue was dissolved in ethyl acetate (150 mL) and washed with 1M sodium carbonate. The organic layer was dried (MgSO₄) and evaporated to leave 4B as a brown gum (3.3 g, 80%). ¹H-NMR (CDCl₃); δ 8.09 (m, 1H), 7.70 (m, 3H), 5.68 (m, 1H), 5.21 (m, 2H), 4.03 (m, 2H), 3.75 (m, 1H), 3.73 (s, 3H), 3.63 (dd, 1H, J=15.7 Hz, J=5.3 Hz), 3.50 (dd, 1H, J=14.7 Hz, J=8.4): LC/Ms indicated 93% purity, M+H=344. Used without further purification.

(d) Cyclization and Addition of the Napthyl Moiety to Form (trans)-methyl-2-[4-(dimethylamino)-1-naphthyl]-5-[(2-nitrophenyl)sulfonyl]hexahydropyrrolo[3,4-b]pyrrole-6a(1H)-carboxylate (Structure 5B in FIG. 2A):

A solution of the amine 4B (2.8 g, 8.2 mMol) and 4-(dimethylamino)-1-naphthaldehyde (2.1 g, 11 mMol) in toluene (84 mL) was heated to reflux for 90 mins and allowed to stand for 16 h at 20° C. The precipitated product was filtered, washed (2:1 hexane-ethyl acetate) and dried to leave the pyrrolidine 5B as a light yellow solid (2.7 g, 67%). ¹H-NMR (CDCl₃); δ 8.27 (m, 1H), 8.08 (m, 2H), 7.72 (m, 2H), 7.68 (m, 1H), 7.48 (m, 3H), 7.02 (d, 1H, J=7.8 Hz), 5.11 (dd, 1H, J=9.8 Hz, J=5.7 Hz), 4.00 (d, 1H, J=11 Hz), 3.86 (dd, 1H, J=10.2 Hz, J=8.6 Hz), 3.72 (s, 3H), 3.68 (d, 1H, J=11.0 Hz), 3.57 (dd, 1H, J=10.4 Hz, J=4.6 Hz), 3.23 (m, 1H), 2.88 (s, 6H), 2.21 (m, 2H): LC/Ms indicated >99% purity, M+H=525. Used without further purification.

(e) Attachment of the Morpholino Functionality to Form (trans)-5-[4-(Dimethylamino)-1-naphthyl]-8-(2-nitrophenylsulfonyl)-2-(2-morpholin-4-ylethyl)hexahydro-1H-pyrrolo [3′,4′:2,3]pyrrolo [1,2-c]imidazole-1,3(2H)-dione (Structure 6B in FIG. 2B):

To a solution of the pyrrolidine 5B (2.6 g, 5.0 mMol) and diisopropylethylamine (0.71 g, 5.5 mMol, 0.99 mL) in DCM (70 mL) in an ice bath under nitrogen atmosphere was added phosgene solution (1.9 M in toluene, 3.9 mL, 7.5 mMol) and the solution stirred for 90 mins. More diisopropylethylamine (3.2 g, 25 mMol, 4.5 mL) was added along with 4-(2-aminoethyl)morpholine (3.3 g, 25 mMol, 3.3 mL) and the mixture stirred an additional 30 mins. at ice temperature. The solvents were removed under vacuum, the residue was dissolved in ethyl acetate (70 mL), washed with 1M sodium carbonate solution, dried (MgSO₄), filtered and the filtrate treated with DBU (1 mL). The solution was stirred at 60° C. for 3 h, cooled to 20° C., washed with water (3×50 mL), dried (MgSO₄) and evaporated to leave 6B as a light yellow, foamy solid (2.3 g, 72%). ¹H-NMR (CDCl₃); δ 8.28 (m, 1H), 8.16 (m, 1H), 8.09 (m, 1H), 7.77 (m, 2H), 7.18 (m, 1H), 7.50 (m, 2H), 7.22 (d, 1H, J=7.7 Hz), 6.97 (d, 1H, J=7.7 Hz), 5.49 (dd, 1H, J=11.7 Hz, J=4.5 Hz), 4.12 (m, 2H), 3.90 (d, 1H, J=11.1 Hz), 3.78 (d, 2H, J=5.3 Hz), 3.60 (t, 3H, J=4.3 Hz), 3.44 (t, 2H, J=6.2 Hz) 3.28 (m, 1H), 2.90 (s, 6H), 2.70 (m, 1H), 2.40 (m, 7H): LC/MS indicated 98% purity; M+H=649. Used without further purification.

(f) Deprotection to Form (trans)-5-[4-(dimethylamino)-1-naphthyl]-2-(2-morpholin-4-ylethyl)hexahydro-1H-pyrrolo[3′,4′:2,3]pyrrolo [1,2-c]imidazole-1,3(2H)-dione (Structure 7B in FIG. 2B):

To a solution of the hydantoin 6B (0.20 g, 0.31 mMol) in DMF (2 mL) under nitrogen atmosphere was added thiophenol sodium salt (0.12 g, 0.93 mMol) and the solution stirred for 90 mins. 1N HCl Was added (20 mL) and the mixture was washed with DCM (3×15 mL), neutralized with solid potassium carbonate (pH=10) and extracted with DCM (3×20 mL). The combined organic extracts were dried (MgSO₄) and evaporated. The residue was dissolved in ethyl acetate (25 mL), washed with water (3×25 mL), dried (MgSO₄) and evaporated to leave pyrrolidine 7B as a light yellow gum (86 mg, 60%). ¹H-NMR (CDCl₃); δ 8.27 (t, 2H, J=9.5 Hz), 7.53 (m, 2H), 7.31 (d, 1H, J=7.8 Hz), 7.01 (d, 1H, J=7.8 Hz), 5.25 (dd, 1H, J=12.7 Hz, J=4.0 Hz), 3.69 (d, 1H, J=12.3 Hz), 3.61 (t, 4H, J=4.5 Hz), 3.57 (dd, 1H, J=11.7 Hz, J=3.5 Hz), 3.44 (t, 2H, J=6.4 Hz), 3.27 (d, 1H, J=12.4 Hz), 3.07 (q, 1H, J=7.5 Hz), 2.92 (s, 6H), 2.90 (m, 1H), 2.60 (m, 1H), 2.43 (m, 6H), 2.17 (dd, 1H, J=13.0 Hz, J=4.5 Hz): LC/MS indicated >99% purity; M+H=464. Used without further purification.

(g) Addition of the Benzyl Moiety to Form (trans)-5-[4-(dimethylamino)-1-naphthyl]-8-[2-(3-hydroxy-4-methoxyphenyl)acetyl]-2-(2-morpholin-4-ylethyl)hexahydro-1H-pyrrolo[3′,4′:2,3]pyrrolo[1,2-c]imidazole-1,3(2H)-dione (structure 8 in FIG. 2B):

A solution of the pyrrolidine 7B (40 mg, 86 μMol) and 3′-hydroxy-4′methoxyphenylacetic acid (17 mg, 95 μMol) in DMF (0.5 mL) was treated with EDCI (18 mg, 95 μMol) and stirred 90 mins. The mixture was diluted with ethylacetate (10 mL), washed with water (3×10 mL), dried (MgSO₄) and evaporated. The residue was chromatographed on silica gel (eluted with 5% methanol in dichloromethane) to leave 8 as a clear gum (38 mg, 70%). The product was dissolved in 1N HCl (2 mL) and evaporated to dryness at 20° C. to leave a beige, crystalline solid. ¹H-NMR (D₂O); d 8.0 (d, 1H, J=8.4 Hz), 7.88 (d, 1H, J=8.4 Hz), 7.63 (m, 3H), 7.22 (d, 1H, J=8.0 Hz), 6.69 (m, 2H), 6.62 (d, 1H, J=1.9 Hz), 6.13 (d, 1H, J=8.0 Hz), 4.44 (d, 1H, J=13.7 Hz), 4.08 (d, 1H, J=13.0 Hz), 3.92 (dd, 1H, J=12.6 Hz, J=4.2 Hz), 3.80 (dd, 1H, J=12.6 Hz, J=8.4 Hz), 3.62 (dd, 2H, J=14.1 Hz, J=10.3 Hz), 3.50 (dt, 1H, J=27.0 Hz, J=5.3 Hz), 3.50 (bm, 9H), 3.37 (t, 1H, J=6.1 Hz), 3.29 (s, 6H), 3.24 (m, 2H), 3.18 (m, 2H), 2.87 (s, 3H), 2.48 (m, 1H), 1.82 (dd, 1H, J=13.0, J=4.6 Hz): LC indicated 97% purity: MS, M+H=628.

### EXAMPLE 11

The procedure of Example 10 was repeated, except that 2-(3-Hydroxy-4-methoxyphenyl)acetaldehyde was substituted for 3′-hydroxy-4′methoxyphenylacetic acid. The 2-(3-hydroxy-4-methoxypheynyl)acetaldehyde was synthesized as described in (a) and (b) and reacted with compound 7B as described in (c).

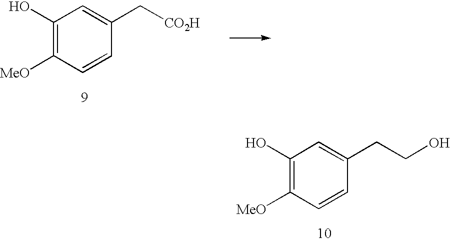

(a) Synthesis of 2-(3-Hydroxy-4-methoxyphenyl)ethanol (10):

A suspension of lithium aluminum hydride (0.42 g, 11 mmol) in THF (25 mL) under nitrogen atmosphere was cooled in an ice bath and to the mixture was added 3′-hydroxy-4′-methoxyphenylacetic acid (1.0 g, 5.5 mmol) all at once. The reaction was stirred at 20° C. for 2 h, refluxed for 1 h, cooled in an ice bath and cautiously treated with water (25 mL). After stirring an additional hour the mixture was filtered (celite) and the residue was washed with water (3×15 mL). The filtrate was treated with 1N HCl (25 mL) and extracted with ethyl acetate (3×50 mL). The combined organic layers were dried (MgSO₄) and evaporated to a crystalline solid (0.66 g, 71%). ¹H-NMR (CDCl₃); δ 6.82 (s, 1H), 6.80 (d, 1H, J=8.2 Hz), 6.71 (d, 1H, J=8.2 Hz), 5.60 (s, 1H), 3.89 (s, 3H), 3.84 (t, 2H, J=6.4 Hz), 2.80 (t, 2H, J=6.4 Hz), 1.45 (bs, 1H).

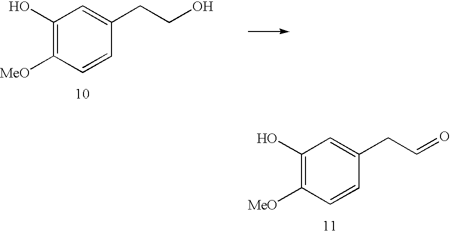

(b) Formation of 2-(3-Hydroxy-4-methoxyphenyl)acetaldehyde (11):

A solution of sulfur trioxide-pyridine complex (0.56 g, 3.6 mMol) in DMSO (4 mL) was treated with triethylamine (0.36 g, 3.6 mMol, 0.51 mL) and stirred 10 mins. The alcohol 10 (0.20 g, 1.2 mMol) was added all at once and the solution stirred 4 h. Water (20 mL) was added, the mixture stirred an additional hour and the product was extracted with ethyl acetate (3×20 mL). The combined organic extracts were washed with water (3×25 mL), dried (MgSO₄) and evaporated. The residue was chromatographed on silica gel (eluted with 1:1 hexane-ethyl acetate) to leave the aldehyde 11 as a colorless oil (63 mg, 32%). ¹H-NMR (CDCl₃); δ 9.70 (d, 1H, J 2.4 Hz), 6.84 (d, 1H, J=8.1 Hz), 6.80 (d, 1H, J=1.9 Hz), 6.70 (dd, 1H, J=8.1 Hz, J=1.9 Hz), 5.71 (s, 1H), 3.88 (s, 3H), 3.59 (d, 2H, J 2.4 Hz): LC/MS indicated 90% purity.

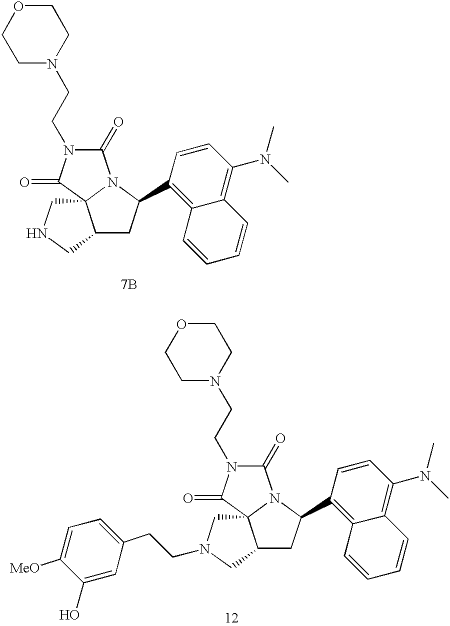

(c) Formation of (trans)-5-[4-(dimethylamino)-1-naphthyl]-8-[2-(3-hydroxy-4-methoxyphenyl)ethyl]-2-(2-morpholin-4-ylethyl)hexahydro-1H-pyrrolo[3′,4′:2,3]pyrrolo[1,2-c]imidazole-1,3(2H)-dione (12):

To a solution of the pyrrolidine 7B (0.12 g, 0.25 mMol) and the aldehyde 11 (63 mg, 0.38 mMol) in DCM (2 mL) and acetic acid (50 μL) was added sodium triacetoxyborohydride (81 mg, 0.38 mMol). The mixture stirred 1 h and was diluted with 1N HCl (10 mL) and ethyl acetate (10 mL). The separated aqueous layer was neutralized with solid sodium bicarbonate and the product was extracted with ethyl acetate (3×10 mL). The combined organic extracts were dried (MgSO₄) and evaporated. The crude product was purified by reversed phase HPLC to leave the product 12 as a glassy solid (55 mg, 36%). ¹H-NMR (CDCl₃); δ 8.27 (d, 1H, J=8.5 Hz), 8.19 (d, 1H, J=8.1 Hz), 7.52 (m, 2H), 7.25 (d, 1H, J=10.7 Hz), 6.99 (d, 1H, J=8.1 Hz), 6.80 (d, 1H, J=1.7 Hz), 6.71 (dd, 1H, J=8.1 Hz, J=1.7 Hz), 6.62 (d, 1H, J=8.1 Hz), 5.51 (bs, 1H), 5.48 (dd, 1H, J=12.0 Hz, J=4.3 Hz), 3.64 (s, 3H), 3.60 (bt, 4H, J=3.9 Hz), 3.44 (t, 2H, J=6.0 Hz), 3.28 (d, 1H, J=9.8 Hz), 3.10 (t, 1H, J=8.1 Hz), 3.00 (d, 1H, J=10.3 Hz), 2.97 (t, 1H, J=9.8 Hz), 2.91 (s, 6H), 2.88 (m, 1H), 2.76 (m, 6H), 2.60 (q, 1H, J=12.4 Hz), 2.40 (m, 4H), 2.12 (dd, 1H, J=12.0 Hz, J=4.3 Hz): ¹³C-NMR (CDCl₃); d 174.77, 157.41, 151.35, 145.52, 144.91, 134.32, 133.47, 128.76, 126.25, 125.76, 124.92, 124.72, 124.49, 123.80, 119.89, 114.68, 112.65, 110.59, 67.18, 63.41, 61.20, 60.79, 56.52, 55.77, 54.96, 53.36, 45.23, 44.77, 38.02, 35.71, 34.45, 29.71: MS; M+H=615: LC indicated >98% purity.

### EXAMPLE 12

The procedure of Example 10 was repeated, except that 4-azido-1-naphthaldehyde was substituted for 4-(dimethylamino)-1-naphthaldehyde in step (d). The remaining synthesis steps are detailed below:

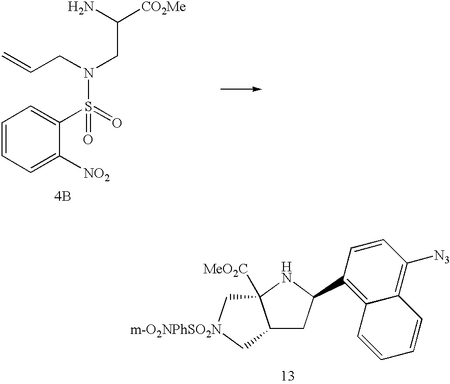

(a) Cyclization and Addition of the Naphthyl Moiety to Form (trans)-Methyl-2-[4-azido-1-naphthyl]-5-[(2-nitrophenyl)sulfonyl]hexahydropyrrolo[3,4-b]pyrrole-6a(1H)-carboxylate (13):

A solution of the amine 4B (0.50 g, 1.5 mMol) and 4-azido-1-naphthaldehyde (0.43 g, 2.2 mMol) in toluene (15 mL) was heated to 90° C. for 90 mins. The solvent was evaporated and the residue was chromatographed on silica gel (eluted with 50% hexanes in ethyl acetate) to afford the pyrrolidine 13 as a yellow gum (0.54 g, 69%). ¹H-NMR (CDCl₃); δ 8.10 (m, 3H), 7.75 (d, 1H, J 8.9 Hz), 7.73 (d, 1H, J=4.6 Hz), 7.66 (m, 2H), 7.52 (m, 2H), 7.21 (d, 1H, J=7.8 Hz), 5.21 (dd, 1H, J=9.9 Hz, J=5.6 Hz), 3.93 (d, 1H, J=11.1 Hz), 3.81 (dd, 1H, J=10.6 Hz, J=8.8 Hz), 3.73 (s, 3H), 3.71 (d, 1H, J=11.1 Hz), 3.62 (dd, 1H, J=10.6 Hz, J=4.2 Hz), 3.25 (m, 1H), 2.60 (bs, 1H), 2.32 (m, 1H): LC/MS indicated 92% purity, M+H=523.

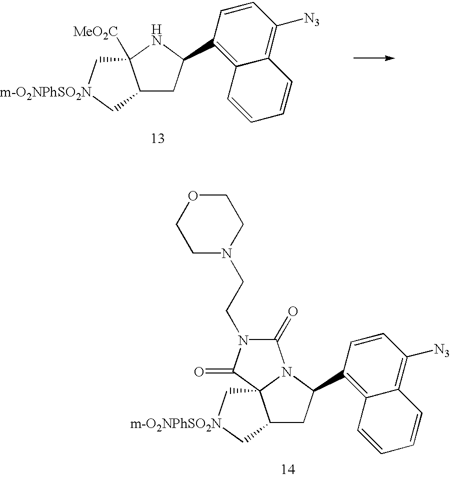

(b) Attachment of the morpholino functionality to form (trans)-5-[4-azido-1-naphthyl]-8-(2-nitrophenylsulfonyl)-2-(2-morpholin-4-ylethyl)hexahydro-1H-pyrrolo[3′,4′:2,3]pyrrolo[1,2-c]imidazole-1,3(2H)-dione (14):

To a solution of the pyrrolidine 13 (0.50 g, 0.96 mMol) and diisopropylethylamine (0.14 g, 1.1 mMol, 0.19 mL) in DCM (15 mL) under nitrogen atmosphere and ice cooling was added phosgene solution (0.76 mL of a 1.9 M solution in toluene, 1.4 mMol) dropwise over a minute. After stirring for 1 h the solution was treated sequentially with diisopropylethylamine (0.62 g, 4.8 mMol, 0.86 mL) and 4-(2-aminoethyl)morpholine (0.62 g, 4.8 mMol, 0.62 mL). After stirring for 1 h the DCM was evaporated, the residue was dissolved in ethyl acetate (50 mL) and 1M sodium carbonate solution (50 mL), separated and the organic layer was dried (MgSO₄) and evaporated to leave a foamy solid. The solid was dissolved in ethyl acetate (15 mL) and treated with DBU (0.20 mL). The mixture was heated to 60° C. for 3 h, cooled to room temperature, washed with water (3×10 mL), dried and evaporated to leave the hydantoin 14 as a brown, foamy solid (0.46 g, 74%). ¹H-NMR (CDCl₃); δ 8.18 (t, 2H, J=7.6 Hz), 8.08 (m, 1H), 7.77 (m, 2H), 7.68 (m, 1H), 7.56 (m, 2H), 7.31 (d, 1H, J=7.8 Hz), 7.19 (d, 1H, J=7.6 Hz), 5.53 (dd, 1H, J=12.0 Hz, J=4.4 Hz), 4.13 (d, 1H, J=11.1 Hz), 3.88 (d, 1H, J=11.3 Hz), 3.79 (m, 2H) 3.57 (m, 4H), 3.43 (t, 2H, J=6.1 Hz), 3.39 (m, 1H), 2.71 (m, 1H), 2.38 (m, 7H): LC/MS indicated >99% purity, M+H=647.

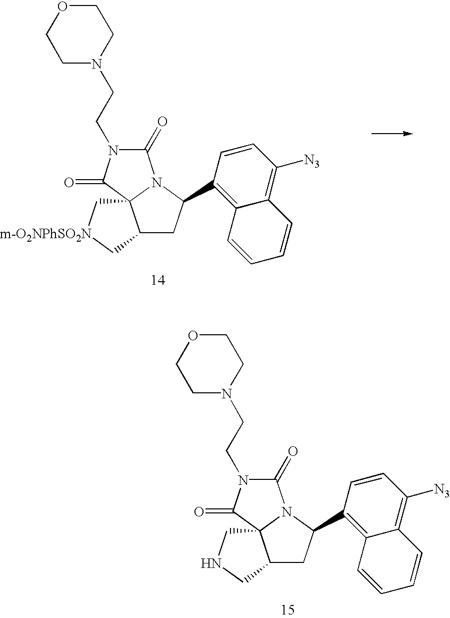

(c) Deprotection to Form (trans)-5-[4-azido-1-naphthyl]-2-(2-morpholin-4-ylethyl)hexahydro-1H-pyrrolo[3′,4′:2,3]pyrrolo[1,2-c]imidazole-1,3(2H)-dione (15):

To a solution of the hydantoin 14 (0.20 g, 0.31 mMol) in DMF (2 mL) under nitrogen atmosphere was added thiophenol sodium salt (0.12 g, 0.93 mMol). The mixture stirred 1.5 h and was then diluted with 1N HCl (20 mL). The mixture was washed with DCM (3×15 mL), neutralized with solid sodium carbonate (pH=12) and extracted with DCM (3×20 mL). The combined organic extracts were dried (MgSO₄) and evaporated. The residue, which still contained DMF, was redissolved in ethyl aceatae (25 mL), washed with water (3×20 mL), dried (MgSO₄) and evaporated to a light yellow gum (88 mg, 62%). ¹H-NMR (CDCl₃); δ 8.27 (d, 1H, J=8.3 Hz), 8.17 (d, 1H, J=8.3 Hz), 7.62 (t, 1H, J=7.0 Hz), 7.55 (t, 1H, J=8.0 Hz), 7.39 (d, 1H, J=7.8 Hz), 7.22 (d, 1H, J=7.8 Hz), 5.30 (dd, 1H, J=12.3 Hz, J=4.4 Hz), 3.68 (d, 1H, J=12.2 Hz), 3.58 (m, 5H), 3.43 (t, 2H, J=6.3 Hz), 3.27 (d, 1H, J=12.2 Hz), 3.10 (q, 1H, J=7.3 Hz), 2.93 (m, 1H), 2.60 (td, 1H, J=10.2 Hz, J=8.0 Hz), 2.44 (m, 6H), 2.20 (dd, 1H, J=10.0 Hz, J=4.4 Hz): LC/MS indicated >99% purity, M+H=462.

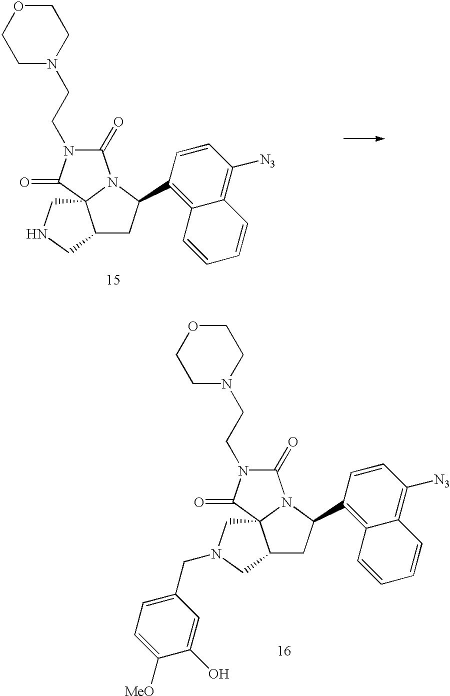

(d) Addition of the Benzyl Moiety to Form (trans)-5-[4-azido-1-naphthyl]-8-(3-hydroxy-4-methoxybenzyl)-2-(2-morpholin-4-ylethyl)hexahydro-1H-pyrrolo[3′,4′:2,3]pyrrolo[1,2-c]imidazole-1,3(2H)-dione (16):

To a solution of the pyrrolidine 15 (80 mg, 0.17 mMol) and 3-hydroxy-4-methoxybenzaldehyde (52 mg, 0.34 mMol) in DCM (1 mL) and acetic acid (0.20 mL) was added sodium triacetoxyborohydride (72 mg, 0.34 mMol). After stirring for 1 h the mixture was diluted with ethyl acetate (20 mL), washed with water (2×15 mL) and saturated aqueous sodium bicarbonate solution (15 mL), dried (MgSO₄) and evaporated. The residue was dissolved in a mixture of methanol (2 mL), trimethyl orthoformate (2 mL) and DCM (2 mL)>Aminomethylated polystyrene (2% DVB, 200-400 mesh, 0.28 g, 2.4 mMol/g, 0.68 mMol) was added to the solution and after gentle stirring for 1 h the mixture was filtered (polypropylene frit), washed with dichloromethane (2×5 mL) and evaporated to a tan gum (0.10 g, 99%). ¹H-NMR (CDCl₃); δ 8.47 (d, 1H, J=8.4 Hz), 8.17 (d, 1H, J=8.4 Hz), 7.68 (t, 1H, J=7.1 Hz), 7.5 (t, 1H, J=8.0 Hz), 7.34 (d, 1H, J=7.7 Hz), 7.21 (d, 1H, J=7.7 Hz), 7.01 (s, 1H), 6.83 (m, 2 H), 5.80 (dd, 1H, J=12.2 Hz, J=4.4 Hz), 5.15 (bs, 1H), 3.89 (s, 3H), 3.68 (d, 1H, J=12.6 Hz), 3.57 (m, 5H), 3.39 (t, 2H, J=6.4 Hz), 3.27 (d, 1H, J=9.8 Hz), 3.14 (t, 1H, J=5.5 Hz), 3.00 (d, 1H, J=10.0 Hz), 2.88 (d, 1H, J=10.0 Hz), 2.80 (t, 1H, J=6.2 Hz), 2.52 (m, 1H), 2.40 (m, 6H), 2.21 (dd, 1H, J=11.5 Hz, J=4.0 Hz): LC/MS indicated purity >99%, M+H=598.

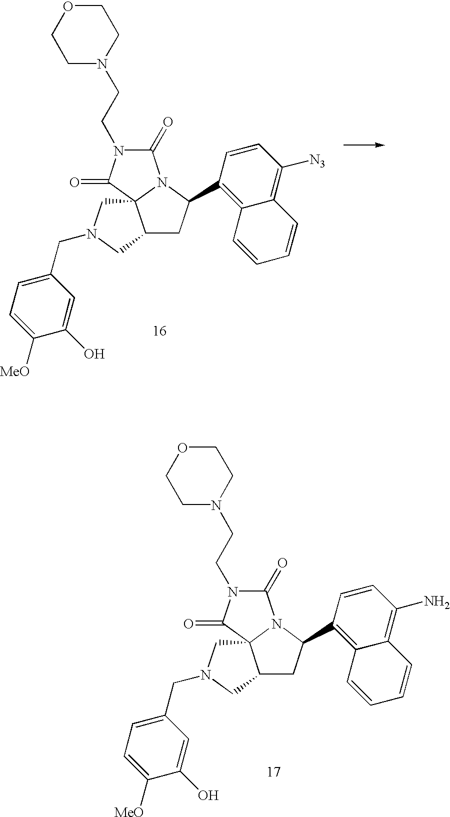

(e) Conversion of the azido moiety to form (trans)-5-[4-amino-1-naphthyl]-8-(3-hydroxy-4-methoxybenzyl)-2-(2-morpholin-4-ylethyl)hexahydro-1H-pyrrolo[3′,4′:2,3]pyrrolo[1,2-c]imidazole-1,3(2H)-dione (17):

A solution of azide 16 (0.13 g, 0.22 mMol) in THF (3 mL) under nitrogen atmosphere was treated with (n-Bu)₃P (67 mg, 0.33 mMol, 81 μL). After stirring for 2 h the solution was treated with water (0.30 mL) and heated to reflux for 16 h. The mixture was cooled to room temperature, treated with ethyl acetate (20 mL) and 1N HCl (20 mL), separated and the organic layer was extrated with water (20 mL). The combined aqueous phases were neutralized with solid sodium bicarbonate, extracted with ethyl acetate (2×25 mL), combined, dried (MgSO₄) and evaporated to a brown gum. The residue was purified on reversed phase HPLC (5 μm, 5×2.1 cm, 10-100% acetonitrile—water with 0.1% TFA, 20 mL/min., 15 min run time, 220 nm detection) to leave 17 (3×CF₃CO₂H, 2×H₂O) as a beige crystalline solid (60 mg, 48%). ESMS; M+H=578: CHN calculated for C₃₈H₄₄F₉N₅O₁₃ C 48.05, H 4.67, N 7.37; Found C 48.35, H 4.58, N 7.26.

### EXAMPLE 13

The procedure of Example 10 was repeated, except that 4-ethoxy-3-hydroxybenzaldehyde was substituted for 3′-hydroxy-4′methoxyphenylacetic acid in step (g). The 4-ethoxy-3-hydroxybenzaldehyd was synthesized as described in (a) and reacted with compound 7A as described in (b).

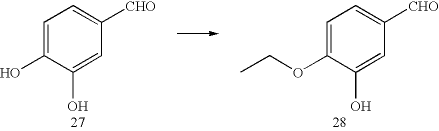

(a) Synthesis of 4-ethoxy-3-hydroxybenzaldehyde (28):

To a mixture of sodium hydride (60% mineral oil suspension, 120 mg, 3.0 mMol) and DMF (10 mL) in a dry, 20 mL scintillation vial under nitrogen atmosphere was added 3,4-dihydroxybenzaldehyde (414 mg, 3.0 mMol). The reaction mixture was shaken on an orbital shaker for 1 h then iodoethane (1.4 g, 9.0 mMol, 0.70 mL) was added. Shaking continued for 16 h then the contents were dissolved in ethyl acetate (50 mL) and water (50 mL). The organic layer was further washed with water (2×50 mL) and extracted with 1N NaOH (2×30 mL). The combined basic extracts were neutralized with conc. HCl (pH 1) and extracted with ethyl acetate (2×40 mL), dried (MgSO₄) and evaporated. The residue was chromatographed on silica gel (eluted with 1:1 hexane—ethyl acetate) to leave 28 as a brown gum (0.10 g, 20%). ¹H-NMR (CDCl₃); δ 9.86 (s, 1H), 7.45 (s, 1H), 7.41 (d, 1H, J=9.0 Hz), 6.95 (d, 1H, J=8.1 Hz), 5.78 (bs, 1H), 4.22 (q, 2H, J=7.0 Hz), 1.51 (t, 3H, J=7.0 Hz): ESMS LC/MS indicated >99% purity, M−H=165.

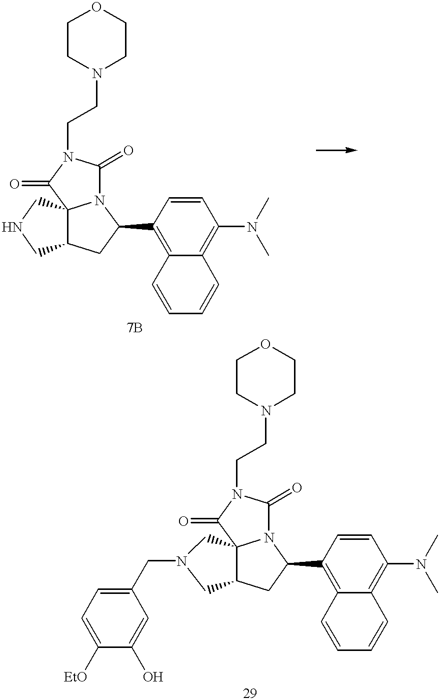

(b) Addition of the Benzyl Moiety to Form (trans)-5-[4-(dimethylamino)-1-naphthyl]-8-(4-ethoxy-3-hydroxybenzyl)-2-(2-morpholin-4-ylethyl)hexahydro-1H-pyrrolo[3′,4′:2,3]pyrrolo[1,2-c]imidazole-1,3(2H)-dione (29):

A solution of pyrrolidine 7B (0.12 g, 0.25 mMol) in DCM (1.8 mL) and acetic acid (0.20 mL) in an 8-mL scintillation vial was treated with aldehyde 28 (62 mg, 0.37 mMol) and sodium triacetoxyborohydride (80 mg, 0.37 mMol). The reaction mixture was placed on an orbital shaker, agitated for 1 h, diluted with ethyl acetate (10 mL) and water (10 mL) and the organic layer was separated, dried (MgSO₄) and evaporated. The residue was purified by semi-prep reversed phase HPLC. The dried fractions containing product were treated with 1M sodium carbonate (10 mL) and extracted with ethyl acetate (10 mL). The organic layer was dried (MgSO₄) and evaporated to leave 29 as a powdery solid (31 mg, 20%). ¹H-NMR (CDCl₃); δ 8.36 (d, 1H, J=8.3 Hz), 8.29 (d, 1H, J=8.3 Hz), 7.62 (t, 1H, J=6.7 Hz), 7.53 (t, 1H, J=7.9 Hz ), 7.28 (s, 1H), 7.00 (d, 1H, J=8.3 Hz), 6.99 (s, 1H), 6.83 (d, 1H, J=8.3 Hz), 6.78 (d, 1H, J=7.9 Hz), 5.77 (dd, 1H, J=12.3 Hz, J=4.0 Hz), 5.68 (s, 1H), 4.11 (q, 2H, J=7.1 Hz), 3.59 (ABq, 2H, J=12.7 Hz), 3.58 (bs, 4H), 3.43 (m, 2H), 3.28 (d, 1H, J=9.9 Hz), 3.13 (t, 1H J=7.9 Hz), 2.93 (dd, 1H, J=23.4 Hz, J=9.5 Hz), 2.91 (s, 6H), 2.77 (t, 1H, J=7.9 Hz), 2.62 (m, 1H), 2.39 (m, 7H ), 2.15 (dd, 1H, J=12.3 Hz, J=4.4 Hz), 1.45 (t, 3H, J=7.1 Hz): ¹³C-NMR (CDCl₃); δ 174.65, 157.41, 151.46, 145.91, 145.05, 134.39, 128.80, 126.53, 125.63, 125.03, 124.83, 124.40, 123.87, 119.83, 114.62, 112.67, 111.48, 77.24, 67.14, 64.62, 63.01, 61.18, 61.10, 58.90, 55.01, 53.36, 45.24, 44.87, 38.08, 35.71, 29.71, 14.94: ESMS; M+H=615: HPLC indicated >93% purity.

### EXAMPLE 14

The procedure of Example 10 was repeated, except that 3-hydroxy-4-n-propoxy-benzaldehyde was substituted for 3′-hydroxy-4′methoxyphenylacetic acid in step (g). The 4-ethoxy-3-hydroxybenzaldehyd was synthesized as described in (a) and reacted with compound 7A as described in (b).

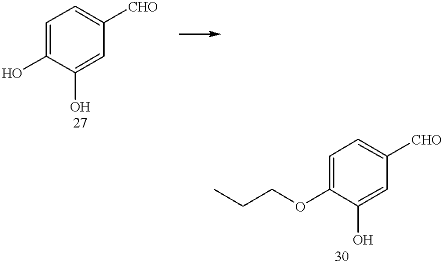

(a) Formation of 3-hydroxy-4-n-propoxy-benzaldehyde (30):

To a mixture of sodium hydride (60% mineral oil suspension, 120 mg, 3.0 mMol) and DMF (10 mL) in a dry, 20 mL scintillation vial under nitrogen atmosphere was added 3,4-dihydroxybenzaldehyde (414 mg, 3.0 mMol). The reaction mixture was shaken on an orbital shaker for 1 h then iodoproane (1.5 g, 9.0 mMol, 0.88 mL) was added. Shaking continued for 16 h then the contents were dissolved in ethyl acetate (50 mL) and water (50 mL). The organic layer was further washed with water (2×50 mL) and extracted with 1N NaOH (2×30 mL). The combined basic extracts were neutralized with conc. HCl (pH=1) and extracted with ethyl acetate (2×40 mL), dried (MgSO₄) and evaporated. The residue was chromatographed on silica gel (eluted with 1:1 hexane—ethyl acetate) to leave 30 as a brown gum (60 mg, 11%). ¹H-NMR (CDCl₃); δ 9.78 (s, 1H), 7.43 (m, 2H), 6.97 (d, 1H, J=7.8 Hz), 5.76 (s, 1H), 4.11 (t, 2H, J=6.6 Hz), 1.91 (m, 2H), 1.09 (t, 3H, J=7.6 Hz): LC/ESMS indicated purity >99%, M−H=179.

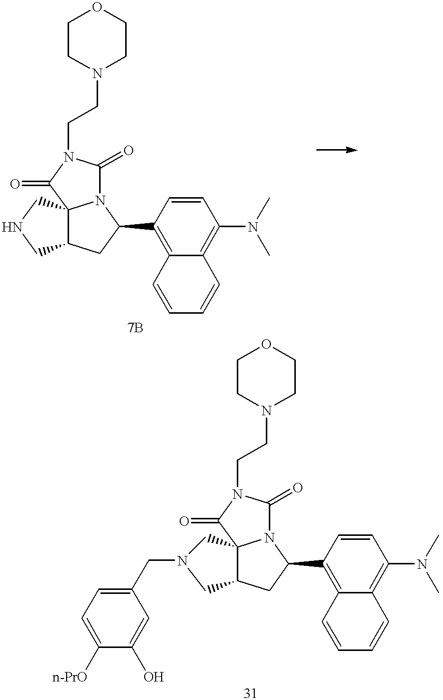

(b) Addition of the Benzyl Moiety to Form (trans)-5-[4-(dimethylamino)-1-naphthyl]-8-(3-hydroxy-4-propoxybenzyl)-2-(2-morpholin-4-ylethyl)hexahydro-1H-pyrrolo[3′,4′:2,3]pyrrolo[1,2-c]imidazole-1,3(2H)-dione (31):

A solution of pyrrolidine 7B (0.12 g, 0.25 mMol) in DCM (1.8 mL) and acetic acid (0.20 mL) in an 8-mL scintillation vial was treated with aldehyde 30 (68 mg, 0.37 mMol) and sodium triacetoxyborohydride (80 mg, 0.37 mMol). The reaction mixture was placed on an orbital shaker, agitated for 1 h, diluted with ethyl acetate (10 mL) and water (10 mL) and the organic layer was separated, dried (MgSO₄) and evaporated. The residue was purified by semi-prep reversed phase HPLC. The dried fractions containing product were treated with 1 M sodium carbonate (10 mL) and extracted with ethyl acetate (10 mL). The organic layer was dried (MgSO₄) and evaporated to leave 31 as a powdery solid (70 mg, 45%). ¹H-NMR (CDCl₃); δ 8.60 (d, 1H, J=8.7 Hz), 8.29 (d, 1H, J=8.3 Hz), 7.63 (t, 1H, J=8.3 Hz), 7.53 (t, 1H, J=7.5 Hz), 7.27 (d, 1H, J=7.9 Hz), 7.00 (d, 1H, J=7.5 Hz), 6.99 (s, 1H), 6.83 (d, 1H, J=8.3 Hz), 6.78 (d, 1H, J=8.3 Hz), 5.77 (dd, 1H, J=12.3 Hz, J=4.0 Hz), 5.67 (s, 1H), 4.00 (t, 2H, J=6.3 Hz), 3.65 (d, 1H, J=12.7 Hz), 3.58 (bt, 4H, J=4.4 Hz), 3.54 (d, 1H, J=12.7 Hz), 3.42 (m, 2H), 3.28 (d, 1H, J=9.9 Hz), 3.13 (dd, 1H, J=7.6 Hz, J=7.6 Hz), 2.95 (d, 1H, J=9.5 Hz), 2.91 (s, 6H), 2.77 (t, 1H, J=7.7 Hz), 2.61 (m, 1H), 2.39 (m, 7H), 2.15 (dd, 1H, J=12.3 Hz, J=4.4 Hz), 1.84 (m, 2H), 1.05 (t, 3H, J=7.5 Hz): ¹³C-NMR (CDCl₃); δ 174.64, 157.40, 151.46, 145.93, 145.17, 134.39, 131.84, 128.79, 126.53, 125.63, 125.03, 124.83, 124.40, 123.87, 119.83, 114.60, 112.67, 111.49, 77.25, 70.58, 67.12, 63.00, 61.17, 61.10, 58.90, 55.00, 53.35, 45.23, 44.87, 38.08, 35.69, 22.62, 10.50: HPLC indicated >93% purity: ESMS; M+H=628.

### EXAMPLE 15

The procedure of Example 10 was repeated, except that 4-butoxy-3-hydroxybenzaldehyde was substituted for 3′-hydroxy-4′methoxyphenylacetic acid in step (g).

The 4-ethoxy-3-hydroxybenzaldehyd was synthesized as described in (a) and reacted with compound 7A as described in (b).

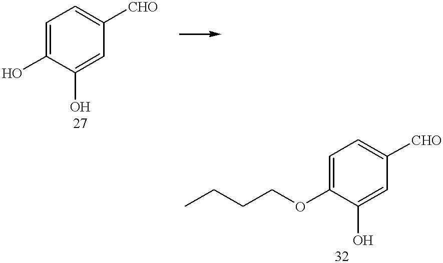

(a) Synthesis of 4-n-butyloxy-3-hydroxybenzaldehyde (32):

To a mixture of sodium hydride (60% mineral oil suspension, 120 mg, 3.0 mMol) and DMF (10 mL) in a dry, 20 mL scintillation vial under nitrogen atmosphere was added 3,4-dihydroxybenzaldehyde (414 mg, 3.0 mMol). The reaction mixture was shaken on an orbital shaker for 1 h then iodobutane (1.7 g, 9.0 mMol, 1.0 mL) was added. Shaking continued for 16 h then the contents were dissolved in ethyl acetate (50 mL) and water (50 mL). The organic layer was further washed with water (2×50 mL) and extracted with 1N NaOH (2×30 mL). The combined basic extracts were neutralized with conc. HCl (pH=1) and extracted with ethyl acetate (2×40 mL), dried (MgSO₄) and evaporated. The residue was chromatographed on silica gel (eluted with 1:1 hexane—ethyl acetate) to leave 32 as a brown gum (0.22 g, 38%). ¹H-NMR (CDCl₃); δ 9.85 (s, 1H), 7.43 (m, 2H), 6.97 (d, 1H, J=8.2 Hz), 5.74 (s, 1H), 4.15 (t, 1H, J=6.6 Hz), 1.85 (m, 2H), 1.52 (m, 2H), 1.02 (t, 3H, J=7.3 Hz): LC/ESMS indicated purity >99%, M−H=193.

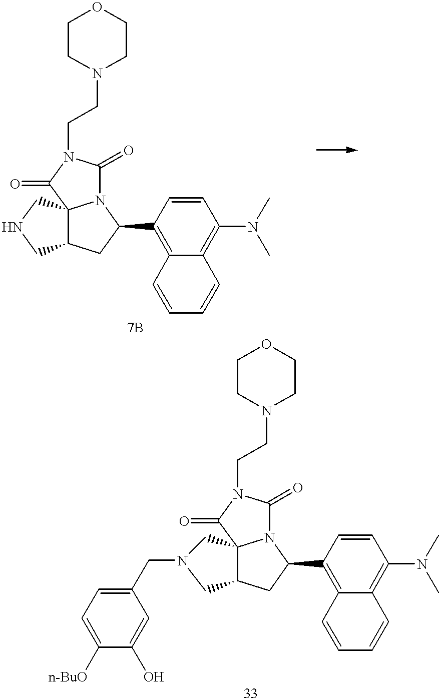

(b) Addition of the Benzyl Moiety to Form (trans)-8-(4-butoxy-3-hydroxybenzyl)-5-[4-(dimethylamino)-1-naphthyl]-2-(2-morpholin-4-ylethyl)hexahydro-1H-pyrrolo[3′,4′:2,3]pyrrolo[1,2-c]imidazole-1,3(2H)-dione (33):

A solution of pyrrolidine 7B (0.12 g, 0.25 mMol) in DCM (1.8 mL) and acetic acid (0.20 mL) in an 8-mL scintillation vial was treated with aldehyde 32 (73 mg, 0.37 mMol) and sodium triacetoxyborohydride (80 mg, 0.37 mMol). The reaction mixture was placed on an orbital shaker, agitated for 1 h, diluted with ethyl acetate (10 mL) and water (10 mL) and the organic layer was separated, dried (MgSO₄) and evaporated. The residue was purified by semi-prep reversed phase HPLC. The dried fractions containing product were treated with 1M sodium carbonate (10 mL) and extracted with ethyl acetate (10 mL). The organic layer was dried (MgSO₄) and evaporated to leave 33 as a powdery solid (90 mg, 56%). ¹H-NMR (CDCl₃); δ 8.36 (d, 1H, J=8.7 Hz), 8.29 (d, 1H, J=8.3 Hz), 7.62 (dd, 1H, J=7.1 Hz, J=7.1 Hz), 7.53 (dd, 1H, 7.5 Hz, J=7.5 Hz), 7.27 (d, 1H, J=9.9 Hz), 7.00 (d, 1H, J=7.9 Hz), 6.99 (s, 1H), 6.83 (d, 1H, J=7.5 Hz), 6.79 (d, 1H, J=7.9 Hz), 5.77 (dd, 1H, J=12.3 Hz, J=4.4 Hz), 5.65 (s, 1H), 4.04 (t, 2H, J=6.7 Hz), 3.64 (d, 1H, J=12.7 Hz), 3.59 (bs, 4H), 3.54 (d, 1H, J=13.1 Hz), 3.43 (m, 2H), 3.29 (d, 1H, J=9.9 Hz), 3.13 (t, 1H, J=7.9 Hz), 2.96 (d, 1H, J=9.1 Hz), 2.91 (s, 6H), 2.77 (t, 1H, J=8.3 Hz), 2.52 (m, 1H), 2.40 (m, 7H), 2.15 (dd, 1H, J=12.3 Hz, J=4.4 Hz), 1.81 (m, 2H), 1.51 (m, 2H), 0.99 (t, 3H, J=7.1 Hz): ): ¹³C-NMR (CDCl₃); δ 174.64, 157.40, 151.46, 145.92, 145.20, 134.40, 128.79, 126.53, 125.62, 125.03, 124.84, 124.41, 123.87, 119.84, 114.58, 112.67, 111.42, 77.25, 68.78, 67.10, 63.00, 61.17, 61.09, 58.90, 55.00, 53.33, 45.23, 44.87, 38.08, 35.66, 31.35, 29.71, 19.26, 13.83: LC/MS; >95% purity, M+H=642.

### EXAMPLE 16

The procedure of Example 10 was repeated, except that 4-methoxy-3-nitrobenzaldehyde was substituted for 3′-hydroxy-4′methoxyphenylacetic acid in step (g). The benzyl group is added in step (a) and the nitro moiety on the benzyl group converted to an amino moiety in step (b).

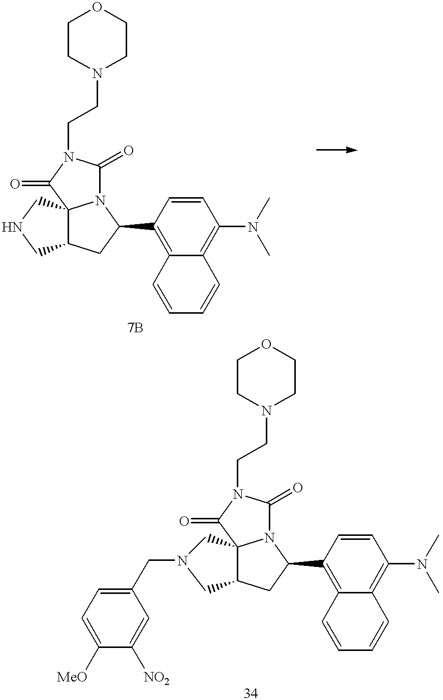

(a) Addition of the Benzyl Moiety to Form (trans)-8-(4-methoxy-3-nitro-benzyl)-5-[4-(dimethylamino)-1-naphthyl]-2-(2-morpholin-4-ylethyl)hexahydro-1H-pyrrolo[3′,4′:2,3]pyrrolo[1,2-c]imidazole-1,3(2H)-dione (34):

To a solution of pyrrolidine 7B (0.36 g, 0.77 mMol) and 4-methoxy-3-nitrobenzaldehyde (0.17 g, 0.92 mMol) in dichloromethane (5 mL) was added sodium triacetoxyborohydride (0.20 g, 0.92 mMol) and the solution stirred for 1 h. The reaction mixture was diluted with ethyl acetate (25 mL) and extracted with 1N HCl (2×25 mL). The combined aqueous extracts were washed with ethyl acetate (25 mL), neutralized with solid sodium carbonate (pH=10) and extracted with ethyl acetate (2×25 mL). The combine organic extracts were dried (MgSO₄) and evaporated to leave 34 as light yellow, foamy solid (0.33 g, 68%). ¹H-NMR (CDCl₃); δ 8.30 (d, 2H, J=8.7 Hz), 7.87 (d, 1H, J=1.6 Hz), 7.57 (m, 3H), 7.27 (d, 1H, J=7.8 Hz), 7.06 (d, 1H, J=8.6 Hz), 7.01 (d, 1H, J=7.8 Hz), 5.68 (dd, 1H, J=12.5 Hz, J=4.4 Hz), 3.97 (s, 3H), 3.3.72 (d, 1H, J=13.2 Hz), 3.65 (d, 1H J=13.2 Hz), 3.59 (t, 4H, J=4.4 Hz), 3.43 (t, 2H, J=6.4 Hz), 3.27 (d, 1H, J=10.0 Hz), 3.15 (t, 1H, J=6.2 Hz), 2.98 (d, 1H, J=10.0 Hz), 2.91 (s, 6H), 2.85 (q, 1H, J=8.0 Hz), 2.55 (m, 1H), 2.48 (m, 6H), 2.16 (dd, 1H, J=10.0 Hz, J=4.3 Hz): LC/MS; >99% purity, M+H=629.

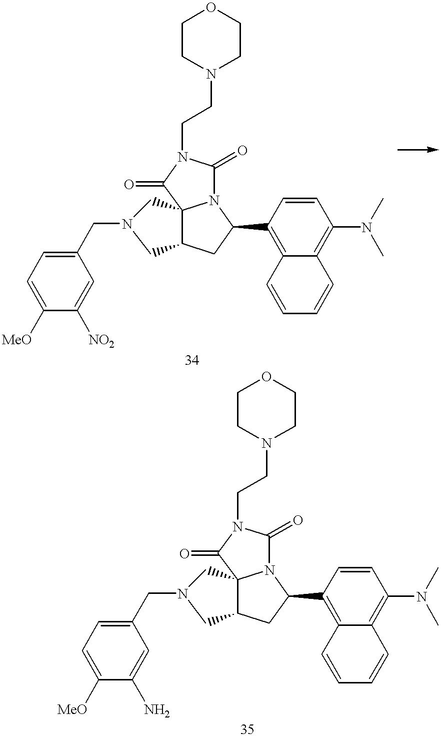

(b) Conversion of the Nitro Group to Form (trans)-8-(3-amino-4-methoxybenzyl)-5-[4-(dimethylamino)-1-naphthyl]-2-(2-morpholin-4-ylethyl)hexahydro-1H-pyrrolo[3′,4′:2,3]pyrrolo[1,2-c]imidazole-1,3(2H)-dione (35):

A mixture of nitro compound 34 (0.10 g, 0.16 mMol) and 10% Pd/C (25 mg) in ethyl acetate (2 mL) was purged with hydrogen gas and stirred 16 h under hydrogen atmosphere (balloon). The catalyst was filtered with the aid of celite, washed with methanol (3×3 mL) and evaporated. The residue was purified by semi-prep reversed phase HPLC. The fractions containing product were combined, diluted with 1M sodium carbonate (50 mL) and extracted with ethyl acetate (2×50 mL). The combined organic extracts were dried (MgSO₄) and evaporated to leave 35 as a light tan powdery solid (48 mg, 50%). ¹H-NMR (CDCl₃); d 8.37 (d, 1H, J=8.3 Hz), 8.30 (d, 1H, J=8.3 Hz), 7.59 (dd, 1H, J=7.1 Hz, J=6.8 Hz), 7.53 (dd, 1H, J=7.1 Hz, J=8.0 Hz), 7.28 (d, 1H, J=7.9 Hz), 7.01 (d, 1H, J=7.9 Hz), 6.79 (d, 1H, J=1.6 Hz), 6.73 (d, 1H, J=8.3 Hz), 6.70 (dd, 1H, J=7.9 Hz, J=1.2 Hz), 5.78 (dd, 1H, J=12.3 Hz, J=4.0 Hz), 3.85 (s, 3H), 3.80 (bs, 2H), 3.59 (m, 5H), 3.50 (d, 1H, J=13.1 Hz), 3.43 (m, 2H), 3.30 (d, 1H, J=9.9 Hz), 3.12 (t, 1H, J=7.9 Hz), 2.95 (d, 1H, J=9.5 Hz), 2.91 (s, 6H), 2.90 (d, 1H, J=9.5 Hz), 2.76 (t, 1H, J=8.7 Hz), 2.62 (m, 1H), 2.40 (m, 7H), 2.16 (dd, 1H, J -12.3 Hz, J=4.4 Hz): ¹³C-NMR (CDCl₃); d 174.65, 157.42, 151.46, 146.60, 136.28, 134.42, 131.60, 128.85, 126.42, 125.73, 125.02, 124.93, 124.40, 123.90, 118.23, 114.89, 112.78, 110.22, 67.13, 62.99, 61.26, 61.14, 59.01, 55.60, 55.03, 53.36, 45.24, 44.93, 38.06, 35.69, 29.71: HPLC indicated >97% purity: ESMS; M+H=599.

### EXAMPLE 17

The procedure of Example 16 was repeated, except that nitro group was modified to form an acteamide as described below.

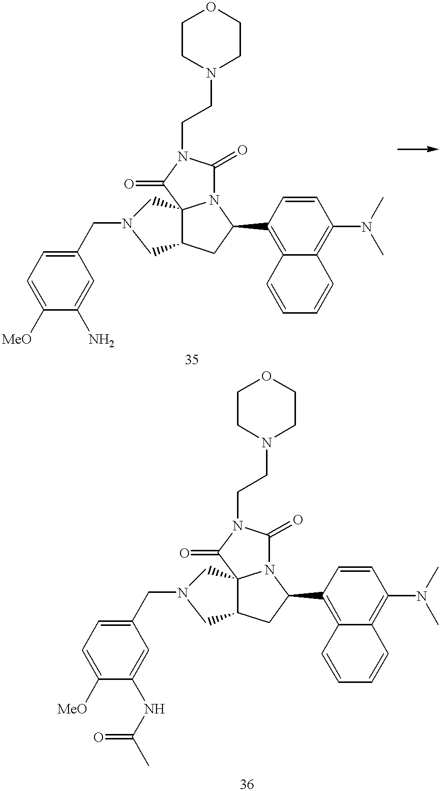

Conversion of the Nitro Group to Form N-{5-[((trans)-5-[4-(dimethylamino)-1-naphthyl]-2-(2-morpholin-4-ylethyl)-1,3-dioxohexahydro-1H-pyrrolo[3′,4′:2,3]pyrrolo[1,2-c]imidazol-8(9H)-yl)methyl]-2-methoxyphenyl}acetamide (36):

To a solution of the aniline 35 (36 mg, 60 μMol) in dichloromethane (2 mL) was added resin bound N-methylmorpholine (1% DVB-PS, 1.9 mMol/g, 0.16 g, 0.30 mMol) and the mixture was agitated on an orbital shaker for 5 mins. Acetic anhydride (9.2 mg, 90 μMol, 8.3 μL) was added and the mixture shook an additional 48 h. Aminomethyl resin (1% DVB-PS, 2.4 mMol/g, 50 mg, 0.12 mMol) was added, the reaction mixture was shaken 2 h, filtered, washed with dichloromethane (2×2 mL) and the filtrate was evaporated. The residue was purified by semi-prep reversed phase HPLC, the fractions containing product were combined, neutralized with saturated aqueous sodium bicarbonate, extracted with ethyl acetate (2×15 mL), combined dried (MgSO₄) and evaporated to leave 36 as a light tan powder (24 mg, 63%). ¹H-NMR (CDCl₃); d 8.33 (s, 1H), 8.32 (d, 1H, J=9.0 Hz), 8.29 (d, 1H, J=8.3 Hz), 7.73 (s, 1H), 7.53 (m, 2H), 7.27 (d, 1H, J=9.0 Hz), 7.12 (d, 1H, J=8.3 Hz), 7.00 (d, 1H, J=7.6 Hz), 6.84 (d, 1H, J=8.3 Hz), 5.72 (dd, 1H, J=12.5 Hz, J=4.5 Hz), 5.30 (s, 1H), 3.88 (s, 3H), 3.67 (m, 2H), 3.59 (bs, 4H), 3.42 (m, 2H), 3.26 (d, 1H, J=10.0 Hz), 3.13 (t, 1H, J=7.6 Hz), 2.96 (m, 2H), 2.91 (s, 6H), 2.81 (t, 1H, J=8.8 Hz), 2.62 (q, 1H, J=9.4 Hz), 2.41 (m, 6H), 2.17 (s, 3H), 2.14 (d, 1H, J=4.5 Hz): ESMS; M+H=641: HPLC; >97% purity.

### EXAMPLE 18

The procedure of Example 16 was repeated, except that nitro group was modified to form a trifluoroacteamide as described below.

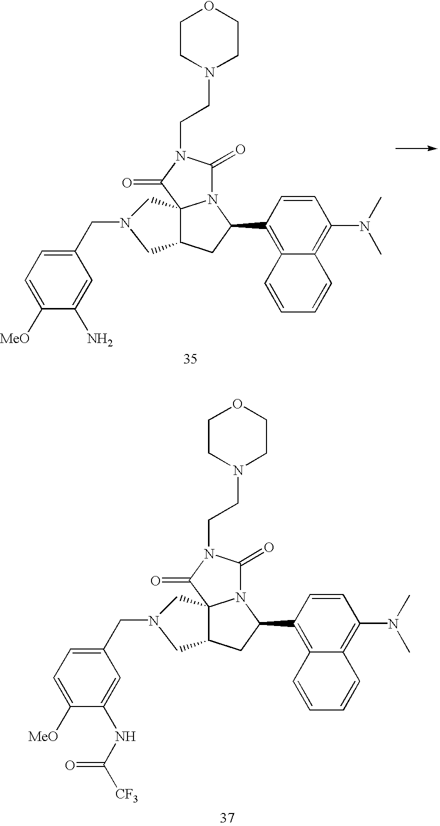

Conversion of the Nitro Group to Form N-{5-[((trans)-5-[4-(dimethylamino)-1-naphthyl]-2-(2-morpholin-4-ylethyl)-1,3-dioxohexahydro-1H-pyrrolo[3′,4′:2,3]pyrrolo[1,2-c]imidazol-8(9H)-yl)methyl]-2-methoxyphenyl}-2,2,2-trifluoroacetamide (37):

To a solution of the aniline 35 (36 mg, 60 μMol) in dichloromethane (2 mL) was added resin bound N-methylmorpholine (1% DVB-PS, 1.9 mMol/g, 0.16 g, 0.30 mMol) and the mixture was agitated on an orbital shaker for 5 mins. Trifluoroacetic anhydride (38 mg, 180 μMol, 25 μL) was added and the mixture shook an additional 48 h. Aminomethyl resin (1% DVB-PS, 2.4 mMol/g, 50 mg, 0.12 mMol) was added, the reaction mixture was shaken 2 h, filtered, washed with dichloromethane (2×2 mL) and the filtrate was evaporated. The residue was purified by semi-prep reversed phase HPLC, the fractions containing product were combined, neutralized with saturated aqueous sodium bicarbonate, extracted with ethyl acetate (2×15 mL), combined dried (MgSO₄) and evaporated to leave 37 as a light tan powder (23 mg, 55%). ¹H-NMR (CDCl₃); δ 8.55 (s, 1H), 8.29 (m, 3H), 7.52 (m, 2H), 7.28 (d, 1H, J=7.7 Hz), 7.24 (d, 1H, J=7.9 Hz), 7.00 (d, 1H, J=8.0 Hz), 6.90 (d, 1H, J=8.4 Hz), 5.71 (dd, 1H, J=12.0 Hz, J=4.4 Hz), 5.30 (s, 1H), 3.93 (s, 3H), 3.67 (dd, 2H, J=19.0 Hz, J=12.8 Hz), 3.59 (bs, 4H), 3.44 (m, 2H), 3.26 (d, 1H, J=9.8 Hz), 3.15 (t, 1H, J=7.7 Hz), 2.98 (d, 1H, J=10.2 Hz), 2.94 (d, 1H, J=8.8 Hz), 2.91 (s, 6H), 2.82 (dd, 1H, J=8.8 Hz, J=8.4 Hz), 2.64 (dd, 1H, J=14.0 Hz, J=6.8 Hz), 2.41 (bs, 6H), 2.15 (dd, 1H, J=12.4 Hz, J=4.4 Hz): ¹³C-NMR (CDCl₃); δ 174.62, 157.32,154.34 (q, J=37.3), 151.47, 147.56, 134.36, 131.63, 128.80, 126.32, 125.92, 125.57, 124.97, 124.87, 124.33, 123.99, 120.59, 115.71 (q, J=288.5), 112.71, 110.30, 67.03, 62.99, 61.07, 61.00, 58.72, 56.08, 54.97, 53.41, 53.31, 45.22, 44.83, 38.06, 35.62, 29.71: ESMS; M+H=695: HPLC purity=95%.

### EXAMPLE 19

The procedure of Example 16 was repeated, except that nitro group was modified to form a sulfonylamide as described below.

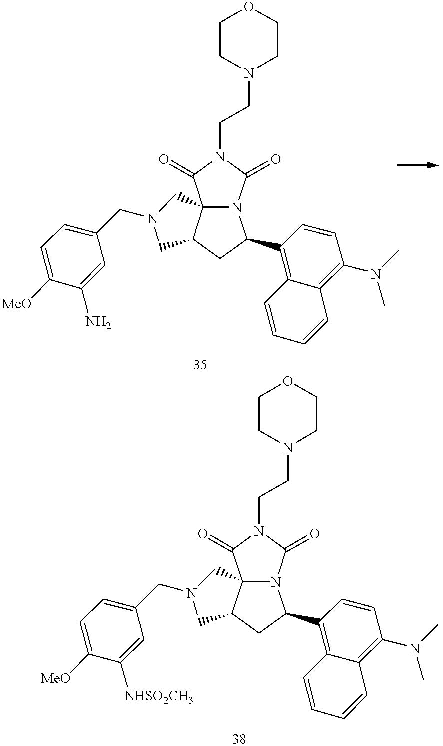

Conversion of the Nitro Group to Form N-{5-[((trans)-5-[4-(dimethylamino)-1-naphthyl]-2-(2-morpholin-4-ylethyl)-1,3-dioxohexahydro-1H-pyrrolo[3′,4′:2,3]pyrrolo[1,2-c]imidazol-8(9H)-yl)methyl]-2-methoxyphenyl}methanesulfonamide (38):

To a solution of the aniline 35 (36 mg, 60 μMol) in dichloromethane (2 mL) was added resin bound N-methylmorpholine (1% DVB-PS, 1.9 mMol/g, 0.16 g, 0.30 mMol) and the mixture was agitated on an orbital shaker for 5 mins. Methanesulfonyl chloride (45 mg, 0.39 mMol, 30 μL) was added and the mixture shook an additional 48 h. Aminomethyl resin (1% DVB-PS, 2.4 mMol/g, 150 mg, 0.36 mMol) was added, the reaction mixture was shaken 2 h, filtered, washed with dichloromethane (2×2 mL) and the filtrate was evaporated. The residue was purified by semi-prep reversed phase HPLC, the fractions containing product were combined, neutralized with saturated aqueous sodium bicarbonate, extracted with ethyl acetate (2×15 mL), combined, dried (MgSO₄) and evaporated to leave 38 as a light tan powder (11 mg, 27%). ¹H-NMR (CDCl₃); δ 8.32 (d, 1H, J=8.0 Hz), 8.29 (d, 1H, J=8.7 Hz), 7.58 (dd, 1H, J=6.6 Hz, J=6.6 Hz), 7.52 (dd, 1H, J=8.4 Hz, J=8.4 Hz), 7.49 (s, 1H), 7.28 (d, 1H, J=7.7 Hz), 7.18, (d, 1H, J=8.4 Hz), 7.00 (d, 1H, J=7.6 Hz), 6.87 (d, 1H, J=8.4 Hz), 6.77 (s, 1H), 5.70 (dd, 1H, J=12.2 Hz, J=4.2 Hz), 5.30 (s, 1H), 3.88 (s, 6H), 3.70 (d, 1H, J=13.3), 3.61 (m, 5H), 3.45 (t, 2H, J=6.3 Hz), 3.28 (d, 1H, J=10.1 Hz), 3.15 (dd, 1H, J=7.3 Hz, J=5.0 Hz), 3.00 (d, 1H, J=9.8 Hz), 2.94 (d, 1H, J=10.1 Hz), 2.91 (s, 6H), 2.84 (s, 3H), 2.79 (t, 1H, J=8.4 Hz), 2.64 (dd, 1H, J=12.8 Hz, J=6.2 Hz), 2.42 (bs, 6H), 2.16 (dd, 1H, J=12.2 Hz, J=4.2 Hz): ESMS; M+H=677: HPLC purity=91%.

### EXAMPLE 20

The procedure of Example 16 was repeated, except that nitro group was modified to form butanamide as described below.

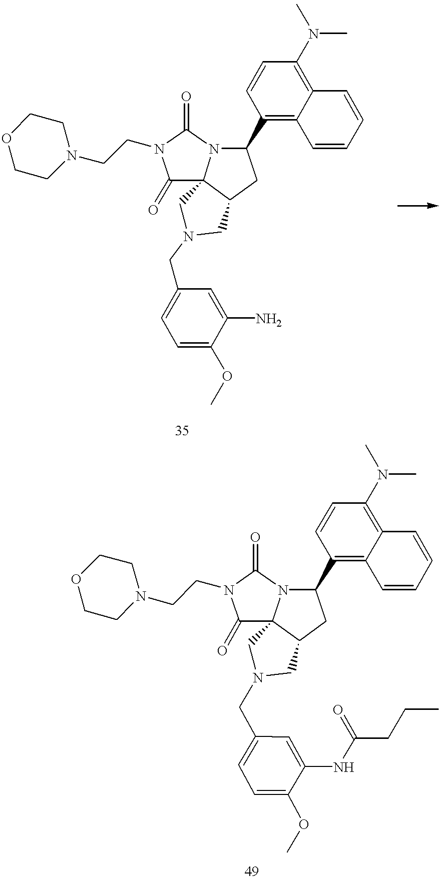

Conversion of the Nitro Group to Form N-{5-[((trans)-5-[4-(dimethylamino)-1-naphthyl]-2-(2-morpholin-4-ylethyl)-1,3-dioxohexahydro-1H-pyrrolo[3′,4′:2,3]pyrrolo [1,2-c]imidazol-8(9H)-yl)methyl]-2-methoxyphenyl}butanamide (49): In a 4 mL vial under nitrogen was placed 35 (0.050 g, 0.084 mmol) and dry dichloromethane (2 mL). To this was added N-Methyl morpholine resin (239 mg, 1.75 g/mmol) followed by butyric anhydride (20 μL, 0.125 mmol) and the mixture shaken overnight. To the mixture was added AM resin (70 mg, 2.4 g/mmol) and the vial shaken for three hours. The mixture was filtered and the solvent evaporated. The crude material was purified by RP-HPLC to yield 25 mg of 49 as a white solid (45%). ¹H NMR (CDCl₃): δ 8.28 (d, 1H, J=2 Hz), 8.30 (m, 1H), 8.24 (m, 1H), 7.79 (s, 1H), 7.63 (m, 2H), 7.36 (d, 1H, J=8 Hz), 7.27 (m, 1H), 6.96 (d, 1H, J=8 Hz), 5.48 (d, 1H, J=8 Hz), 4.40 (d, 1H, J=13 Hz), 4.27 (d, 1H, J=13 Hz), 3.92 (s, 3H), 3.87 (m, 4H), 3.68 (m, 4H), 3.57 (m, 4H), 3.19 (m, 2H), 3.14 (s, 6H), 3.85 (m, 2H), 2.74 (m, 2H), 2.37 (t, 2H, J=7 Hz), 2.21 (dd, 1H, J=5 Hz, 8 Hz), 1.73 (q, 2H, J=8 Hz), 1.00 (t, 3H, J=8 Hz): MS ES-POS=669 [M+H]: CHN for C38H48N6O5*3TFA; calc C, 52.28, H, 5.09, N, 8.31; found C, 50.86, H, 5.01, N, 8.31.

### EXAMPLE 21

The procedure of Example 16 was repeated, except that nitro group was modified to form propanamide as described below.

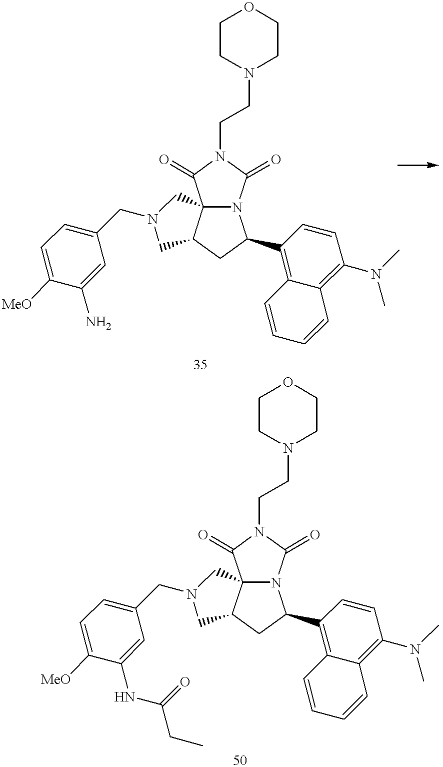

Conversion of the Nitro Group to Form N-{5-[((trans)-5-[4-(dimethylamino)-1-naphthyl]-2-(2-morpholin-4-ylethyl)-1,3-dioxohexahydro-1H-pyrrolo[3′,4′:2,3]pyrrolo[1,2-c]imidazol-8(9H)-yl)methyl]-2-methoxyphenyl}propanamide (50):

In a 4 mL vial under nitrogen was placed 35 (0.050 g, 0.084 mmol) and dry dichloromethane (2 mL). To this was added N-Methyl morpholine resin (239 mg, 1.75 g/mmol) followed by propionic anhydride (16 μL, 0.125 mmol) and the mixture shaken overnight. To the mixture was added AM resin (70 mg, 2.4 g/mmol) and the vial shaken for three hours. The mixture was filtered and the solvent evaporated. The crude material was purified by RP-HPLC to yield 25 mg of 50 as a white solid (44%). ¹H NMR (CDCl₃): δ 8.28 (d, 1H, J=2 Hz), 8.30 (m, 1H), 8.24 (m, 1H), 7.79 (s, 1H), 7.63 (m, 2H), 7.36 (d, 1H, J=8 Hz), 7.27 (m, 1H), 6.96 (d, 1H, J=8 Hz), 5.48 (d, 1H, J=8 Hz), 4.40 (d, 1H, J=13 Hz), 4.27 (d, 1H, J=13 Hz), 3.92 (s, 3H), 3.87 (m, 4H), 3.68 (m, 4H), 3.57 (m, 4H), 3.19 (m, 2H), 3.14 (s, 6H), 3.85 (m, 2H), 2.74 (m, 2H), 2.42 (q, 2H, J=8 Hz), 2.24 (m, 1H), 1.24 (m, 3H): MS ES-POS=655 [M+H]: CHN for C₃₇H_{4 6}N₆O₅*3 TFA; calc C, 51.81, H, 4.95, N, 8.43; found C, 50.49, H, 4.88, N, 8.02.

### EXAMPLE 22

The procedure of Example 10 was repeated, except that 3,4-dibenzyloxy benzaldehyde was substituted for 3′-hydroxy-4′methoxyphenylacetic acid in step (g). The synthesis of 3,4-dibenzyloxy benzaldehyde and the addition of the compound to 7A is described in (a) and removal of the benzyl groups from the benzyloxy moieties is described in (b).

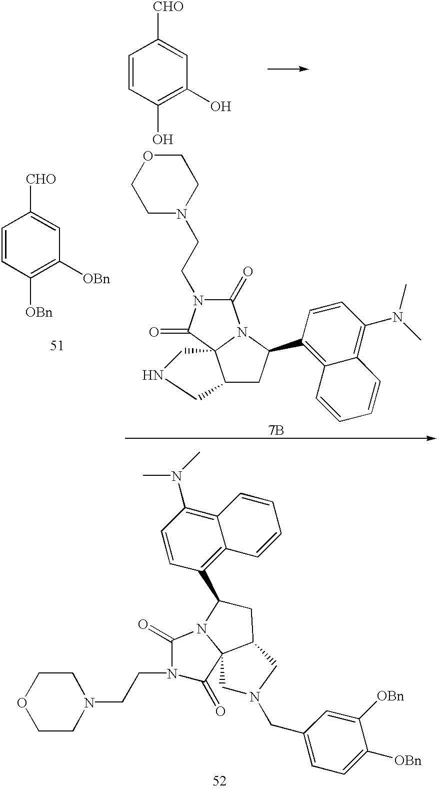

(a) Synthesis of 3,4-dibenzyloxy Benzaldehyde and Reaction with 7A to Form (trans)-8-[3,4-bis(benzyloxy)benzyl]-5-[4-(dimethylamino)-1-naphthyl]-2-(2-morpholin-4-ylethyl)hexahydro-1H-pyrrolo [3′,4′:2,3]pyrrolo[1,2-c]imidazole-1,3 (2H)-dione (52):

In a round bottom flask under nitrogen was placed 3,4-Dihydroxybenzaldehyde (l.Og, 7.24 mmol) and acetone added (22 mL). To this solution was added potassium carbonate (2.1 g, 15.2 mmol) followed by benzyl bromide (1.72 mL, 14.48 mmol) and the mixture heated to reflux for sixteen hours and partitioned between ethyl acetate and water. Washed organics with brine, dried, and concentrated to yield 2.41 g of 3,4-dibenzyloxy benzaldehyde 51 as a brown solid (104%). This material (0.16 g, 0.496 mmol) was then added as is to a solution of 7B (0.12 g, 0.248 mmol) in acetonitrile (20 mL) at room temperature. Sodium triacetoxyborohydride was added (0.105 g, 0.496 mmol) and the solution stirred for sixteen hours. The reaction was quenched with 100 mL of saturated sodium bicarbonate solution and extracted with ethyl acetate. The organic layer was washed with brine, dried, and concentrated to a small volume. Purification by flash column chromatography on silica gel using 3% methanol in dichloromethane yielded 52 as a white foamy solid (47%). ¹H NMR (CDCl₃): δ 8.32 (d, 1H, J=8 Hz), 8.29 (d, 1H, J=8 Hz), 7.49 (m, 4H), 7.36 (m, 2H), 7.32-7.1.7 (m, 7H), 7.02 (m, 2H), 6.88 (m, 2H), 5.70 (dd, 1H, J=12 Hz, 4 Hz), 5.16 (s, 2H), 5.05 (q, 2H, J=4 Hz), 3.58 (br s, 4H), 3.44 (br s, 2H), 3.25 (d, 1H, J=10 Hz), 3.11 (m, 1H), 2.91 (s, 6H), 2.87 (d, 1H, J=9 Hz), 2.71 (t, 1H, J=8 Hz), 2.61 (m, 1H), 2.39 (br s, 5H), 2.08 (dd, 1H, J=12 Hz, 8 Hz), 1.58 (br s, 4H): MS ES-POS=766.8 [M+H]: Analytical RP-HPLC shows >93% purity@220,254 nm.

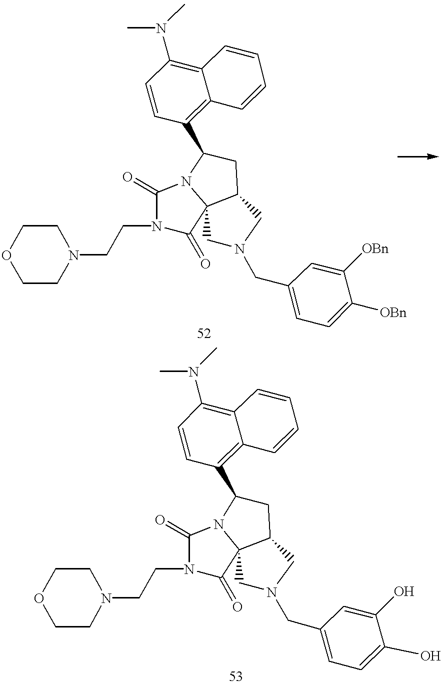

(b) Removal of the Benzyl Groups to Form (trans)-8-(3,4-dihydroxybenzyl)-5-[4-(dimethylamino)-1-naphthyl]-2-(2-morpholin-4-ylethyl) hexa-hydro-1H-pyrrolo[3′,4′:2,3]pyrrolo[1,2-c]imidazole-1,3(2H)-dione (53):

In a round bottom flask was placed 52 (0.065 g, 0.085 mmol) and THF (30 mL). The flask was purged with nitrogen and palladium on carbon (5%) was added (0.036 g, 0.017 mmol). A hydrogen balloon was attached, the flask evacuated, and a hydrogen atmosphere established. The black solution was allowed to stir for sixteen hours and was then purged with nitrogen and filtered through Celite. The solids were washed with THF and the solution concentrated to dryness on a rotovap. Purification by RP-HPLC yielded 18 mg of 53 as a white solid (36%). ¹H NMR (CDCl₃): δ 8.27 (d, 1H, J=5 Hz), 8.25 (d, 1H, J=5 Hz), 7.57 (t, 1H, J=7 Hz), 7.51 (t, 1H, J=7 Hz), 7.30 (d, 1H, J=8 Hz), 7.00 (d, 1H, J=8 Hz), 6.98 (br s, 1H), 6.85 (d, 1H, J=8 Hz), 6.81 (d, 1H, J=8 Hz)5.62 (dd, 1H, J=8 Hz, 4 Hz), 4.02 (br s, 2H), 3.79 (s, 4H), 3.67 (m, 2H), 3.59 (t, 1H, J-6 Hz), 3.56 (t, 1H, J=6 Hz), 3.51 (br s, 1H), 3.4-3.2 (m, 4H), 3.1-2.85 (m, 4H), 2.91 (s, 6H), 2.70 (q, 1H, J=12 Hz), 2.18 (m, 1H): MS ES-POS=586 [M+H]: Analytical RP-HPLC shows >93% purity@220,254 nm.

### EXAMPLE 23

The procedure of Example 10 was repeated, except that 3-Benzyloxy-4-nitro-benzaldehyde was used to add the benzyl moiety in step (g). The addition of the 3-Benzyloxy-4-nitro-benzaldehyde and conversion of the nitro group to an amino group is described below.

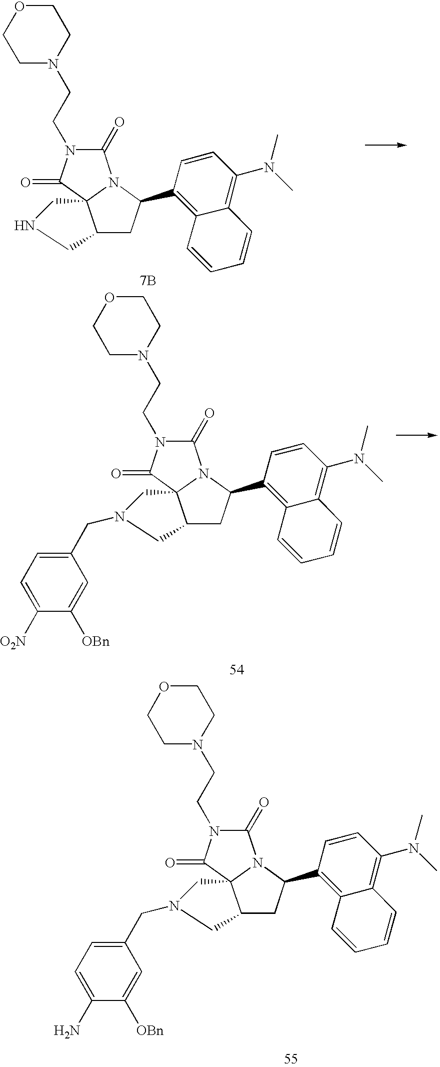

Formation of (trans)-8-(4-amino-3-benzyloxybenzyl)-5-[4-(dimethylamino)-1-naphthyl]-2-(2-morpholin-4-ylethyl)hexahydro-1H-pyrrolo[3′,4′:2,3]pyrrolo[1,2-c]imidazole-1,3(2H)-dione (55): To a solution of 7B (0.85 g, 1.31 mmol) in DMF (50 mL) was added benzenethiol, sodium salt (0.577 g, 3.93 mmol) and the solution stirred overnight. The reaction was quenched with 150 nL of 1N HCl and then extracted with ethyl acetate (2×). The aqueous layer was adjusted to pH of 8 with 6N NaOH solution and then extracted with ethyl acetate. This organic layer was dried and concentrated to a brown oil. It was redissolved in acetonitrile (60 mL) and 3-Benzyloxy-4-nitro-benzaldehyde (0.674, 2.62 mmol) added followed by sodium triacetoxyborohydride (0.555 g, 2.62 mmol) and the mixture stirred for sixteen hours. Sodium bicarbonate solution (50 mL) was added and the solution extracted with ethyl acetate. After washing with brine, drying, and concentrating to dryness the residue was purified by flash chromatography on silica using 3% methanol in dichloromethane as eluant to yield 400 mg (43% for two steps) of the intermediate 54 as a yellow solid. NMR (CDCl₃) and MS agreed with structure and material was used as is. The solid (0.40 g, 0.57 mmol) was dissolved in methanol (30 mL) and placed under a nitrogen atmosphere. Platinum oxide (0.013 g, 0.057 mmol) was added and the flask evacuated and refilled with a hydrogen atmosphere. After stirring sixteen hours the material was purged with nitrogen, filtered thru Celite, and concentrated to yield 330 mg of 55 as an off-white solid. NMR and MS agree with structure and the material was used as is.

### EXAMPLE 24

The product of Example 23 was further modified, as described below.

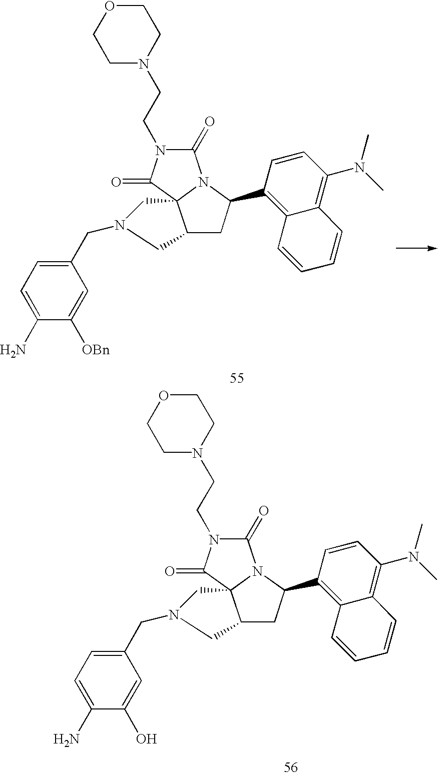

Removal of the Benzyl Group from the Benzyoxy Moiety to Form (trans)-8-(4-amino-3-hydroxybenzyl)-5-[4-(dimethylamino)-1-naphthyl]-2-(2-morpholin-4-ylethyl)hexahydro-1H-pyrrolo[3′,4′:2,3]pyrrolo[1,2-c]imidazole-1,3(2H)-dione (56):

To a solution of 55 (0.05 g, 0.074 mmol) in methanol (3 mL) was added palladium on carbon (0.05 g) and the flask purged with nitrogen. To the stirring solution was added 1,4-cyclohexadiene (0.07 mL, 0.74 mmol) and the solution stirred for sixteen hours. The solution was filtered thru Celite and concentrated to dryness. Purification by RP-HPLC yielded 19 mg of 56 as a yellow solid (44%). ¹H NMR (CDCl₃): δ 8.25 (t, 2H, J=8 Hz), 7.57 (dt, 1H, J=1 Hz, 7 Hz), 7.51 (dt, 1H, J=1 Hz, 7 Hz), 7.30 (d, 1H, J=8 Hz), 7.00 (d, 1H, J=7 Hz), 6.97 (s, 1H), 6.80 (m, 2H), 5.57 (dd, 1H, J=4 Hz, 8 Hz), 4.15-4.00 (m, 2H), 3.81 (br s, 4H), 3.71 (m, 2H), 3.61 (m, 2H), 3.39 (m, 2H), 3.18-2.96 (m, 6H), 2.91 (s, 6H), 2.70 (q, 1H, J=8 Hz), 2.18 (dd, 1H, J=4 Hz, 9 Hz): MS ES-POS=585 [M+H]: Analytical RP-HPLC >93% purity@220,254 nm.

### EXAMPLE 25

The amino moiety on the benzyl group of compound 56 was modified as described below.

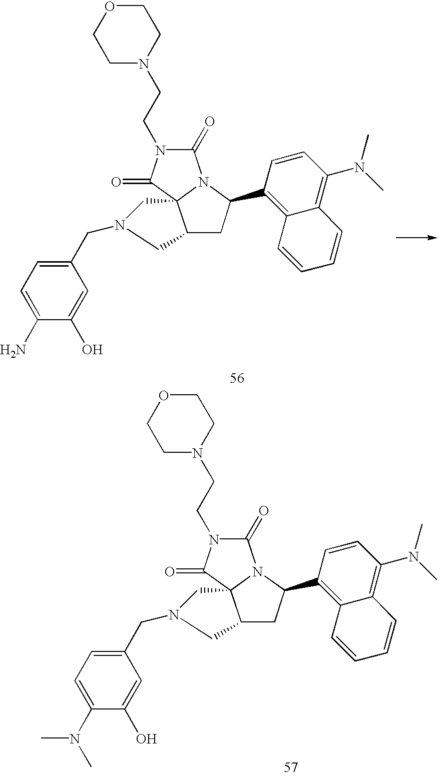

Synthesis of (trans)-8-[4-dimethylamino)-3-hydroxybenzyl]-5-[4-(dimethylamino)-1-naphthyl]-2-(2-morpholin-4-ylethyl)hexahydro-1H-pyrrolo[3′,4′:2,3]pyrrolo[1,2-c]imidazole-1,3(2H)-dione (57):

To a solution of 56 (0.040 g, 0.06 mmol) in glacial acetic acid (5 mL) was added paraformaldehyde (0.018 g, 0.59 mmol) followed by sodium cyanoborohydride (0.019 g, 0.296 mmol) and the solution stirred for sixteen hours. The solution was poured into 15 mL of 25% sodium hydroxide and extracted with dichloromethane (2×) and ethyl acetate (2×). The organics were combined, dried, and concentrated to a crude solid. MS shows parent ion. Use as is by dissolving in 6 mL of dry methanol. After purging with nitrogen palladium on carbon (0.050 g) was added followed by 1,4-cyclohexadiene (0.63 mL, 0.67 mmol) and the reaction stirred for sixteen hours. The solution was filtered thru Celite, concentrated to dryness, and purified by RP-HPLC to yield 14 mg of 57 as a white solid (34%). ¹H NMR (CDCl₃): δ 8.30 (m, 1H), 8.19 (m, 1H), 7.58 (m, 2H), 7.45-7.10 (m, 2H), 7.10 (d, 1H, J=8 Hz), 6.97 (s, 1H), 6.82 (d, 1H, J=10 Hz), 5.53 (m, 1H), 4.15 (s, 2H), 3.88 (br s, 4H), 3.80-3.15 (m, 12 H), 3.12 (s, 6H), 3.07 (m, 4H), 3.01 (s, 6H), 2.88 (d, 1H, J=7 Hz), 2.78 (m, 1H), 2.25-2.10 (m, 2H): MS ES-POS=613 [M+H]: Analytical RP-HPLC shows >80%@220,254 nm.

### EXAMPLES 26-28

The amino moiety on the benzyl group of compound 55 was modified as described below.

[chemical structure]

Modification of the Amino Group to Produce Various Compounds:

In 3×8 mL vials 55 (0.050 g, 0.074 mmol) was placed and dissolved in dichloromethane (3 mL). To this solution was added N-Methyl morpholine resin (0.37 mmol, 3.4 mmol/g) and then the following reagents added to one vial: acetic anhydride (0.023 g, 0.222 mmol), trifluoroacetic anhydride (0.047 g, 0.222 mmol), and methane sulfonyl chloride (0.025 g, 0.222 mmol). The vials were capped and shaken overnight. LC/MS showed complete removal of starting material. Aminomethyl resin (0.062 g, 0.148 mmol) was added to each reaction and the vials shaken for two hours. Each vial was filtered and concentrated to dryness. Each compound was redissolved in 3 mL of methanol and purged with nitrogen. Palladium on carbon (0.050 g, 0.47 mmol) was added to each vial followed by 1,4-cyclohexadiene (0.07 mL, 0.74 mmol) and the vials shaken for twenty four hours. Each vial was filtered and its contents concentrated to dryness. Purification of each by RP-HPLC gave the following products.

a. 27 mg of 58 as a white solid (10%). ¹H NMR (DMSO-d₆): δ 9.43 (s, 1H), 8.40 (m, 1H), 8.20 (d, 1H, J=7 Hz), 7.87 (br s, 1H), 7.56 (m, 2H), 7.35 (d, 1H, J=8 Hz), 7.06 (d, 2H, J=8 Hz), 7.03 (s, 1H), 5.57 (d, 1H, J=9 Hz), 4.6-2.9 (m, 20H), 2.85 (s, 6H), 2.67 (m, 1H), 2.21 (d, 1H, J=10 Hz), 2.10 (s, 3H): MS ES-POS=627 [M+H]: Analytical RP-HPLC >92%@220,254 nm.

b. 18 mg of 59 as a white solid (6%). ¹H NMR (DMSO-d₆): δ 10.66 (s, 1H), 8.39 (br s, 1H), 8.21 (d, 1H, J=7 Hz), 7.56 (m, 2H), 7.45 (br s, 1H), 7.36 (d, 1H, J=8 Hz), 7.12 (m, 1H), 7.06 (d, 2H, J=8 Hz), 5.57 (d, 1H, J=12 Hz), 4.6-2.9 (m, 20H), 2.85 (s, 6H), 2.68 (br s, 1H), 2.21 (m, 1H): MS ES-POS=681 [M+H]: Analytical RP-HPLC >77%@220,254 nm.

c. 4 mg of 60 as a white solid (1%). ¹H NMR (DMSO-d₆): δ 8.14 (br s, 1H), 8.11 (d, 1H, J=7 Hz), 7.57 (m, 4H), 7.28 (d, 1H, J=8 Hz), 7.25 (m, 6H), 7.05 (d, 1H, J=8 Hz), 5.61 (m, 3H), 5.08 (br s, 2H), 4.05-2.90 (m, 20H), 2.92 (s, 6H), 2.44 (s, 3H): MS ES-POS=753 [M+H]: Analytical RP-HPLC >90%@220,254 nm.

### EXAMPLE 29

The procedure of Example 10 was repeated, except that 4-quinoline carboxyaldehyde was substituted for 4-(dimethylamino)-1-naphthaldehyde in step (d). The remaining synthesis steps are detailed below

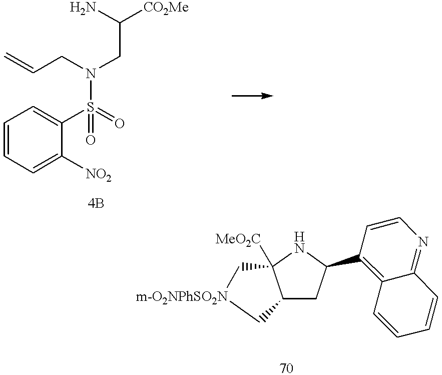

(a) Cyclization and Addition of the Naphthyl Group to Form Methyl (trans)-5-[(2- nitrophenyl)sulfonyl]-2-quinolin-3-ylhexahydropyrrolo[3,4-b]pyrrole-6a(1H)-carboxylate (70):

A solution of 4B (1.0 g, 2.9 mmol) in degassed toluene (300 mL) was treated with 4-quinoline carboxyaldehyde (732 mg, 4.7 mmol). The reaction was allowed to stir 18 h at 90° C. The solvent was removed under reduced pressure. This crude material was chromatographed in 90% ethyl acetate/hexane, and the solvent was removed under reduced pressure to yield 70 (790 mg (56%), 1.6 mmol). ¹H NMR (CDCl₃): δ 9.10 (d, J=4 Hz, 1H), 8.38 (d, J=8 Hz, 1H), 8.10-8.23 (m, 3H), 7.88 (t, J=7 Hz, 1H), 7.61-7.82 (m, 4H), 5.49 (m, 1H), 3.87 (s, 2H), 3.79 (s, 3H), 3.72 (m, 2H), 3.27 (m, 1H), 2.51 (m, 1H), 2.10 (m, 1H). MS (ESI-POS):[M+H]+483.

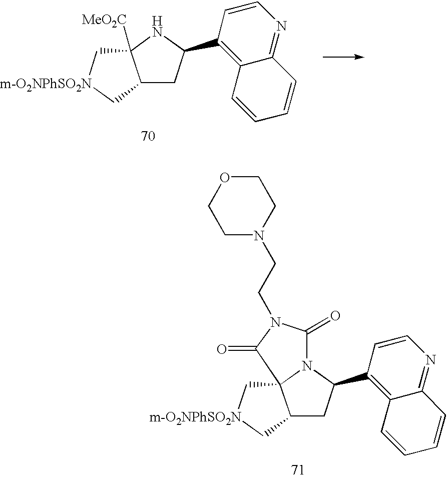

(b) Addition of the Morpholine Moiety to Form (trans)-2-(2-morpholin-4-ylethyl)-8-[(2-nitrophenyl)sulfonyl]-5-quinolin-4-ylhexahydro-1H-pyrrolo [3′,4′:2, 3]pyrrolo[1,2-c]imidazole-1,3(2H)-dione (71): A solution of 70 (700 mg, 1.5 mmol) in DCM (25 mL) and DIEA (260 mg, 2.0 mmol) was cooled in an ice bath. Phosgene (1.4 mL of 2M solution, 2.5 mmol) was added, the solution turned dark green and it was allowed to stir for 1 h at room temperature. The DCM was removed under reduced pressure, and the crude material was partitioned between ethyl acetate and 1M Na₂CO₃ solution. The organic layer was washed twice with 1M sodium carbonate solution. It was then washed with brine, dried with MgSO₄., and the solvent was removed under reduced pressure. Ethyl acetate (20 mL), DIEA (840 mg, 6.5 mmol) and N-ethyl morpholine (1.2 g, 9.2 mmol) were added, and it was allowed to stir for 3 h at 60° C. The ethyl acetate was washed with water twice, and once with brine. It was dried with MgSO₄, and the solvent was removed under reduced pressure. The crude product was chromatographed in 1% MeOH/DCM, and the solvent was removed under reduced pressure to yield 71 (250 mg (23%), 0.4 mmol). ¹H NMR (CDCl₃): δ 8.90 (d, J=4 Hz, 1H), 8.01-8.30 (m, 3H), 7.60-7.92 (m, 5H), 7.20 (d, J=4 Hz, 1H), 5.52 (m, 1H), 3.61-3.87 (m, 12H), 3.30-3.58 (m, 5H), 2.61 (m, 1H), 1.26 (m, 2H). MS (ESI-POS):[M+H]+607. Anal. Calc. for C₂₉H₃₀N₆O₇S: C, 57.42, H, 4.98, N, 13.85. Found: C, 55.27, H, 5.90, N, 14.07.

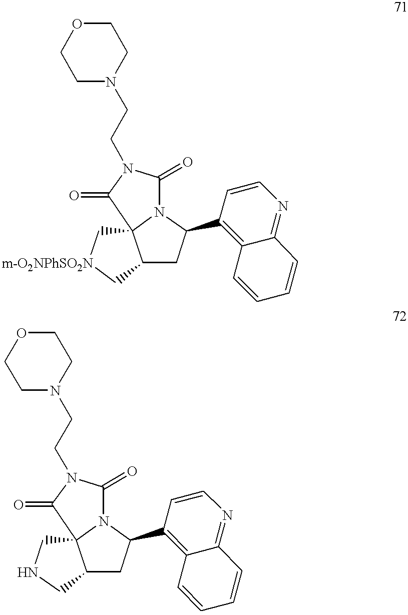

(c) Deprotection of the Amine to Form (trans)-2-(2-morpholin-4-ylethyl)-5-quinolin-4-ylhexahydro-1H-pyrrolo [3′, 4′:2,3]pyrrolo[1,2-c]imidazole-1,3(2H)-dione (72):

A solution of 71 (200 mg, 0.33 mmol) in DMF (3 mL) was treated with sodium thiophenoxide (100 mg, 0.76 mmol) and the reaction was complete after 1 h. The reaction mixture was partitioned between ethyl acetate and 3N HCl. The organic layer was washed three times with 3N HCl. The organic layer was saved. The acidic water layers were combined, neutralized with sodium carbonate and extracted three times with ethyl acetate. All of the combined ethyl acetate was washed with brine, and dried with MgSO₄. The solvent was removed under reduced pressure. This crude material was chromatographed in 8% MeOH/DCM, and the solvent was removed under reduced pressure to yield 72 (81 mg (58%),.19 mmol).

¹H NMR (CDCl₃): δ 8.92 (d, J=4 Hz, 1H), 8.32 (d, J=4 Hz, 1H), 8.17 (d, J=8 Hz, 1H), 7.75 (m, 1H), 7.64 (m, 1H), 7.24 (m, 1H), 5.30 (m, 1H), 3.61-3.87 (m, 12H), 3.30-3.58 (m, 5H), 2.63 (m, 1H), 1.27 (m, 2H). MS (ESI-POS):[M+H]+422.

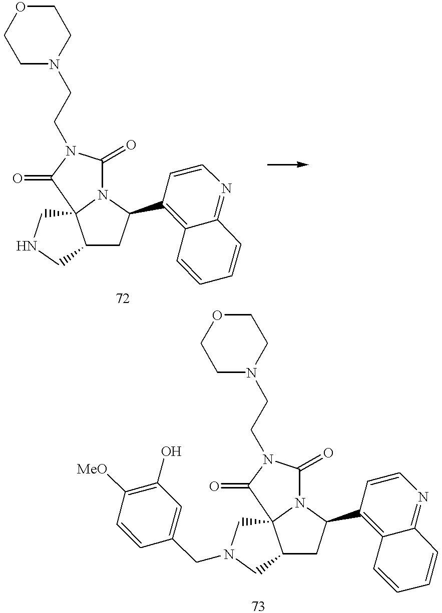

(d) Addition of the Benzyl Moiety to Provide (trans)-8-(3-hydroxy-4-methoxybenzyl)-2-(2-morpholin-4-ylethyl)-5-quinolin-4-ylhexahydro-1H-pyrrolo [3′, 4′:2,3]pyrrolo[1,2-c]imidazole-1,3(2H)-dione (73):

A solution of 72 (30 mg, 0.070 mmol) in DCM (2 mL) was treated with sodium triacetoxyborohydride (35 mg, 0.17 mmol), acetic acid (0.1 mL) and 3-Methoxy-4-hydroxybenzaldehyde (22 mg, 0.14 mmol) and the solution was allowed to stir under nitrogen for 1 h. The reaction was partitioned between ethyl acetate and and saturated NaHCO₃ solution, and the organic layer was washed two additional times with this solution. The organic layer was washed with brine, and it was dried with magnesium sulfate. The solvent was removed under reduced pressure. The crude was purified by flash chromatography in 5%MeOH/DCM to yield 73 (23 mg, 60%, 0.041 mmol) as a yellow oil. ¹H NMR (CDCl₃): δ 8.91 (d, J=4 Hz, 1H), 8.40 (d, J=8 Hz, 1H), 8.26 (d, J=8 Hz, 1H), 7.65-7.80 (m, 2H), 7.24 (m, 1H), 7.00 (s, 1H), 6.80 (m, 2H), 5.85 (dd, J=12 Hz, 4 Hz, 1H), 5.17 (bs, 1H), 3.96 (s, 3H), 3.61-3.87 (m, 12H), 3.30-3.58 (m, 5H), 2.61 (m, 1H), 1.26 (m, 2H). MS (ESI-POS):[M+H]+558; Anal. Calc. for C₃₁H₃₅N₅O₅: C, 66.77, H, 6.33, N, 12.56. Found: C, 63.53, H, 6.42, N, 9.17.

### EXAMPLE 30

The procedure of Example 10 was repeated, except that quinoline-3-carboxyaldehyde was substituted for 4-(dimethylamino)-1-naphthaldehyde in step (d). The remaining synthesis steps are detailed below

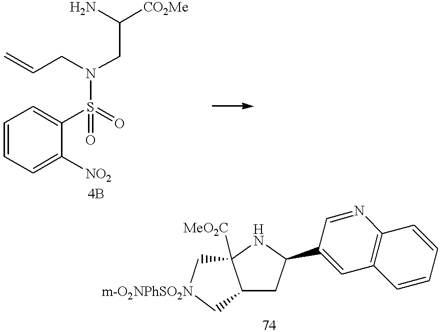

(a) Cyclization and Addition of the Naphthyl Moiety to Form (methyl (trans)-5-[(2-nitrophenyl)sulfonyl]-2-quinolin-3-ylhexahydropyrrolo[3,4-b]pyrrole-6a(1H)-carboxylate (74): A solution of 4B (500 mg, 1.5 mmol) in degassed toluene (150 mL) was treated with quinoline-3-carboxyaldehyde (366 mg, 2.3 mmol). It was allowed to stir 18 h at 90° C. The solvent was removed under reduced pressure. This crude material was chromatographed in 85% ethyl acetate/hexane, and the solvent was removed under reduced pressure to yield 74 (511 mg (71%), 1.1 mmol). ¹H NMR (CDCl₃): δ 8.95 (d, J=2 Hz, 1H), 8.05-8.16 (m, 3H), 8.10-8.23 (m, 3H), 7.60-7.88 (m, 5H), 7.48 (t, J=4 Hz, 1H), 4.78 (m, 1H), 3.92 (m, 2H), 3.79 (s, 3H), 3.72 (m, 2H), 3.27 (m, 1H), 2.21 (m, 1H), 2.10 (m, 1H). MS (ESI-POS):[M+H]+483.

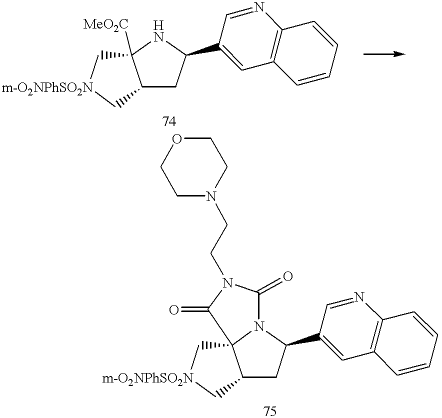

(b) Addition of the Morpholine Moiety to Provide (trans)-2-(2-morpholin-4-ylethyl)-8-[(2-nitrophenyl)sulfonyl]-5-quinolin-3-ylhexahydro-1H-pyrrolo[3′,4′:2,3]pyrrolo [1,2-c]imidazole-1,3(2H)-dione (75):

A solution of 74 (350 mg, 0.73 mmol) in DCM (12 mL) with DIEA (127 mg, 1.0 mmol) was cooled in an ice bath. Phosgene (0.58 mL of 2M solution, 1.2 mmol) was added, the solution turned dark green and it was allowed to stir for 1 h at room temperature. The DCM was removed under reduced pressure, and the crude material was partitioned between ethyl acetate and 1M Na₂CO₃ solution. The organic layer was washed twice with this sodium carbonate solution. It was then washed with brine, dried with MgSO₄., and the solvent was removed under reduced pressure. Ethyl acetate (20 mL), 4-(2-amino-1-ethyl)morpholine (511 mg, 3.7 mmol) and DIEA (470 mg, 3.7 mmol) were added, and it was allowed to stir for 4 h at 60° C. The ethyl acetate was washed with water twice, and once with brine. It was dried with MgSO₄, and the solvent was removed under reduced pressure to yield 75 (250 mg (23%), 0.4 mmol). ¹H NMR (CDCl₃): δ 8.40 (m , 1H), 7.92-8.10 (m, 4H), 7.60-7.82 (m, 5H), 5.52 (m, 1H), 3.61-3.87 (m, 12H), 3.30-3.58 (m, 5H), 2.61 (m, 1H), 1.26 (m, 2H). MS (ESI-POS):[M+H]+607.

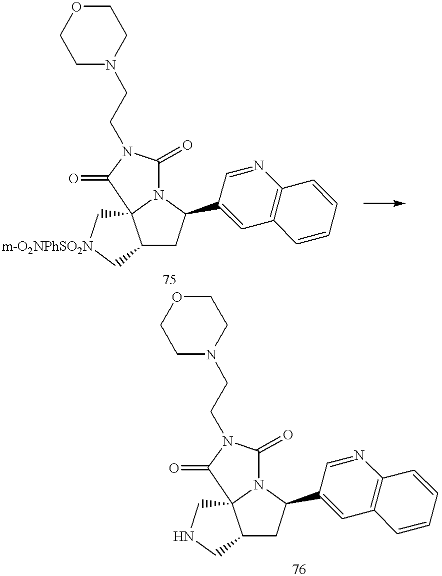

(c) Deprotection of the Amine Forming (trans)-2-(2-morpholin-4-ylethyl)-5-quinolin-3-ylhexahydro-1H-pyrrolo [3′,4′:2,3] pyrrolo [1,2-c]imidazole-1,3(2H)-dione (76):

A solution of 75 (150 mg, 0.24 mmol) in DMF (2 mL) was treated with sodium thiophenoxide (120 mg, 0.9 mmol) and the reaction was complete after 1 h. The reaction mixture was partitioned between ethyl acetate and 3N HCl. The organic layer was washed three times with 3N HCl. The organic layer was saved. The acidic water was combined and neutralized with sodium carbonate. The basic water was washed three times with ethyl acetate. All of the combined ethyl acetate was washed with brine, and dried with MgSO₄. The solvent was removed under reduced pressure to yield 76 (67 mg (68%), 0.16 mmol). ¹H NMR (CDCl₃): δ 8.92 (d, J=3 Hz, 1H), 8.26 (d, J=2 Hz, 1H), 8.07 (d, J=8 Hz, 1H), 7.95 (d, J=8 Hz, 1H), 7.75 (t, J=1 Hz, 1H), 7.61 (t, J=1 Hz, 1H), 5.30 (m, 1H), 3.61-3.87 (m, 12H), 3.30-3.58 (m, 5H), 2.63 (m, 1H), 1.27 (m, 2H). MS (ESI-POS): [M+H]+422.

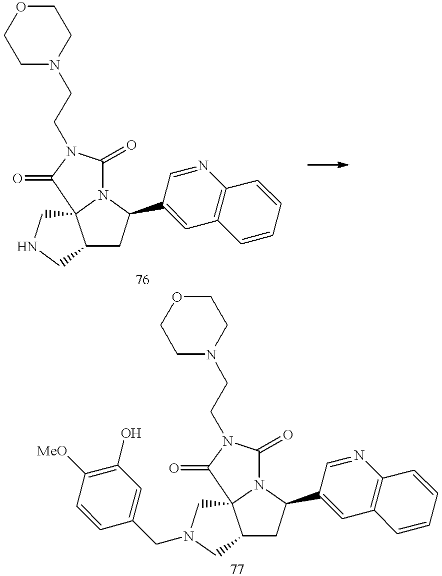

(d) Addition of the Benzyl Moiety to Provide the Final Product (trans)-8-(3-hydroxy-4-methoxybenzyl)-2-(2-morpholin-3-ylethyl)-5-quinolin-3-ylhexahydro-1H-pyrrolo [3′, 4′:2,3]pyrrolo[1,2-c]imidazole-1,3(2H)-dione (77): To a solution of 76 (40 mg, 0.1 mmol) and sodium triacetoxyborohydride (42 mg, 0.2 mmol) in DCM (2 mL) was added acetic acid (0.1 mL). 3-Methoxy-4-hydroxybenzaldehyde (30 mg, 0.2 mmol) was added and the solution was allowed to stir under nitrogen for 1 h. The reaction was partitioned between ethyl acetate and saturated NaHCO₃ solution, and the organic layer was washed two additional times with this solution. The organic layer was washed with brine, and it was dried with magnesium sulfate. The solvent was removed under reduced pressure. The crude was purified by flash chromatography in 5%MeOH/DCM to provide 77 (30 mg (54%), 0.054 mmol) as a yellow oil. ¹H NMR (CDCl₃): δ 8.91 (d, J=4 Hz, 1H), 8.26 (d, J=8 Hz, 1H), 8.06 (d, J=8 Hz, 1H), 7.65-7.80 (m, 2H), 7.24 (m, 1H), 7.00 (s, 1H), 6.80 (m, 2H), 5.85 (dd, J=12 Hz, 4 Hz, 1H), 5.17 (bs, 1H), 3.96 (s, 3H), 3.61-3.87 (m, 12H), 3.30-3.58 (m, 5H), 2.61 (m, 1H), 1.26 (m, 2H). MS (ESI-POS):[M+H]+558.

### EXAMPLE 31

### Biological Evaluation

(a) Preparation of COS-1 Cell Membranes Containing Human GnRH Receptors:

COS-1 cells infected with a recombinant adenovirus directing the expression of the human GnRH receptor were harvested 48 hours after virus infection using cell dissociation buffer from Gibco-BRL and pelleted by centrifugation (5 min., 1100 rpm in RC3B, 40 C). The pellet was suspended in 20 ml ice cold binding buffer (25 mM Tris HCl, pH 7.4, 0.1% sodium azide, 0.1% BSA) and homogenized using polytron (Tempest Virtishear, Virtis).

The homogenate was centrifuged for 12 min. at 14,500 rpm in a RC5B and the supernatant discarded. The pellet was re-homogenized in 20 ml of binding buffer and centrifuged. The final pellet was resuspended in a small volume of binding buffer such that the final protein concentration was approximately 1.5 mg/ml (according to Pierce-BCA Protein kit). Aliquots of membrane preparation could then be stored frozen at −70 C without significant loss of binding activity for future use.

(b) GnRH Radioligand Binding Assay:

Samples were diluted in binding buffer (25 mM Tris pH 7.5, 10 MM MgCl₂, 0.01% NaN₃, 0.1% BSA) for the assay. The radioligand used was ¹²⁵I-LHRH-D-TrP₆, approximately 50-70,000 counts/25 μl Non-specific binding was determined using LHRH-D-TrP₆ at a final concentration of 1 82 M. Addition of GnRH membranes to the assay was optimized to give a signal-to-noise ratio of 10:1.

Twenty-five μl sample (4× final concentration), 50 μl membranes and 25 μl radioligand were incubated in a V-bottom 96-well plate with shaking for 2 h at 4 C. The incubated mixture was then filtered onto a Whatman GF/B membrane pre-treated with 0.1% poly(ethylene imine) using a Packard Cell Harvester. The filter was dried for approximately 10 min. at 37 C. Forty μl Microscint 20 was added and the radioactivity remaining on the filter was counted using a Topcount γ-counter.

Binding inhibition was calculated from the measured values using a concentration series of each test compound in the conventional manner. Results are set forth in Table 2.

TABLE 2

| Compound #; Example # | Binding Assay IC₅₀ |
|---|---|
| AF21276; Example 1 | 35 ± 6 nM |
| AF20660; Example 2 | 800 ± 110 nM |
| AF21278; Example 3 | 280 ± 170 nM |
| AF21813; Example 4 | ˜80 nM |
| AF21477; Example 5 | ˜612 nM |
| AF21479; Example 6 | ˜160 nM |
| AF22352; Example 7 | ˜1500 nM |
| AF22053; Example 8 | ˜385 nM |
| 17; Example 13 | ˜321 nM |
| 36; Example 17 | ˜21 nM |
| 73; Example 29 | ˜1500 nM |
| 76; Example 30 | ˜270 nM |

1. A compound having the structural formula (I) 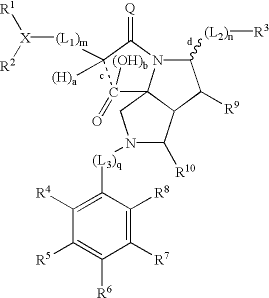
wherein:
L₁, L₂ and L₃ are independently linking groups;
m, n and q are independently 0 or 1;
c is an optional single bond, wherein, when c is present as a single bond, a and b are both 0, while when c is absent, a and b are both 1;
d represents a single bond that is either a or P;
Q is O or S;
X is N or CH;
R¹ and R² are either optionally substituted hydrocarbyl, in which case they may be the same or different, or R¹ and R² are linked together to form a five- or six-membered alicyclic or aromatic ring optionally containing 1 to 3 heteroatoms selected from the group consisting of N, O and S;
R³ is a cyclic structure containing 1 to 3 rings that may be fused or linked, wherein 1 or more of the rings may be aromatic and/or heterocyclic;
R⁴, R⁵, R⁶, R⁷ and R⁸ are independently selected from the group consisting of hydrogen, halogen, hydroxyl, alkyl, alkenyl, alkynyl, alkoxy, lower alkyl-substituted alkoxy, amino, lower alkyl-substituted amino, lower haloalkyl-substituted amino, amido, lower alkyl-substituted amido, lower haloalkyl-substituted amido, sulfonato, lower alkyl-substituted sulfonato, lower haloalkyl-substituted sufonato, nitro, nitrile and carboxyl, and, further, when two of R⁴, R⁵, R⁶, R⁷ and R⁸ are ortho to each other, they may together form a five- or six-membered cyclic structure containing 0 to 2 heteroatoms; and
R⁹ and R¹⁰ are independently selected from the group consisting of hydrogen, halogen, hydroxyl, alkyl, alkenyl, alkynyl, alkoxy, amino, lower alkyl-substituted amino, nitro, nitrile and carboxyl,
or a pharmaceutically acceptable salt thereof.

2. The compound of claim 1, wherein c represents a single bond and a and b are both 0.

3. The compound of claim 1, wherein c is absent, and a and b are both 1.

4. The compound of claim 1, wherein L₂ is lower alkylene and n is 1.

5. The compound of claim 1, wherein n is 0.

6. The compound of claim 1, wherein R³ is selected from the group consisting of phenyl and naphthalenyl, substituted with 0 to 2 substituents selected from the group consisting of hydroxyl, lower alkoxy, amino, and di(lower alkyl) amino.

7. The compound of claim 1, wherein m is 0.

8. The compound of claim 1, wherein L₁ is lower alkylene and m is 1.

9. The compound of claim 1, wherein q is 0.

10. The compound of claim 1, wherein q is 1.

11. The compound of claim 10, wherein X is N.

12. The compound of claim 1, wherein two of R⁴, R⁵, R⁶, R⁷ and R⁸ are hydrogen, and the remainder are independently selected from the group consisting of hydrogen, methoxy, carboxyl, acetyl, amido, phenyloxy, trifluoroamido, methylsulfamido, nitro and bromo.

13. The compound of claim 1, wherein R⁴, R⁵ and R⁸ are hydrogen, and R⁶ and R⁷ are linked together and represent —O—CH₂—CH₂—O—.

14. A compound having the structural formula (II) 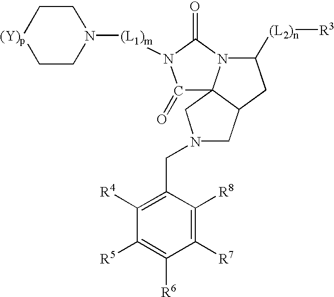
wherein:
L₁ and L₂ are independently lower alkylene linking groups;
m and n are independently 0 or 1;
R³ is a phenyl or naphthalenyl, substituted with a single lower alkoxy or di(lower alkyl)amino moiety;
Y is O, NH, S or CH₂, and p is 0 or 1; and
R⁴, R⁵, R⁶, R⁷ and R⁸ are independently selected from the group consisting of hydrogen, halogen, hydroxyl, alkyl, alkenyl, alkynyl, alkoxy, lower alkyl-substituted alkoxy, amino, lower alkyl-substituted amino, lower haloalkyl-substituted amino, amido, lower alkyl-substituted amido, lower haloalkyl-substituted amido, sulfonato, lower alkyl-substituted sulfonato, lower haloalkyl-substituted sufonato, nitro, nitrile and carboxyl, and, further, when two of R⁴, R⁵, R⁶, R⁷ and R⁸ are ortho to each other, they may together form a five- or six-membered cyclic structure containing 0 to 2 heteroatoms,
or a pharmaceutically acceptable salt thereof.

15. The compound of claim 14, wherein:
m is 0 or 1;
n is 0;
R³ is selected from the group consisting of phenyl and naphthalenyl, substituted with a single methoxy or dimethylamino group;
Y is O or CH₂, and p is 1;
R⁴ and R⁸ are hydrogen; and
either R⁵, R⁶ and R⁷ are hydrogen, methoxy, carboxyl, nitro or bromo, or R⁵ is hydrogen and R⁶ and R⁷ are linked together and represent —O—CH₂—CH₂—O—.

16. A GnRH receptor antagonistic composition comprising a therapeutically effective amount of the compound of claim 1 in combination with a pharmaceutically acceptable carrier.

17. The composition of claim 16, wherein the pharmaceutically acceptable carrier is suitable for oral administration and the composition comprises an oral dosage form.

18. A GnRH receptor antagonistic composition comprising a therapeutically effective amount of the compound of claim 14 in combination with a pharmaceutically acceptable carrier.

19. The composition of claim 18, wherein the pharmaceutically acceptable carrier is suitable for oral administration and the composition comprises an oral dosage form.

20. A method for antagonizing GnRH in a mammalian individual afflicted with a GnRH-related disorder, comprising administering to the individual a therapeutically effective amount of the compound of claim 1.

21. The method of claim 20, wherein the GnRH-related disorder is a sex hormone related condition.

22. The method of claim 21, wherein the sex hormone related condition is a sex hormone dependent cancer.

23. The method of claim 22, wherein the sex hormone dependent cancer is prostate cancer, uterine cancer, breast cancer, or pituitary gonadotrophe adenomas.

24. The method of claim 22, wherein the sex hormone dependent cancer is breast cancer.

25. The method of claim 21, wherein the sex hormone related condition is selected from the group consisting of endometriosis, polycystic ovarian disease, uterine fibroids and precocious puberty.

26. A method for preventing pregnancy in a fertile female subject, comprising administering a fertility-controlling amount of the compound of claim 1 to said subject.

27. A method for antagonizing GnRH in a mammalian individual afflicted with a GnRH-related disorder, comprising administering to the individual a therapeutically effective amount of the compound of claim 14.

28. The method of claim 27, wherein the GnRH-related disorder is a sex hormone related condition.

29. The method of claim 28, wherein the sex hormone related condition is a sex hormone dependent cancer.

30. The method of claim 29, wherein the sex hormone dependent cancer is prostate cancer, uterine cancer, breast cancer, or pituitary gonadotrophe adenomas.

31. The method of claim 30, wherein the sex hormone dependent cancer is breast cancer.

32. The method of claim 28, wherein the sex hormone related condition is selected from the group consisting of endometriosis, polycystic ovarian disease, uterine fibroids and precocious puberty.

33. A method for preventing pregnancy in a fertile female subject, comprising administering a fertility-controlling amount of the compound of claim 14 to said subject.

34. A method for synthesizing a bicyclic or tricyclic pyrrolidine derivative useful as a GnRH antagonist, comprising:
(a) providing a support bound molecule having the structural formula (IV) [chemical structure]
wherein S represents a solid support, Pr₁ and Pr₃ represent orthogonally removable protecting groups, L is a cleavable linker, and R⁹ and R¹⁰ are independently selected from the group consisting of hydrogen, halogen, hydroxyl, alkyl, alkenyl, alkynyl, alkoxy, amino, lower alkyl-substituted amino, nitro, nitrile and carboxyl;
(b) treating the support bound compound (IV) with a reagent effective to remove the protecting group Pr₁, followed by reaction with an aldehyde R³—(L₂)ₙ—CHO under conditions effective to form the imine (V) [chemical structure]
wherein n is 0 or 1, L₂ is a linking group, and R³ is a cyclic structure containing 1 to 3 rings that may be fused or linked, wherein 1 or more of the rings may be aromatic and/or heterocyclic;
(c) treating the imine (V) with reagents effective to bring about cyclization, thereby providing the bicyclic pyrrolidine derivative (VI) [chemical structure]
(d) contacting compound (VI) with phosgene or thiophosgene, followed by reaction with an amine derivative H₂N—(L₁)ₘ—X(R¹R²), to produce support-bound urea or thiourea analog (VII) [chemical structure]
wherein Q is O or S, L₁ is a linking group, m is 0 or 1, X is N or CH, and R¹ and R² are either optionally substituted hydrocarbyl, in which case they may be the same or different, or are linked together to form a five- or six-membered alicyclic or aromatic ring optionally containing 1 to 3 heteroatoms selected from the group consisting of N, O and S; and
(e) treating support bound urea or thiourea analog (VII) with a reagent effective to remove the protecting group Pr₃, followed by a reductive alkylation reaction with an aromatic reactant having the structural formula 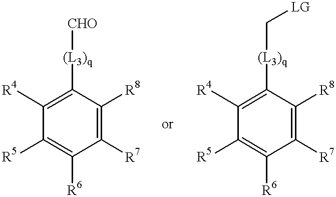
wherein L₃ is a linking group, q is 0 or 1, R⁴ through R¹ are independently selected from the group consisting of hydrogen, halogen, hydroxyl, alkyl, alkenyl, alkynyl, alkoxy, amino, lower alkyl-substituted amino, nitro, nitrile and carboxyl, or when two of R⁴ through R⁸ are ortho to each other, they may together form a five- or six-membered cyclic structure containing 0 to 2 heteroatoms, and LG is a leaving group, whereby the support-bound GnRH antagonist (VIII) [chemical structure]
is provided.

35. The method of claim 34, further including (f) releasing compound (VIII) from the solid support.

36. The method of claim 34, further including treating compound (VIII) with a reagent effective to bring about further cyclization and provide GnRH antagonist (IX) while releasing compound (IX) from the solid support.

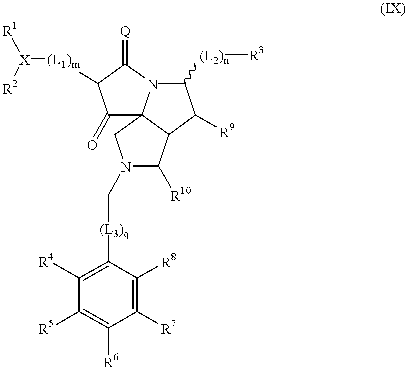

37. A method for synthesizing a bicyclic or tricyclic pyrrolidine derivative useful as a GnRH antagonist, comprising:
(a) providing a compound having the structural formula (XIII) 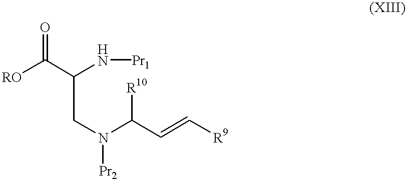
wherein R is a lower alkyl group, Pr₁ and Pr₂ represent orthogonally removable protecting groups, L is a cleavable linker, and R⁹ and R¹⁰ are independently selected from the group consisting of hydrogen, halogen, hydroxyl, alkyl, alkenyl, alkynyl, alkoxy, amino, lower alkyl-substituted amino, nitro, nitrile and carboxyl;
(b) treating the compound (XIII) with a reagent effective to remove the protecting group Pr₁, followed by reaction with an aldehyde R³—(L₂)ₙ—CHO under conditions effective to form the imine (XIV) 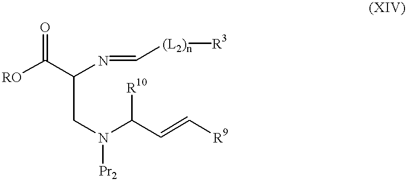
wherein n is 0 or 1, L₂ is a linking group, and R³ is a cyclic structure containing 1 to 3 rings that may be fused or linked, wherein 1 or more of the rings may be aromatic and/or heterocyclic;
(c) treating the imine (XIV) with reagents effective to bring about cyclization, thereby providing the bicyclic pyrrolidine derivative (XV) 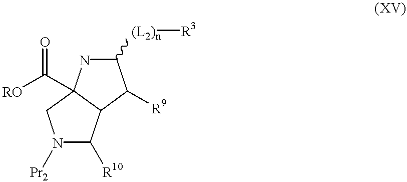
(d) contacting compound (XV) with phosgene or thiophosgene, followed by reaction with an amine derivative H₂N—(L₁)ₘ—X(R¹R²), to produce urea or thiourea analog (XVI) 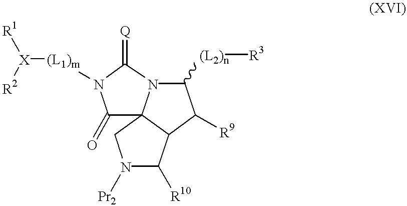
wherein Q is O or S, L₁ is a linking group, m is 0 or 1, X is N or CH, and R¹ and R² are either optionally substituted hydrocarbyl, in which case they may be the same or different, or are linked together to form a five- or six-membered alicyclic or aromatic ring optionally containing 1 to 3 heteroatoms selected from the group consisting of N, O and S; and
(e) treating urea or thiourea analog (XVI) with a reagent effective to remove the protecting group Pr₂, followed by a reductive alkylation reaction with an aromatic reactant having the structural formula 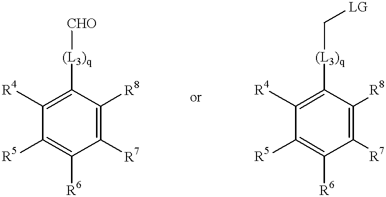
wherein L₃ is a linking group, q is 0 or 1, R⁴ through R⁸ are independently selected from the group consisting of hydrogen, halogen, hydroxyl, alkyl, alkenyl, alkynyl, alkoxy, lower alkyl-substituted alkoxy, amino, lower alkyl-substituted amino, lower haloalkyl-substituted amino, amido, lower alkyl-substituted amido, lower haloalkyl-substituted amido, sulfonato, lower alkyl-substituted sulfonato, lower haloalkyl-substituted sufonato, nitro, nitrile and carboxyl, or when two of R⁴ through R⁸ are ortho to each other, they may together form a five- or six-membered cyclic structure containing 0 to 2 heteroatoms, and LG is a leaving group, whereby the GnRH antagonist (XVII) 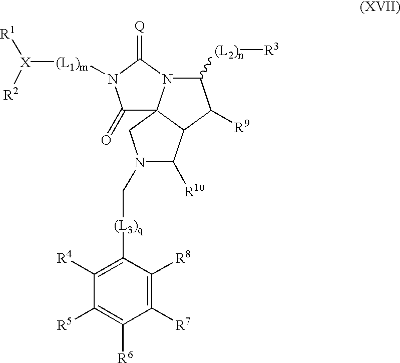
is provided.

38. The method of claim 34, further including, after step (e), (f) modifying R³.

39. The method of claim 34, further including, after step (e), (f) modifying any of R⁴, R⁵, R⁶, R⁷, and R⁸.

40. A method for antagonizing GnRH in a mammalian individual afflicted with a GnRH-related disorder, comprising administering to the individual a therapeutically effective amount of a tricyclic pyrrolidine derivative.

41. The method of claim 40, wherein the tricyclic pyrrolidine derivative contains the molecular fragment 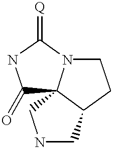
wherein is Q is O or S.

42. The method of claim 41, wherein the tricyclic pyrrolidine derivative has the structural formula (X) 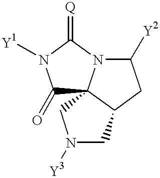
wherein Y¹, Y² and Y³ are independently optionally substituted hydrocarbyl of 1 to 24 carbon atoms.
